(19) United States
(12) Patent Application Publication
(10) Pub. No.: US 2026/0356808 A1
(43) Pub. Date: Nov. 12, 2026

(54) IONIZABLE LIPID AND USE THEREOF

(71) Applicant: GREEN CROSS CORPORATION, Yongin-si, Gyeonggi-do (KR)

(72) Inventors: Hyun Jung YOO, Yongin-si (KR); Ji Yeon SON, Yongin-si (KR); Jae Young BAN, Yongin-si (KR); Sun Hee KANG, Yongin-si (KR); Jung Hyun KIM, Yongin-si (KR); Jee Won KIM, Yongin-si (KR); Eun Young PARK, Yongin-si (KR); Jae Uk JEONG, Yongin-si (KR)

(73) Assignee: GREEN CROSS CORPORATION, Yongin-si, Gyeonggi-do (KR)

(21) Appl. No.: 19/109,628

(22) PCT Filed: Dec. 14, 2023
(86) PCT No.: PCT/KR2023/020679

§ 371 Date: Mar. 7, 2025

(30) Foreign Application Priority Data
Dec. 23, 2022 (KR) 10-2022-0182772

(51) Int. Cl. C07D 295/13; A61K 9/51; A61K 31/7088; A61K 48/00; C12N 15/88

(52) U.S. Cl. CPC C07D 295/13; A61K 9/5123; A61K 31/7088; A61K 48/0033; C12N 15/88

(57) ABSTRACT

A novel ionizable lipid compound, lipid nanoparticles including the ionizable lipid compound, and uses thereof are disclosed. The novel ionizable lipid compound is represented by formula (I) or a salt thereof. Lipid nanoparticles including a novel ionizable lipid compound have excellent nucleic acid encapsulation efficiency and high cell delivery efficiency of nucleic acids.

(Terminal area 1)ⱼ-connection1-headₖ-connection 2-(Terminal area 2)ₘ Formula (I).

### TECHNICAL FIELD

The present invention relates to a novel ionizable lipid or a salt thereof, lipid nanoparticles including the ionizable lipid or salt thereof, and a composition including an ionic drug and the ionizable lipid or salt thereof or the lipid nanoparticles.

### BACKGROUND ART

In the pharmaceutical field, research has been actively conducted on various particulate drug carriers as structures in the form of emulsions, liposomes, microparticles, and nanoparticles to enhance drug delivery efficiency into a body.

Meanwhile, with the advancement of biotechnology, the development and commercialization of medicines using nucleic acids such as RNA as active ingredients are actively being achieved. However, nucleic acids are vulnerable to degradation by enzymes in a body, such as nucleases, and are difficult to be delivered to target cells due to their size and anionic characteristics. Accordingly, drug carriers to protect nucleic acids from decomposition in a body while facilitating their delivery to target cells are in great need, and research is actively ongoing for lipid nanoparticles (LNPs).

LNPs are generally composed of ionizable lipids, helper lipids such as phospholipids, cholesterol, and PEG-lipids as main components. The ionizable lipids are known to play a key role in determining nucleic acid delivery efficiency by electrostatically binding to nucleic acids, encapsulating nucleic acids, and enabling nucleic acids to escape from endosomes within cells. In addition, the cholesterol keeps LNP particles hard and stable, the PEG-lipids play a role in preventing aggregation among LNP particles and ensuring structural stability, and the helper lipids (e.g., phospholipid) play a role in maintaining the lipid double membrane structure of the LNP particles.

Meanwhile, mRNA-based COVID-19 virus vaccines (e.g., Spikevax™, Comirnaty™) have recently been developed and achieved commercial success. The mRNA is known to be easily degraded in blood due to attack by RNase, etc., and has the disadvantage of being difficult to be delivered into cells due to its large molecular weight and negative charge. However, with the success of COVID-19 virus vaccine development with LNP system to overcome the shortcomings above, the importance of LNP technology in the pharmaceutical field is emerging.

Furthermore, in addition to mRNA, therapeutic development using various nucleic acids (e.g., antisense oligonucleotides (ASOs), microRNA (miRNA), etc.) is being actively performed. To allow nucleic acids therapeutics for clinical use, their inherent challenges (e.g., instability in a body and low delivery efficiency to cells) must be resolved, and there is a need to develop novel ionizable lipids that exhibit excellent cell delivery efficiency and nucleic acid encapsulation efficiency while minimizing nucleic acid degradation.

The present inventors synthesized and prepared a novel ionizable lipid compound having a branched ester structure produced by adding an ester group to a terminal area of an ionizable lipid or introducing hydrocarbons of various lengths at various positions around an ester functional group in a terminal area of the ionizable lipid. Then, the novel ionizable lipids with excellent cell delivery efficiency of nucleic acids were discovered.

### RELATED ART DOCUMENT

(Patent Document 1) US published patent US 2020/0140378 A1

### DISCLOSURE OF INVENTION

### Technical Problem

An object of the present invention is to provide a novel ionizable lipid compound that has excellent cell delivery efficiency and nucleic acid encapsulation efficiency while minimizing nucleic acid degradation, and to provide lipid nanoparticles containing the same, and a composition containing the same for drug delivery.

### Solution to Problem

In an embodiment of the present invention, provided is a compound represented by formula (I) or a salt thereof.

In another embodiment of the present invention, provided are lipid nanoparticles including the compound or salt thereof, a helper lipid, cholesterol, and a PEG lipid.

In another embodiment of the present invention, provided is a composition including an ionic drug and (i) the compound or salt thereof, or a composition including an ionic drug and (ii) the lipid nanoparticles.

### Advantageous Effects of Invention

A novel ionizable lipid compound or a salt thereof of the present invention allows excellent cell delivery efficiency and in vivo stability of nucleic acid, thereby contributing to improving the in vivo stability and therapeutic efficacy of nucleic acid therapeutics.

### BRIEF DESCRIPTION OF DRAWINGS

is FIG. 1 is a view showing a Z-average diameter and PI in F1-type LNPs including any of ionizable lipid compounds 2-7, 9-13, 27-30, 32, 33, 36-38, 40-43 and 46 to 49 herein.

FIG. 2 is a view showing an encapsulation efficiency in F1-type LNPs including any of ionizable lipid compounds 2-7, 9-13, 27-30, 32, 33, 36-38, 40-43 and 46-49 herein.

FIG. 3 is a view showing a Z-average diameter and PI of F2-type LNPs including any of ionizable lipid compounds 1-7, 9, 10, 12-26, and 28-49 herein.

FIG. 4 is a view showing an encapsulation efficiency in F2-type LNP including any of the ionizable lipid compounds 1-7, 9, 10, 12-26, and 28-49 herein.

FIG. 5 is a view showing the luminescence signal in a liver when control group (MC3 LNP) and an experimental group (F1-type LNP including an ionizable lipid compound 3, 4, 6, 32 or 46 herein) were administered via tail vein using firefly luciferase mRNA.

FIG. 6 is a view showing the luminescence signal in a liver when control group (MC3 LNP) and an experimental group (F2-type LNP including an ionizable lipid compound 3, 4, 6, 19, 22, 25, 44 or 45 herein) were administered via tail vein using firefly luciferase mRNA.

FIG. 7 is a view showing the luminescence signal in a liver when control group (MC3 LNP) and an experimental group (F1-type LNP including an ionizable lipid compound 4, 6, 32 or 46) were administered via tail vein using firefly luciferase mRNA.

FIG. 8 is a view showing the luminescence signal in a liver when control group (MC3 LNP) and an experimental group (F2-type LNP including an ionizable lipid compound 6, 19, 21, 22, 25, 44, 45 or 46 herein) were administered via intramuscular injection using firefly luciferase mRNA.

### BEST MODE FOR CARRYING OUT THE INVENTION

In an aspect of the present invention, the present invention relates to a compound or a salt thereof represented by the formula (I):

(terminal area 1)ⱼ-connection 1-headₖ-connection 2-(terminal area 2)ₘ (I),
the terminal areas 1 and 2 are each independently represented by formula (II) or formula (III),

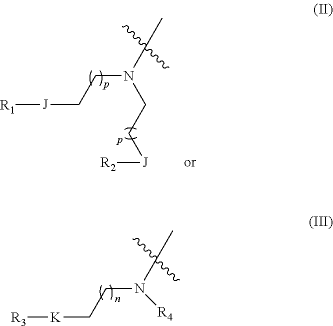

wherein J and K are each independently —C(═O)—O— or —O—C(═O)—,
R₁, R₂ and R₃ are each independently linear C₁₋₂₅ alkyl or branched C₃₋₃₅ alkyl, R₁ and R₂ are the same or different from each other, the linear C₁₋₂₅ alkyl or branched C₃₋₃₅ alkyl is substituted or unsubstituted with at least one selected from the group consisting of C₁₋₆ alkyl, Rx′-S—S—, Rx′-CH═CH—, Rx′-C(═O)—O—, Rx′-O—C(═O)—, —S—S—, —CH═CH—, —C(═O)—O—, —O—C(═O)—, 3- to 6-membered saturated or unsaturated carbocyclyl substituted or unsubstituted with Rx″, 3- to 6-membered saturated or unsaturated carbocycloalkyl substituted or unsubstituted with Rx″, 3- to 6-membered saturated or unsaturated heterocyclyl substituted or unsubstituted with Rx″, and 3- to 6-membered saturated or unsaturated heterocycloalkyl substituted or unsubstituted with Rx″,
R₄ is hydrogen, linear C₁₋₂₅ alkyl, branched C₃₋₃₀ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and is substituted or unsubstituted with one or more groups selected from the group consisting of Rx″, Rx′-CH═CH—, —CH═CH—, Rx′-C(═O)—O—, —C(═O)—O—, Rx′-O—C(═O)— and —O—C(═O)—,
Rx′ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and is substituted or unsubstituted with one or more Rx″,
Rx″ is a hydroxy group, linear C₁₋₁₅ alkyl, branched C₃₋₁₅ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl,
the wavy line indicates a binding position,
the terminal areas 1 and 2 are the same or different from each other,
the connections 1 and 2 are each independently a single bond, linear C₁₋₆ alkyl, branched C₃₋₆ alkyl or C₁₋₆ alkylene, which may or may not be substituted with a hydroxy group, —NR_{A}R_{B} or amine group, R_{A} and R_{B} are each independently linear C₁₋₆ alkyl, the connections 1 and 2 are the same or different from each other,
the head is a 6-membered heterocyclic amine or an aliphatic amine,
j and m are each independently an integer of 0 to 2, and cannot be an integer of 0 at the same time,
p and n are each independently integers from 1 to 20,
k is an integer of 0 or 1,
the heterocyclic amine is a cyclic amine group including one or more heteroatoms selected from N, S or O, and including at least one nitrogen atom,
the carbocyclyl is a cyclic group consisting of carbon and hydrogen atoms,
the heterocyclyl is a cyclic group including one or more heteroatoms selected from N, S or O,
the carbocycloalkyl is a carbocyclyl-linear C₁₋₆ alkyl group or a carbocyclyl-branched C₃₋₆ alkyl group, and
the heterocycloalkyl is a heterocyclyl-linear C₁₋₆ alkyl group or a heterocyclyl-branched C₃₋₆ alkyl group.

As used herein, the term “aliphatic amine” refers to a linear or branched hydrocarbon functional group having an amine terminus, which may have an aliphatic group having one or more unsaturated areas. The aliphatic amine is a functional group including at least one amine group and at least one aliphatic group, and includes, but not limited to, triethylamine, tripropylamine, tributylamine, diisopropylamine, triisopropylamine, triisobutylamine, N,N-diisopropylethylamine, stearylamine, ethylenediamine, propylenediamine, hexamethylenediamine, di(6-aminohexyl)amine, decylamine, undecylamine, dodecylamine, tridecylamine, tetradecylamine, hexadecylamine, octadecylamine, oleylamine, docosylamine, and the like.

As used herein, the term “substitution” refers to replacing one or more atoms (e.g., hydrogen atom, carbon atom) of an organic compound with another atomic group, and the substituent refers to the introduced atomic group. In addition, unless indicated to the contrary, individual substituents may optionally be further substituted (i.e., further substituted or unsubstituted). For example, terminal area 1 and terminal area 2 of formula (I) are represented by formula (II) in which R₁ and R₂ are linear alkyl, respectively, and a compound having the same structure as compound 34 of the present application is obtained when the linear alkyl is substituted with Rx′-CH═CH— and Rx′-C(═O)—O—. Furthermore, the term “substitution” includes the replacement of a hydrogen atom in a molecular structure with a substituent such that a chemically stable compound results from such substitution without exceeding the valence on the designated atom. For example, “group A is substituted with substituent B” or “group A has substituent B” may is mean that the hydrogen atom bonded to an atom such as carbon constituting the skeleton of group A is replaced by substituent B such that group A and substituent B form a covalent bond.

As used herein, the term “alkyl” refers to a functional group derived from a straight-chain or branched hydrocarbon, and includes, but not limited to, methyl, ethyl, N-propyl, i-propyl, N-butyl, i-butyl, t-butyl, N-pentyl, N-hexyl, and the like.

In addition, as used herein, the term “carbocyclyl” refers to a saturated carbocyclic group of carbon atoms having a single ring (e.g., cyclohexyl) as well as multiple fused rings (e.g., norbornyl, adamantyl), and a group (e.g. aryl, phenyl) having a partially unsaturated (e.g. cycloalkenyl) carbocyclic or aromatic ring, and includes, but not limited to, cyclopropyl, cyclopentyl, cyclohexyl, norbornyl, adamantyl, and the like. Moreover, as used herein, the term “heterocyclyl” refers to a mono- or poly-cyclic ring including at least one, preferably 1 to 4, heteroatoms O, N, or S, and does not include an aromatic ring. Examples include pyrrolidine, imidazoline, imidazolidine, pyrazoline, pyrazolidine, piperidine, morpholine, piperazine, tetrahydropyridinyl, and the like.

[1] In an embodiment of the present invention, the compound of the present invention relates to a compound of formula (I) or a salt thereof.

[2] In the embodiment of [1] above, k may be an integer of 1.

[3] In the present invention in the embodiment of [1] or [2] above, the head may be a 6-membered heterocyclic amine, or a 6-membered heterocyclic amine optionally including a nitrogen atom, or a 6-membered heterocyclic amine including at least 2 nitrogen atoms, or piperidine, pyridine, pyrimidine or piperazine.

[4] In the present invention in the embodiments of [1] to [3] above, the connection 1 may be linear C₁₋₆ alkyl, linear C₁₋₆ alkyl substituted with a hydroxy group, —NR_{A}R_{B}, or an amine group, branched C₃₋₆ alkyl, branched C₃₋₆ alkyl substituted with a hydroxy group, —NR_{A}R_{B}, or an amine group, C₁₋₆ alkylene, or C₁₋₆ alkylene substituted with hydroxy group, —NR_{A}R_{B}, or amine group, and R_{A} and R_{B} may each independently be linear C₁₋₆ alkyl or linear C₁₋₃ alkyl.

[5] In the present invention in the embodiments of [1] to [4] above, the connection 2 may be linear C₁₋₆ alkyl or linear alkyl substituted with a hydroxy group.

[6] In the present invention in the embodiments of [1] to [5] above, j and m may each be integers of 0 or 1 and at the same time not integers of 0.

[7] In the present invention in the embodiments of [1] to [6] above, R₁ is linear C₁₋₁₈ alkyl, linear C₂₋₁₈ alkyl, linear C₃₋₁₈ alkyl, linear C₄₋₁₈ alkyl, linear C₅₋₁₈ alkyl, linear C₆₋₁₈ alkyl, branched C₃₋₂₆ alkyl, branched C₄₋₂₆ alkyl, branched C₅₋₂₆ alkyl, branched C₆₋₂₆ alkyl, branched C₇₋₂₆ alkyl, or branched C₈₋₂₆ alkyl, and the linear alkyl or branched alkyl may be substituted or substituted with at least one selected from the group consisting of C₁₋₆ alkyl, Rx′-S—S—, Rx′-CH═CH—, Rx′-C(═O)—O—, Rx′-O—C(═O)—, —S—S—, —CH═CH—, —C(═O)—O—, —O—C(═O)—, 3- to 6-membered saturated or unsaturated carbocyclyl substituted or unsubstituted with Rx″, 3- to 6-membered saturated or unsaturated carbocycloalkyl substituted or unsubstituted with Rx″, 3- to 6-membered saturated or unsaturated heterocyclyl substituted or unsubstituted with Rx″, and 3- to 6-membered saturated or unsaturated heterocycloalkyl substituted or unsubstituted with Rx″.

[8] In the present invention in the embodiments of [1] to [7] above, R₁ may be substituted with at least one selected from the group consisting of linear C₂₋₁₈ alkyl, Rx′-C(═O)—O—, —CH═CH—, —C(═O)—O—, Rx′-O—C(═O)—, —O—C(═O)—, 3- to 6-membered saturated or unsaturated carbocyclyl substituted or unsubstituted with Rx″, and 3- to 6-membered saturated or unsaturated carbocycloalkyl substituted or unsubstituted with Rx″, and optionally may be substituted with at least one selected from the group consisting of linear C₂₋₁₈ alkyl, Rx′-C(═O)—O—, —CH═CH—, —C(═O)—O—, Rx′-O—C(═O)—, —O—C(═O)—, and 3- to 6-membered saturated or unsaturated carbocyclyl substituted or unsubstituted with Rx″, or may be substituted with at least one selected from the group consisting of linear C₂₋₁₈ alkyl, Rx′-C(═O)—O—, —C(═O)—O—, Rx′-O—C(═O)—, —O—C(═O)—, and 6-membered saturated or unsaturated carbocyclyl substituted or unsubstituted with Rx″.

[9] In the present invention in the embodiments of [1] to [8] above, R₂ is linear C₁₋₁₈ alkyl, linear C₂₋₁₈ alkyl, linear C₃₋₁₈ alkyl, linear C₄₋₁₈ alkyl, linear C₅₋₁₈ alkyl, linear C₆₋₁₈ alkyl, branched C₃₋₂₆ alkyl, branched C₄₋₂₆ alkyl, branched C₅₋₂₆ alkyl, branched C₆₋₂₆ alkyl, branched C₇₋₂₆ alkyl, or branched C₈₋₂₆ alkyl, and the linear alkyl or branched alkyl may be substituted or substituted with at least one selected from the group consisting of C₁₋₆ alkyl, Rx′-S—S—, Rx′-CH═CH—, —CH═CH—, Rx′-C(═O)—O—, —C(═O)—O—, Rx′-O—C(═O)—, —O—C(═O)—, 3- to 6-membered saturated or unsaturated carbocyclyl substituted or unsubstituted with Rx″, 3- to 6-membered saturated or unsaturated carbocycloalkyl substituted or unsubstituted with Rx″, 3- to 6-membered saturated or unsaturated heterocyclyl substituted or unsubstituted with Rx″, and 3- to 6-membered saturated or unsaturated heterocycloalkyl substituted or unsubstituted with Rx″.

[10] In the present invention in the embodiments of [1] to [9] above, R₂ may be substituted with at least one selected from the group consisting of linear C₂₋₁₈ alkyl, —CH═CH—, Rx′-C(═O)—O—, —C(═O)—O—, Rx′-O—C(═O)—, —O—C(═O)—, 3- to 6-membered saturated or unsaturated carbocyclyl substituted or unsubstituted with Rx″, and 3- to 6-membered saturated or unsaturated carbocycloalkyl substituted or unsubstituted with Rx″, and optionally may be substituted with at least one selected from the group consisting of linear C₂₋₁₈ alkyl, —CH═CH—, Rx′-C(═O)—O—, —C(═O)—O—, Rx′-O—C(═O)—, —O—C(═O)— and 3- to 6-membered saturated or unsaturated carbocyclyl substituted or unsubstituted with Rx″, or may be substituted with at least one selected from the group consisting of linear C₂₋₁₈ alkyl, —CH═CH—, Rx′-C(═O)—O—, —C(═O)—O—, Rx′-O—C(═O)—, —O—C(═O)—, and 6-membered saturated or unsaturated carbocyclyl substituted or unsubstituted with Rx″.

[11] In the present invention in the embodiments of [1] to [10] above, p and/or n may be each independently an integer of 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, or 1 to 6.

[12] In the present invention in the embodiments of [1] to [11] above, R₃ is linear C₁₋₁₈ alkyl, linear C₂₋₁₈ alkyl, linear C₃₋₁₈ alkyl, linear C₄₋₁₈ alkyl, linear C₆₋₁₈ alkyl, linear C₆₋₁₈ alkyl, branched C₃₋₃₂ alkyl, branched C₄₋₃₂ alkyl, branched C₅₋₃₂ alkyl, branched C₆₋₃₂ alkyl, branched C₇₋₃₂ alkyl, or branched C₈₋₃₂ alkyl, the linear alkyl or branched alkyl is linear C₁₋₁₄ alkyl, linear C₂₋₁₄ alkyl, linear C₃₋₁₄ alkyl, linear C₄₋₁₄ alkyl, linear C₅₋₁₄ alkyl, linear C₆₋₁₄ alkyl, or branched C₁₂₋₂₁ alkyl, and the linear alkyl or branched alkyl may be substituted or substituted with at least one selected from the group consisting of linear C₁₋₁₄ alkyl, linear C₂₋₁₄ alkyl, linear C₃₋₁₄ alkyl, linear C₄₋₁₄ alkyl, linear C₅₋₁₄ alkyl, linear C₆₋₁₄ alkyl, Rx′-CH═CH—, —CH═CH—, Rx′-C(═O)—O—, —C(═O)—O—, Rx′-O—C(═O)—, —O—C(═O)—, 3- to 6-membered saturated or unsaturated carbocyclyl substituted or unsubstituted with Rx″, 3- to 6-membered saturated or unsaturated carbocycloalkyl substituted or unsubstituted with Rx″, 3- to 6-membered saturated or unsaturated heterocyclyl substituted or unsubstituted with Rx″, and 3- to 6-membered saturated or unsaturated heterocycloalkyl substituted or unsubstituted with Rx″.

[13] In the present invention in the embodiments of [1] to [12] above, R₃ may be substituted or substituted with at least one selected from the group consisting of linear C₁₋₁₄ alkyl, linear C₂₋₁₄ alkyl, linear C₃₋₁₄ alkyl, linear C₄₋₁₄ alkyl, linear C₅₋₁₄ alkyl, linear C₆₋₁₄ alkyl, Rx′-CH═CH—, —CH═CH—, Rx′-C(═O)—O—, —C(═O)—O—, Rx′-O—C(═O)— and —O—C(═O)—.

[14] In the present invention in the embodiments of [1] to [13] above, R₄ may be hydrogen, linear C₁₋₂₅ alkyl, branched C₃₋₃₀ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and R₄ may be substituted or unsubstituted with one or more groups selected from the group consisting of Rx″, Rx′-CH═CH—, —CH═CH—, Rx′-C(═O)—O—, —C(═O)—O—, Rx′-O—C(═O)— and —O—C(═O)—, Rx′ may be linear C₁₋₂₅ alkyl, branched C₃₋₃₀ alkyl, linear C₅₋₃₀ alkenyl or branched C₅₋₃₀ alkenyl, and substituted or unsubstituted with one or more Rx″, and Rx″ may be a hydroxy group, linear C₁₋₁₅ alkyl, branched C₃₋₁₅ alkyl, 3- to 6-membered saturated or unsaturated carbocyclyl, or 3- to 6-membered saturated or unsaturated carbocycloalkyl.

[15] In the present invention in the embodiments of [1] to [14] above, R₄ may be hydrogen, linear C₁₋₂₅ alkyl, linear C₁₋₂₅ alkyl substituted with a hydroxy group, linear C₁₋₂₅ alkyl substituted with 6-membered saturated or unsaturated carbocyclyl, linear C₁₋₂₅ alkyl substituted with linear C₁₋₂₅ alkyl substituted with 6-membered saturated or unsaturated carbocycloalkyl, linear C₁₋₂₅ alkyl substituted with 6-membered saturated or unsaturated heterocyclyl, or linear C₁₋₂₅ alkyl or branched C₃₋₃₀ alkyl substituted with 6-membered saturated or unsaturated heterocycloalkyl, the linear alkyl or branched alkyl may be substituted or unsubstituted with one or more groups selected from the group consisting of Rx′-CH═CH—, —CH═CH—, Rx′-C(═O)—O—, —C(═O)—O—, Rx′-O—C(═O)— and —O—C(═O)—, and Rx′ may be linear C₁₋₂₅ alkyl, branched C₃₋₃₀ alkyl, linear C₅₋₃₀ alkenyl or branched C₅₋₃₀ alkenyl, and substituted or unsubstituted with one or more Rx″.

[16] In the present invention in the embodiments of [1] to [15] above, X and X₁ may each independently be oxygen atom (O).

[17] In the present invention in the embodiments of [1] to [16] above, both terminal areas 1 and 2 may be represented by formula (II), or both terminal areas 1 and 2 may be represented by formula (III), or either of the terminal areas 1 and 2 is represented by formula (II) and the remaining terminal area is represented by formula (III), or j of terminal area 1 is 0 and the terminal area 2 may be formula (II) or formula (III).

[18] In the present invention in the embodiment of [1] above, the present invention may be a compound represented by formula (V) below or a salt thereof:

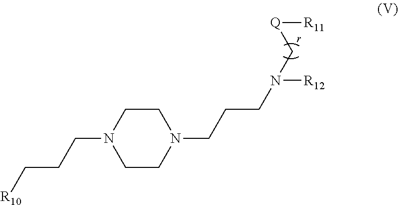

wherein r is an integer from 1 to 10,
Q is —C(═O)—O— or —O—C(═O)—
R₁₀ is linear C₁₋₂₅ alkyl, or branched C₃₋₃₅ alkyl, or is a group represented by formula (VI), the linear C₁₋₂₅ alkyl, or branched C₃₋₃₅ alkyl is substituted or unsubstituted with one or more groups selected from the group consisting of a hydroxy group, linear C₅₋₃₀ alkenyl, branched C₅₋₃₀ alkenyl, and —NR_{C}R_{D} and amine groups, R_{C} and R_{D} are each independently linear C₁₋₆ alkyl, and
R₁₂ is hydrogen, linear C₁₋₂₅ alkyl, linear C₁₋₂₅ alkyl substituted with a hydroxy group, linear C₁₋₂₅ alkyl substituted with 6-membered saturated or unsaturated carbocyclyl, 6-membered saturated or unsaturated carbocycloalkyl, or a group represented by formula (VI),

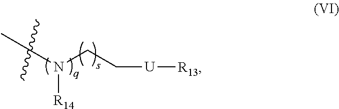

the wavy line indicates a binding position,
q is an integer of 0 or 1,
s is an integer from 1 to 10,
U is —C(═O)—O— or —O—C(═O)—
R₁₁, R₁₃ and R₁₄ are each independently hydrogen, linear C₁₋₂₅ alkyl, or branched C₃₋₃₅ alkyl or 6-membered saturated or unsaturated carbocycloalkyl, the linear C₁₋₂₅ alkyl, branched C₃₋₃₅ alkyl or 6-membered saturated or unsaturated carbocycloalkyl is substituted or unsubstituted with at least one selected from the group consisting of a hydroxy group, C₁₋₆ alkyl, linear C₅₋₃₀ alkenyl, branched C₅₋₃₀ alkenyl, Rₐ—S—S—, —S—S—, Rₐ—CH═CH—, —CH═CH—, Rₐ—C(═O)—O—, —C(═O)—O—, Rₐ—O—C(═O)—, —O—C(═O)—, 3- to 6-membered saturated or unsaturated carbocyclyl substituted or unsubstituted with R_{b}, 3- to 6-membered saturated or unsaturated carbocycloalkyl substituted or unsubstituted with
R_{b}, 3- to 6-membered saturated or unsaturated heterocyclyl substituted or unsubstituted with R_{b}, and 3- to 6-membered saturated or unsaturated heterocycloalkyl substituted or unsubstituted with R_{b},
Rₐ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and Rₐ is substituted or unsubstituted with R_{b},
R_{b} is linear C₁₋₁₅ alkyl, linear C₁₋₁₅ alkyl substituted with a hydroxy group, branched C₃₋₁₅ alkyl, 3- to 6-membered saturated or unsaturated carbocyclyl, 3- to 6-membered saturated or unsaturated carbocyclyl, 3- to 6-membered saturated or unsaturated cycloalkyl, 3- to 6-membered saturated or unsaturated heterocyclyl, or 3- to 6-membered saturated or unsaturated heterocycloalkyl,
the carbocyclyl is a cyclic group consisting of carbon and hydrogen atoms,
the heterocyclyl is a cyclic group including one or more heteroatoms selected from N, S or O,
the carbocycloalkyl is a carbocyclyl-linear C₁₋₆ alkyl group or a carbocyclyl-branched C₃₋₆ alkyl group,
the heterocycloalkyl is a heterocyclyl-linear C₁₋₆ alkyl group or a heterocyclyl-branched C₃₋₆ alkyl group, and preferably r and s may each independently be an integer of 1 to 9, 1 to 8, 1 to 7, 1 to 6, 1 to 5, or 1 to 4.

[19] In the present invention in the embodiment of [18] above, R₁₀ may be linear C₁₋₂₅ alkyl, branched C₃₋₃₅ alkyl, or a group represented by formula (VI), the linear C₁₋₂₅ alkyl, or branched C₃₋₃₅ alkyl may be substituted or unsubstituted with a hydroxy group, linear C₅₋₃₀ alkenyl, branched C₅₋₃₀ alkenyl, —NR_{C}R_{D} or an amine group, R_{C} and R_{D} may be each independently linear C₁₋₆ alkyl or linear C₁₋₃ alkyl, and
R₁₂ may be hydrogen, linear C₁₋₆ alkyl, linear C₁₋₂₅ alkyl substituted with a hydroxy group, linear C₁₋₆ alkyl substituted with 6-membered saturated or unsaturated carbocyclyl, 6-membered saturated or unsaturated carbocycloalkyl, or a group represented by formula (VI).

In another aspect of the present invention, the present invention may be a compound or a salt thereof, wherein k is an integer of 1 and the 6-membered heterocyclic amine of the head is piperidine, pyridine, pyrimidine, or piperazine.

In another aspect of the present invention, the present invention may be a compound or a salt thereof, wherein k is an integer of 1 and the head is an aliphatic amine.

In an aspect of the present invention, the present invention may be a compound or a salt thereof, wherein k is an integer of 1, the head is piperazine or piperidine, and the connections 1 and 2 are each independently linear C₁₋₆ alkyl substituted or unsubstituted with a hydroxy group, —NR_{A}R_{B} or an amine group, branched C₃₋₆ alkyl substituted or unsubstituted with a hydroxy group, —NR_{A}R_{B}, or an amine group, or C₁₋₆ alkylene substituted or unsubstituted with a hydroxy group, —NR_{A}R_{B} or an amine group.

In another aspect of the present invention, the present invention may be a compound in which both terminal areas 1 and 2 are represented by formula (II), or a salt thereof, a compound or a salt thereof in which the terminal areas 1 and 2 are both represented by formula (III), or a compound or salt thereof in which any one of the terminal areas 1 and 2 is represented by formula (II) and the remaining terminal area is represented by formula (III).

In an aspect of the present invention, the present invention may be a compound or a salt thereof, wherein p and/or n are integers of 1 to 10.

In another aspect of the present invention, the present invention may be a compound or a salt thereof, wherein R₁, R₂ and R₃ are each independently linear C₁₋₂₅ alkyl or branched C₃₋₃₂ alkyl substituted or unsubstituted with one or more selected from the group consisting of linear C₁₋₁₈ alkyl, 6-membered saturated or unsaturated carbocyclyl, 6-membered saturated or unsaturated carbocyclylalkyl, Rx′-CH═CH—, —CH═CH—, Rx′-C(═O)—O—, —C(═O)—O—, Rx′-O—C(═O)— and —O—C(═O)—, Rx′ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl and is substituted or unsubstituted with one or more Rx″, and Rx″ is a hydroxy group, linear C₁₋₁₅ alkyl, branched C₃₋₁₅ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl.

In another aspect of the present invention, the present invention may be a compound or a salt thereof, wherein R₁, R₂ and R₃ are each independently linear C₁₋₂₅ alkyl or branched C₃₋₃₂ alkyl substituted or unsubstituted with one or more selected from the group consisting of linear C₂₋₁₈ alkyl, Rx′-CH═CH—, —CH═CH—, Rx′-C(═O)—O—, —C(═O)—O—, Rx′-O—C(═O)— and —O—C(═O)—, 6-membered saturated or unsaturated carbocyclyl, 6-membered saturated or unsaturated carbocyclylalkyl, Rx′ is hydrogen, linear C₁₋₂₅ alkyl, branched C₃₋₂₅ alkyl, linear C₅₋₃₀ alkenyl, branched C₅₋₃₀ alkenyl, 6-membered saturated or unsaturated carbocyclyl, 6-membered saturated or unsaturated carbocyclyl, 6-membered saturated or unsaturated carbocycloalkyl, 6-membered saturated or unsaturated heterocyclyl, or 6-membered saturated or unsaturated heterocycloalkyl and Rx′ is substituted or unsubstituted with one or more Rx″, and Rx″ is a hydroxy group, linear C₁₋₁₅ alkyl, 6-membered saturated or unsaturated carbocyclyl, 6-membered saturated or unsaturated carbocyclylalkyl, 6-membered saturated or unsaturated heterocyclyl, or 6-membered saturated or unsaturated heterocyclylalkyl.

In another aspect of the present invention, the present invention may be a compound or a salt thereof, wherein one of the connections 1 and 2 is linear C₁₋₆ alkyl substituted or unsubstituted with a hydroxy group, —NR_{A}R_{B}- or an amine group, branched C₃₋₆ alkyl substituted with a hydroxy group, NR_{A}R_{B}— or amine group, or linear C₁₋₆ alkylene substituted or unsubstituted with a hydroxyl group, NR_{A}R_{B}— or an amine group, and R_{A} and R_{B} are each independently linear C₁₋₆ alkyl or linear C₁₋₃ alkyl.

In one aspect of the present invention, the present invention may be a compound or a salt thereof, wherein either of the terminal areas 1 and 2 is represented by formula (II) or j or m is 0, and the remaining terminal area is represented by formula (III). At this time, R₄ in formula (III) is hydrogen, linear C₁₋₂₅ alkyl, branched C₃₋₃₀ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and R₄ is substituted or unsubstituted with one or more groups selected from the group consisting of Rx″, Rx′-CH═CH—, —CH═CH—, Rx′-C(═O)—O—, —C(═O)—O—, Rx′-O—C(═O)— and —O—C(═O)—, Rx′ is linear C₁₋₂₅ alkyl or branched C₃₋₃₀ alkyl, and Rx″ is a hydroxy group, linear C₁₋₁₅ alkyl, branched C₃₋₁₅ alkyl, 3- to 6-membered saturated or unsaturated carbocyclyl, or 3- to 6-membered saturated or unsaturated carbocycloalkyl.

In another aspect of the present invention, the present invention may be a compound or a salt thereof, represented by formula (IV) below:

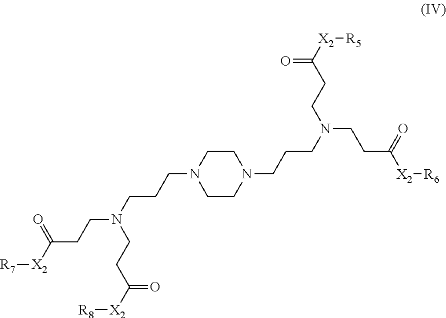

wherein the definition of X₂ is the same as the definition of X described above,
the definitions of R₅ to R₈ are the same as those of R₁ or R₂ described above, and R₅ to R₈ are the same or different from each other.

In another aspect of the invention, in the compound of formula (IV) or a salt thereof, X₂ is each independently O or NH, R₅ to R₈ are each independently linear C₃₋₁₅ alkyl or branched C₃₋₁₅ alkyl substituted or unsubstituted with at least one selected from the group consisting of Rx′-C(═O)—O—, Rx′-O—C(═O)—, Rx′-S—S— and Rx′-CH═CH—, Rx′ is linear C₁₋₁₀ alkyl substituted or unsubstituted with Rx″, and Rx″ is a hydroxy group, linear C₁₋₆ alkyl, 6-membered saturated or unsaturated carbocyclyl, or 6-membered saturated or unsaturated carbocyclylalkyl.

In an aspect of the present invention, the present invention may be a compound or a salt thereof, represented by formula (V) below:

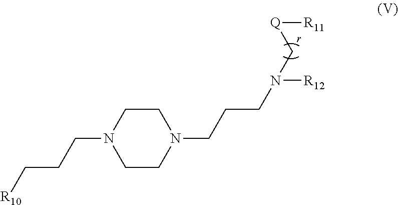

wherein r is an integer from 0 to 10, Q is —C(═O)—O— or —O—C(═O)—, R₁₀ is linear C₁₋₂₅ alkyl, or branched C₃₋₃₅ alkyl, or is a group represented by formula (VI), the linear C₁₋₂₅ alkyl, or branched C₃₋₃₅ alkyl is substituted or unsubstituted with a hydroxy group, —NR_{C}R_{D}, linear C₅₋₃₀ alkenyl, branched C₅₋₃₀ alkenyl, or amine groups, R_{C} and R_{D} are each independently linear C₁₋₆ alkyl, R₁₂ is hydrogen, linear C₁₋₂₅ alkyl, linear C₁₋₂₅ alkyl substituted with a hydroxy group, linear C₁₋₂₅ alkyl substituted with 6-membered saturated or unsaturated carbocyclyl, 6-membered saturated or unsaturated carbocycloalkyl, or a group represented by formula (VI),

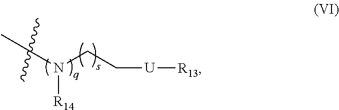

wherein the wavy line indicates a binding position, U is —C(═O)—O— or —O—C(═O)—, q is an integer of 0 or 1, s is an integer from 1 to 10, R₁₃ and R₁₄, are each independently hydrogen, linear C₁₋₂₅ alkyl, branched C₃₋₃₅ alkyl or 6-membered saturated or unsaturated carbocycloalkyl, the linear C₁₋₂₅ alkyl, branched C₃₋₃₅ alkyl or 6-membered saturated or unsaturated carbocycloalkyl is substituted or unsubstituted with at least one selected from the group consisting of a hydroxy group, C₁₋₆ alkyl, linear C₅₋₃₀ alkenyl, branched C₅₋₃₀ alkenyl, Rₐ—S—S—, —S—S—, Rₐ—CH═CH—, —CH═CH—, Rₐ—C(═O)—O—, —C(═O)—O—, Rₐ—O—C(═O)—, —O—C(═O)—, 3- to 6-membered saturated or unsaturated carbocyclyl substituted or unsubstituted with R_{b}, 3- to 6-membered saturated or unsaturated carbocycloalkyl substituted or unsubstituted with R_{b}, 3- to 6-membered saturated or unsaturated heterocyclyl substituted or unsubstituted with R_{b}, and 3- to 6-membered saturated or unsaturated heterocycloalkyl substituted or unsubstituted with R_{b}, Rₐ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and Rₐ is substituted or unsubstituted with R_{b}, R_{b} is linear C₁₋₁₅ alkyl, linear C₁₋₁₅ alkyl substituted with a hydroxy group, branched C₃₋₁₅ alkyl, 3- to 6-membered saturated or unsaturated carbocyclyl, 3- to 6-membered saturated or unsaturated carbocyclyl. cycloalkyl, 3- to 6-membered saturated or unsaturated heterocyclyl, or 3- to 6-membered saturated or unsaturated heterocycloalkyl, the carbocyclyl is a cyclic group consisting of carbon and hydrogen atoms, the heterocyclyl is a cyclic group including one or more heteroatoms selected from N, S or O, the carbocycloalkyl is a carbocyclyl-linear C₁₋₆ alkyl group or a carbocyclyl-branched C₃₋₆ alkyl group, and the heterocycloalkyl is a heterocyclyl-linear C₁₋₆ alkyl group or a heterocyclyl-branched C₃₋₆ alkyl group.

In an embodiment of the present invention, the present invention may be a compound or a salt thereof, wherein R₁₀ of formula (V) is linear C₁₋₂₅ alkyl, branched C₃₋₃₅ alkyl, or a group represented by formula (VI), the linear C₁₋₂₅ alkyl or branched C₃₋₃₅ alkyl is substituted or unsubstituted with a hydroxyl group, —NR_{C}R_{D}, linear C₅₋₃₀ alkenyl, branched C₅₋₃₀ alkenyl or an amine group, and R₁₂ is hydrogen, linear C₁₋₆ alkyl, linear C₁₋₂₅ alkyl substituted with a hydroxy group, linear C₁₋₆ alkyl substituted with 6-membered saturated or unsaturated carbocyclyl, 6-membered saturated or unsaturated carbocycloalkyl, or a group represented by formula (VI).

In another aspect of the present invention, the present invention may be a compound or a salt thereof, selected from the group consisting of:
(1) Tetrakis(2-ethylhexyl) 3,3′,3″,3′″-((piperazine-1,4-diylbis(propane-3,1-diyl))bis(azanetriyl)) tetrapropionate;
(2) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrapentanoate;
(3) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetraheptanoate;
(4) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetranonanoate;
(5) Tetra(octan-3-yl) 3,3′,3″,3′″-((piperazine-1,4-diylbis(propane-3,1-diyl))bis(azanetriyl))tetrapropionate;
(6) Tetrakis(4-(2-cyclohexylacetoxy)butyl) 3,3′,3″,3′″-((piperazine-1,4-diylbis(propane-3,1-diyl))bis(azanetriyl))tetrapropionate;
(7) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetraundecanoate;
(8) Tetrakis(4-(propionyloxy)butyl) 3,3′,3″,3′″-((piperazine-1,4-diylbis(propane-3,1-diyl))bis(azanetriyl)) tetrapropionate;
(9) ((3,3′,3″,3′″-((Piperazine-1,4-diylbis(propane-3,1-diyl))bis(azanetriyl))tetrakis(propanoyl))tetrakis(oxy)) tetrakis(ethane-2,1-diyl) tetraheptanoate;
(10) ((3,3′,3″,3′″-((Piperazine-1,4-diylbis(propane-3,1-diyl))bis(azanetriyl))tetrakis(propanoyl))tetrakis(oxy)) tetrakis(propane-3,1-diyl) tetrahexanoate;
(11) ((3,3′,3″,3′″-((Piperazine-1,4-diylbis(propane-3,1-diyl))bis(azanetriyl))tetrakis(propanoyl))tetrakis(oxy)) tetrakis(pentane-5,1-diyl) tetrabutyrate;
(12) Tetrakis(2-ethylhexyl) 4,4′,4″,4′″-((3,3′,3″,3′″-((piperazine-1,4-diylbis(propane-3,1-diyl))bis(azanetriyl)) tetrakis(propanoyl))tetrakis(oxy))tetrabutyrate;
(13) Tetrakis(2-ethylhexyl) 6,6′,6″,6′″-((3,3′,3″,3′″-((piperazine-1,4-diylbis(propane-3,1-diyl))bis(azanetriyl)) tetrakis(propanoyl))tetrakis(oxy))tetrahexanoate;
(14) ((3,3′,3″,3′″-((Piperazine-1,4-diylbis(propane-3,1-diyl))bis(azanetriyl))tetrakis(propanoyl))tetrakis(oxy)) tetrakis(hexane-6,1-diyl) tetrakis(2-hexyldecanoate);
(15) ((3,3′,3″,3′″-((Piperazine-1,4-diylbis(propane-3,1-diyl))bis(azanetriyl))tetrakis(propanoyl))tetrakis(oxy)) tetrakis(octane-8,1-diyl) tetrakis(2-hexyldecanoate);
(16) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octakis(decanoate);
(17) Tetrakis(2-ethylhexyl) 8,8′,8″,8′″-((3,3′,3″,3′″-((piperazine-1,4-diylbis(propane-3,1-diyl))bis(azanetriyl)) tetrakis(propanoyl))tetrakis(oxy))tetraoctanoate;
(18) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrakis(2-hexyldecanoate);
(19) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octahexanoate;
(20) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octaoctanoate;
(21) Tetrakis(2-propylhexyl) 4,4′,4″,4′″-((3,3′,3″,3′″-((piperazine-1,4-diylbis(propane-3,1-diyl))bis (azanetriyl))tetrakis(propanoyl))tetrakis(oxy))tetrabutyrate;
(22) Tetrakis(2-ethylpentyl) 4,4′,4″,4′″-((3,3′,3″,3′″-((piperazine-1,4-diylbis(propane-3,1-diyl))bis(azanetriyl)) tetrakis(propanoyl))tetrakis(oxy))tetrabutyrate;
(23) Tetrakis(2-propylhexyl) 6,6′,6″,6′″-((3,3′,3″,3′″-((piperazine-1,4-diylbis(propane-3,1-diyl))bis (azanetriyl))tetrakis(propanoyl))tetrakis(oxy))tetrahexanoate;
(24) Tetrakis(2-propylhexyl) 8,8′,8″,8′″-((3,3′,3″,3′″-((piperazine-1,4-diylbis(propane-3,1-diyl))bis (azanetriyl))tetrakis(propanoyl))tetrakis(oxy))tetraoctanoate;
(25) Tetrakis(2-ethylpentyl) 6,6′,6″,6′″-((3,3′,3″,3′″-((piperazine-1,4-diylbis(propane-3,1-diyl))bis(azanetriyl)) tetrakis(propanoyl))tetrakis(oxy))tetrahexanoate;
(26) Tetrakis(2-ethylpentyl) 8,8′,8″,8′″-((3,3′,3″,3′″-((piperazine-1,4-diylbis(propane-3,1-diyl))bis(azanetriyl)) tetrakis(propanoyl))tetrakis(oxy))tetraoctanoate;
(27) Di(henicosan-10-yl) 3,3′-((3-(4-(3-hydroxypropyl)piperazin-1-yl)propyl)azanediyl)dipropionate;
(28) ((3,3′-((3-(4-(2-Hydroxyethyl)piperazin-1-yl)propyl)azanediyl)bis(propanoyl))bis(oxy))bis(hexane-6,1-diyl) bis(2-hexyldecanoate);
(29) ((3,3′-((3-(4-(3-Hydroxypropyl)piperazin-1-yl)propyl)azanediyl)bis(propanoyl))bis(oxy))bis(hexane-6,1-diyl) bis(2-hexyldecanoate);
(30) Di(henicosan-10-yl) 3,3′-((piperazine-1,4-diylbis(propane-3,1-diyl))bis(azanediyl))dipropionate;
(31) Di(henicosan-10-yl) 3,3′-((piperazine-1,4-diylbis(propane-3,1-diyl))bis(benzylazanediyl))dipropionate;
(32) Di(henicosan-10-yl) 3,3′-((piperazine-1,4-diylbis(propane-3,1-diyl))bis(methylazanediyl))dipropionate;
(33) Tetra((Z)-non-2-en-1-yl) 9,9′,9″,9′″-((piperazine-1,4-diylbis(propane-3,1-diyl))bis(azanetriyl)) tetranonanoate;
(34) Tetra((Z)-non-2-en-1-yl) 6,6′,6″,6′″-((3,3′,3″,3′″-((piperazine-1,4-diylbis(propane-3,1-diyl))bis (azanetriyl))tetrakis(propanoyl))tetrakis(oxy))tetrahexanoate;
(35) Tetra((Z)-non-2-en-1-yl) 4,4′,4″,4′″-((3,3′,3″,3′″-((piperazine-1,4-diylbis(propane-3,1-diyl))bis (azanetriyl))tetrakis(propanoyl))tetrakis(oxy))tetrabutyrate;
(36) Di(henicosan-10-yl) 3,3′-((3-(4-(3-((3-(henicosan-10-yloxy)-3-oxopropyl)amino)propyl)piperazin-1-yl) propyl)azanediyl)dipropionate;
(37) (6Z,16Z)-12-((Z)-Dec-4-en-1-yl)docosa-6,16-dien-11-yl 3-((3-(4-(2-hydroxyethyl)piperazin-1-yl) propyl)(methyl)amino)propanoate;
(38) (6Z,16Z)-12-((Z)-Dec-4-en-1-yl)docosa-6,16-dien-11-yl 3-((3-(4-(3-(dimethylamino)propyl)piperazin-1-yl)propyl)(methyl)amino)propanoate;
(39) Bis((6Z,16Z)-12-((Z)-dec-4-en-1-yl)docosa-6,16-dien-11-yl) 3,3′-((piperazine-1,4-diylbis(propane-3,1-diyl))bis(methylazanediyl))dipropionate;
(40) Tetrakis(2-(2-cyclohexylacetoxy)ethyl) 3,3′,3″,3′″-((piperazine-1,4-diylbis(propane-3,1-diyl))bis (azanetriyl))tetrapropionate;
(41) Bis(4-(2-cyclohexylacetoxy)butyl) 3,3′-((piperazine-1,4-diylbis(propane-3,1-diyl))bis(methylazanediyl)) dipropionate;
(42) (((3,3′-((Piperazine-1,4-diylbis(propane-3,1-diyl))bis(methylazanediyl))bis(propanoyl))bis(oxy))bis(methylene))bis(propane-2,1,3-triyl) tetrahexanoate;
(43) Di(octan-3-yl) 3,3′-((piperazine-1,4-diylbis(propane-3,1-diyl))bis(methylazanediyl))dipropionate;
(44) (((3,3′-((Piperazine-1,4-diylbis(propane-3,1-diyl))bis((3-(octan-3-yloxy)-3-oxopropyl)azanediyl))bis(propanoyl))bis(oxy))bis(methylene))bis(propane-2,1,3-triyl) tetrahexanoate;
(45) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrakis(3-ethylpentanoate);
(46) Di(henicosan-10-yl) 3,3′-((piperazine-1,4-diylbis(propane-3,1-diyl))bis((4-hydroxybutyl)azanediyl)) dipropionate;
(47) Bis(2-ethylhexyl) 3,3′-((3-(4-(3-(dimethylamino)propyl)piperazin-1-yl)propyl)azanediyl)dipropionate;
(48) ((3-(4-(3-(Dimethylamino)propyl)piperazin-1-yl)propyl)azanediyl)bis(hexane-6,1-diyl) bis(2-hexyldecanoate); and
(49) Di((Z)-non-2-en-1-yl) 9,9′-((3-(4-(3-(dimethylamino)propyl)piperazin-1-yl)propyl)azanediyl) dinonanoate.

In an aspect of the present invention, the present invention may be a lipid nanoparticle including the compound or a salt thereof, helper lipid, cholesterol, and PEG lipid.

As used herein, the term “lipid nanoparticle (LNP)” refers to a structure that mixes lipids with drugs (e.g., nucleic acid-based drugs) at room temperature to prepare a uniform phase and disperses the same in an aqueous solution so that the drug exists as a solid solution between lipid crystals. Since using substances, such as phospholipids, lipids, and cholesterol, which exist in living organisms, the lipid nanoparticles are particulate drug carriers that have high bioavailability and affinity, enable drug release, and control, and have high stability against decomposition by enzymes or the like. In addition, lipid nanoparticles are positively (+) charged at acidic pH to encapsulate RNA, and are neutrally charged at physiological pH to minimize toxicity.

In another aspect of the invention, the helper lipid may be a phospholipid. The phospholipid is a lipid including a phosphate group and one or more fatty acid chains, which may include one or more multiple bonds and may interact with one or more negatively charged phospholipids of a membrane (e.g., a cell membrane).

In an embodiment of the present invention, the phospholipid may be used without is limitation as long as it is a phospholipid capable of promoting fusion of lipid nanoparticles. As the phospholipid, one or more phospholipids selected from the group consisting of DOPE(dioleoylphosphatidylethanolamine; 1,2-dioleoyl-sn-glycero-3-phosphoethanolamine), DSPC(distearoylphosphatidylcholine), POPC(palmitoyloleoylphosphatidylcholine; 1-palmitoyl-2-oleoyl-sn-glycero-3-phosphocholine), EPC(egg phosphatidylcholine), DOPC(dioleoylphosphatidylcholine), DPPC(dipalmitoylphosphatidylcholine), DOPG(dioleoylphosphatidylglycerol), DPPG(dipalmitoylphosphatidylglycerol), DSPE(distearoylphosphatidylethanolamine), PE(phosphatidylethanolamine), DPPE(dipalmitoylphosphatidylethanolamine), POPE(1-palmitoyl-2-oleoyl-sn-glycero-3-phosphoethanolamine), DOPS(1,2-dioleoyl-sn-glycero-3-[hosphor-L-serine]), 2-dioctadecanoyl-sn-glycero-3-phosphoserine (DSPS; 18:0 PS), 1,2-di-(9Z-octadecenoyl)-sn-glycero-3-phospho-L-serine (DOPS; 18:1 Ps), sphingomyelin, and the like may be used, and may be preferably one or more selected from DSPC, DOPC, DSPE, DOPE, or sphingomyelin, but is not limited thereto.

In another aspect of the invention, the PEG-lipid may include a linker moiety suitable for linking PEG to lipid nanoparticles. The linker moiety may be one or more selected from the group consisting of carbonate (—OC(O)O—), succinoyl, phosphate ester (—O—(O)POH—O—), sulfonate ester, amido (—C(O)NH—), amino (—NR—), carbonyl (—C(O)—), carbamate (—NHC(O)O—), urea (—NHC(O)NH—), disulfide (—SS—), ether (—O—), succinyl (—(O)CCH₂CH₂C(O)—), and succinamidyl (—NHC(O)CH₂CH₂C(O)NH—), but is not limited thereto.

In an aspect of the present invention, helper lipids and/or cholesterol of the lipid nanoparticles may be used as lipids constituting the PEG-lipid, but any lipid that can bind to PEG may be used without limitation.

In an aspect of the present invention, the molar ratio, weight ratio, or volume ratio of the combination of the helper lipid, cholesterol, and PEG lipid components of the lipid nanoparticles of the present invention and the compound or salt thereof may be 1:10 to 10:1, but is not limited thereto.

In another aspect of the invention, the lipid nanoparticles of the invention may include 5 to 20 mole % helper lipid, 20 to 60 mole % cholesterol, and 0.5 to 5 mole % PEG-lipid. At this time, mole % refers to the percentage obtained by dividing the number of moles of a specific component by the sum of the number of moles of all components.

In an aspect of the present invention, the present invention may be a composition including an ionic drug and (i) the compound or a salt thereof, or a composition including an ionic drug and (ii) the lipid nanoparticles.

In another aspect of the present invention, the ionic drug may be any one or more selected from the group consisting of nucleic acid or nucleic acid-based drugs, peptides, protein drugs, protein-nucleic acid constructs, and ionic biopolymer-drug conjugates.

In an embodiment of the present invention, the nucleic acid may be, but not limited to, one or more selected from the group consisting of antisense oligonucleotide (ASO), short interfering RNA (siRNA), micro RNA (miRNA), self amplifying RNA (SAM), circular RNA, messenger RNA (mRNA), crRNA, tracrRNA, single guide RNA (sgRNA), transfer RNA (tRNA), asymmetric interference RNA (aiRNA), antagomir, ribozyme, dicer substrate RNA, short hairpin RNA (shRNA), plasmid DNA (pDNA), double stranded DNA (dsDNA), partial double stranded DNA, triple stranded DNA, partial triple stranded DNA, single stranded DNA (ssDNA), double stranded RNA (dsRNA), locked nucleic acid (LNA), peptide nucleic acid (PNA), miRNA analog or anti-miRNA.

In another embodiment of the present invention, the ionic drug may be a therapeutic agent to treat or prevent a disease.

In another aspect of the present invention, the composition can be used as a pharmaceutical composition for treating diseases.

In another aspect of the present invention, a composition including an ionic drug and (i) the compound or salt thereof, or a composition including an ionic drug and (ii) the lipid nanoparticles may exhibit a biologically equivalent or increased drug delivery effect, when compared to compositions including an ionic drug together with previously known ionic lipids such as MC3.

In another aspect of the invention, an ionic drug and (i) a composition including the compound or salt thereof, or a composition including an ionic drug and (ii) the lipid nanoparticles may have a biologically equivalent or increased disease treatment or prevention effect or may have improved immunogenicity, when compared to is compositions including the same ionic drug together with previously known lipids such as MC3.

In an aspect of the present invention, the composition may include conventional non-toxic pharmaceutically acceptable additives. Additives that may be used in the composition of the present invention include sweeteners, binders, solubilizers, solubilizing agents, wetting agents, emulsifiers, isotonic agents, adsorbents, disintegrants, antioxidants, preservatives, lubricants, fillers, fragrances, and the like, for example, lactose, dextrose, sucrose, mannitol, sorbitol, cellulose, glycine, silica, talc, stearic acid, sterine, magnesium stearate, magnesium aluminum silicate, starch, gelatin, gum tragacanth, arginic acid, sodium alginate, methylcellulose, sodium carboxymethylcellulose, agar, water, ethanol, polyethylene glycol, polyvinylpyrrolidone, sodium chloride, calcium chloride, orange essence, strawberry essence, vanilla flavor, and the like.

The composition of the present invention may be prepared in various oral dosage forms such as tablets, pills, powders, capsules, syrups or emulsions, or in parenteral dosage forms such as intramuscular, intravenous or subcutaneous administration.

When the composition of the present invention is formulated in an oral dosage form, cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and the like may be used as additives.

In addition, when the composition of the present invention is formulated in an injection form, water, saline solution, glucose aqueous solution, similar sugar aqueous solution, alcohol, glycol, ether, oil, fatty acid, fatty acid ester, glyceride, surfactant, suspending agents, emulsifiers, and the like may be used as additives.

Hereinafter, to help understanding, the present invention will be described in detail through examples and the like. However, the embodiments according to the present invention may be modified into various other forms, and the scope of the present invention should not be construed as being limited to the following embodiments.

### Preparation Example 1. Synthesis of Ionizable Lipids

The synthetic formula below merely illustrates a method for producing the novel is ionizable lipid compound of the present invention and is not intended to limit the scope of the present invention as defined in the appended claims.

A novel ionizable lipid compound was synthesized based on the general [Synthetic Formula 1] to [Synthetic Formula 7] below.

### [Synthesis Formula 1] Overall Reaction Formula for Ionizable Lipid Compounds 1 to 26, 33 to 36, 40, 44 and 45

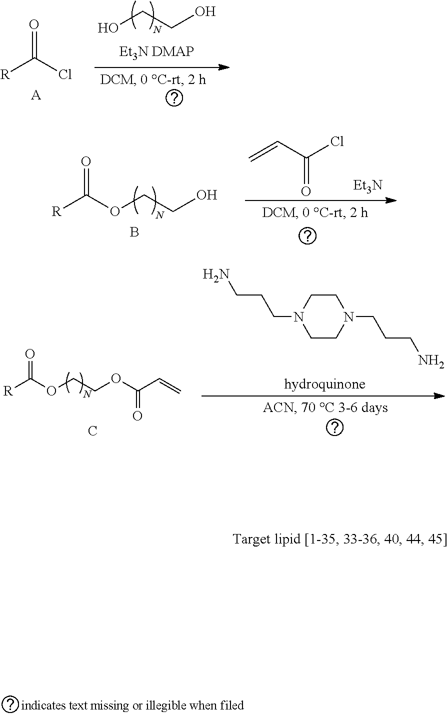

n is an integer of 1 or more
R: Linear alkyl, branched alkyl or oxycarbonyl alkyl

### Step 1: Synthesis of Compound B

To a stirred solution of diol (1.2 eq.) in dichloromethane (DCM) were added triethylamine (Et₃N, 1.0 eq.), 4-dimethylamoniopyridine (DMAP, 0.1 eq.) followed by an addition of alkyloyl chloride, compound A (1.0 eq.) drop wise at 0 °C and the reaction mixture was allowed to stir at room temperature for 2 h. The progress of the reaction was monitored by TLC. After completion of reaction, the reaction mixture was diluted with water and extracted with dichloromethane (twice). The combined organic layer was dried over anhydrous Na₂SO₄, filtered and the filtrate was concentrated under reduced pressure to give crude compound. The crude compound was purified by silica gel (100-200 mesh) column chromatography to afford compound B.

### Step 2: Synthesis of Compound C

To a stirred solution of compound B (1.0 eq.) in dichloromethane were added triethylamine (2.0 eq.), acryloyl chloride (0.38 mL, 4.65 mmol, 1.2 eq.) drop wise at 0 °C and the reaction mixture was allowed to stir at room temperature for 2 h. The progress of the reaction was monitored by TLC. After completion of reaction, the reaction mixture was diluted with water and extracted with dichloromethane. The combined organic layer was dried over anhydrous Na₂SO₄, filtered and the filtrate was concentrated under reduced pressure to give crude compound. The crude compound was purified by silica gel (100-200 mesh) column chromatography to afford compound C.

### Step 3: Synthesis of Ionizable Lipid Compounds 1 to 26, 33 to 36, 40, 44 and 45

To a stirred mixture of 3,3′-(piperazine-1,4-diyl)bis(propan-1-amine) (1.0 eq.), compound C (4.0 eq.) in acetonitrile (ACN) was added catalytic amount of hydroquinone (0.1 eq.) at room temperature. The reaction mixture was heated to 70 °C and stirred at the same temperature. The progress of the reaction was monitored by TLC. After completion of reaction, the reaction mixture was diluted with water and extracted with ethyl acetate. The combined organic layer was dried over anhydrous Na₂SO₄, filtered and the filtrate was concentrated under reduced pressure to give crude compound. The crude compound was purified by silica gel (100-200 mesh) column chromatography to afford ionizable lipid compounds, 1-26, 33-36, 40, 44, 45.

### [Synthesis Formula 2] Overall Reaction Formula for Ionizable Lipid Compounds 27 to 29

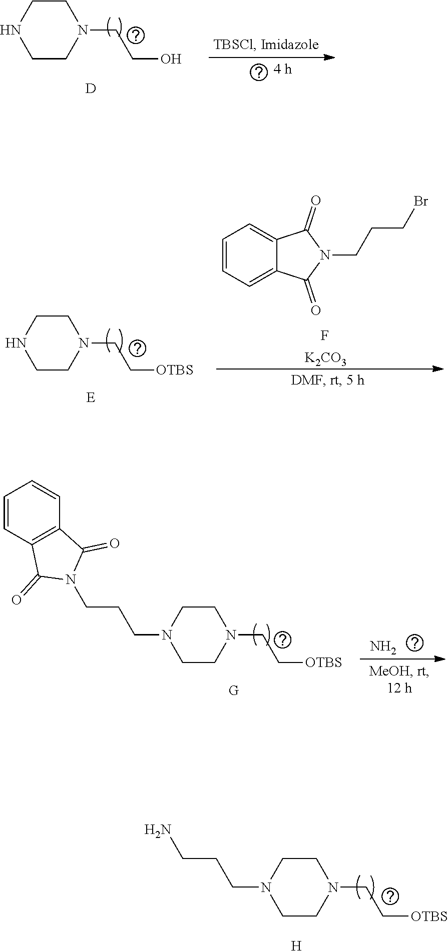

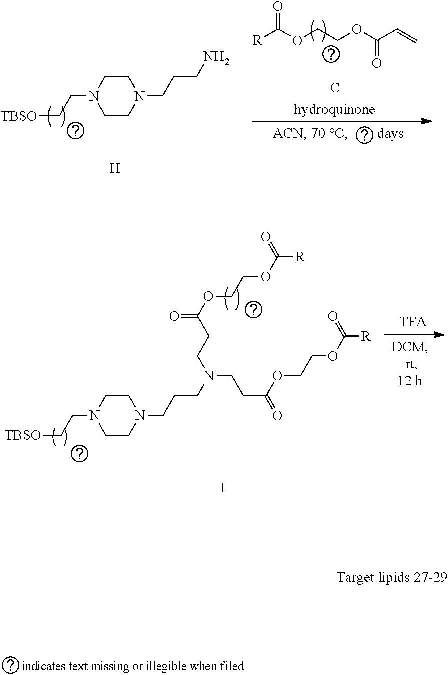

n is an integer of 1 or more
R: Linear alkyl, branched alkyl or oxycarbonyl alkyl

### Step 1: Synthesis of Compound E

To a stirred solution of compound D (1.0 eq.) in dichloromethane, imidazole (1.0 eq.) and TBDMS-Cl (TBSCI, tert-Butyldimethylsilyl chloride, 1.5 eq.) was added drop wise at 0 °C. The reaction mixture was allowed to stir at room temperature for 4 h. Progress of the reaction was monitored by TLC. After completion of reaction, the reaction mixture was diluted with water and extracted with dichloromethane (twice). The combined organic layer was dried over anhydrous Na₂SO₄, filtered and the filtrate was concentrated under reduced pressure to give crude compound. The crude compound was purified by silica gel (100-200 mesh) column chromatography, eluted with 10% methanol/DCM to afford compound E

### Step 2: Synthesis of Compound G

To a stirred solution of compound E (1.0 eq.) in DMF (N,N-Dimethylformamide), K₂CO₃ (3.0 eq.) and compound F (1.2 eq.) were added and stirred at room temperature for 5 h. Progress of the reaction was monitored by TLC. After completion of reaction, the reaction mixture was diluted with water and extracted with ethyl acetate (twice). The combined organic layer was dried over anhydrous Na₂SO₄, filtered and the filtrate was concentrated under reduced pressure to give crude compound. The crude compound was purified by silica gel (100-200 mesh) column chromatography, eluted with 3% ethyl acetate/petroleum ether to afford compound G.

### Step 3: Synthesis of Compound H

To a stirred solution of compound G (1.0 eq.) in methanol, hydrazine hydrate (10 eq.) was added and stirred at room temperature for 12 h. Progress of the reaction was monitored by TLC. After completion of reaction, the reaction mixture was concentrated under reduced pressure to give crude compound. The crude compound was purified by silica gel (100-200 mesh) column chromatography, eluted with 4% ethyl acetate/petroleum ether to afford compound H.

### Step 4: Synthesis of Compound I

To a stirred mixture of compound C (2.5 eq.), compound H (1.0 eq.) in acetonitrile was added catalytic amount of hydroquinone (0.1 eq.) at room temperature. The reaction mixture was heated to 70 °C and stirred at the same temperature for 48 h. Progress of the reaction was monitored by TLC. After completion of reaction, the reaction mixture was concentrated under reduced pressure to afford crude compound I.

### Step 5: Synthesis of Ionizable Lipid Compounds 27 to 29

To a stirred solution of compound I (1.00 eq.) in DCM was added TFA (Trifluoroacetic acid, 5 eq.) at room temperature. The reaction mixture was then stirred at the same temperature for 12 h. Progress of the reaction was monitored by TLC. After completion of reaction, the reaction mixture was quenched with saturated NaHCO₃ solution and extracted with DCM (twice). The combined organic layer was dried over anhydrous Na₂SO₄, filtered and the filtrate was concentrated under reduced pressure to give crude compound. The crude compound was purified by silica gel (100-200 mesh) column chromatography, eluted with 6% methanol/dichloromethane to afford ionizable lipid compounds 27-29.

### [Synthesis Formula 3] Overall Reaction Formula for Ionizable Lipid Compound 37

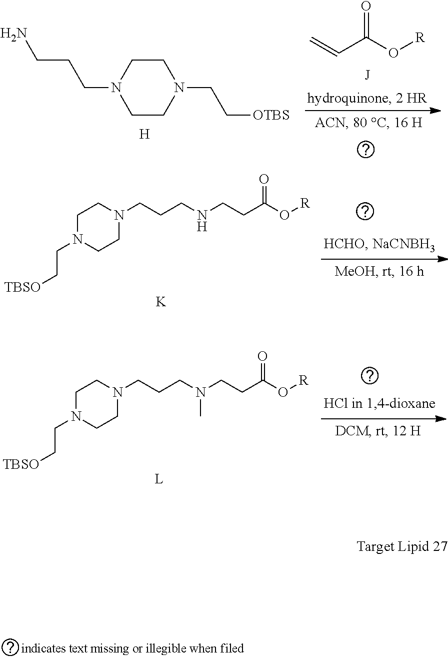

n is an integer of 1 or more
R: Linear alkyl, branched alkyl or oxycarbonyl alkyl

### Step 1: Synthesis of Compound K

To a stirred solution of compound H (1.0 eq.) in acetonitrile was added compound J (0.5 eq.) followed by the addition of hydroquinone (0.1 eq.) at room temperature and reaction mixture was stirred at 80 °C for 16 h. Progress of the reaction was monitored by TLC. After completion of the reaction, reaction mixture was concentrated under reduced pressure to afford crude compound K.

### Step 2: Synthesis of Compound L

To a stirred solution of compound K (1.0 eq.) in methanol, paraformaldehyde (10 eq.) and acetic acid (1 drop) were added and stirred at room temperature for 16 h. NaCNBH₃ (10 eq.) was added and stirred at room temperature for 16 h. Progress of the reaction was monitored by TLC. After completion of reaction, the reaction mixture was diluted with water and extracted with dichloromethane (twice). Combined organic layer was dried over anhydrous Na₂SO₄, filtered and filtrate was concentrated under reduced pressure to give crude compound. The crude compound was purified by NH-silica gel column chromatography, eluted with 20% ethyl acetate in petroleum ether to afford compound L.

### Step 3: Synthesis of Ionizable Lipid Compound 37

To a stirred solution of compound L (1.0 eq.) in dichloromethane was added 4 M HCl in 1,4-dioxane (5 eq.) at room temperature and the reaction mixture was stirred at the same temperature for 16 h. Progress of the reaction was monitored by TLC. After completion of reaction, the reaction mixture was concentrated under reduced pressure to give crude. The crude was purified by prep. HPLC to afford ionizable lipid compound 37.

### [Synthesis Formula 4] Overall Reaction Formula for Ionizable Lipid Compounds 30 to 32, 39 and 41 to 43

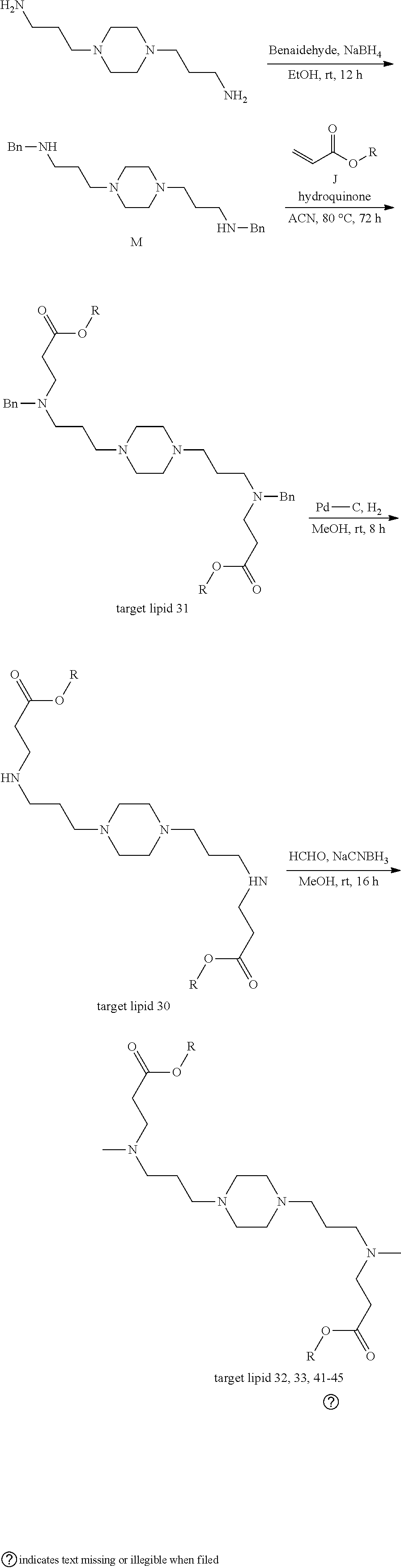

R: Linear alkyl, branched alkyl or oxycarbonyl alkyl

### Step 1: Synthesis of Compound K

To a stirred solution of 3,3′-(piperazine-1,4-diyl))bis(propan-1-amine) (1.0 eq.) in ethanol, benzaldehyde (2.1 eq.) and molecular sieves were added and stirred at room temperature (RT) for 12 h. Sodium borohydride (20 eq.) were added and stirred at RT for 12 h. Progress of the reaction was monitored by TLC. After completion of reaction, the reaction mixture was concentrated under reduced pressure to give crude residue. The residue was acidified with IN HCl and washed with dichloromethane (twice). The aqueous layer was basified with IN NaOH and extracted with dichloromethane (twice). The combined organic layer was dried over anhydrous Na₂SO₄, filtered and the filtrate was concentrated under reduced pressure to give crude compound. The crude compound was purified by neutral alumina column chromatography, eluted with 5% ethyl acetate/petroleum ether to afford compound M.

### Step 2-1: Synthesis of Ionizable Lipid Compound 31

To a stirred solution of compound J (2.5 eq.) in acetonitrile, compound M (1.0 eq.) and catalytic amount of hydroquinone (0.1 eq.) were added at room temperature. The reaction mixture was heated to 80 °C and stirred at the same temperature for 72 h. The progress of is the reaction was monitored by TLC. After completion of reaction, the reaction mixture was diluted with water and extracted with ethyl acetate. The combined organic layer was dried over anhydrous Na₂SO₄, filtered and the filtrate was concentrated under reduced pressure to give crude compound. The crude compound was purified by silica gel (100-200 mesh) column chromatography, eluted with 5% methanol/dichloromethane to afford ionizable lipid compound 31.

### Step 2-2: Synthesis of Ionizable Lipid Compound 30

To a stirred solution of ionizable lipid compound 31 (1.0 eq.) in methanol, Pd/C (0.1 eq.) was added and stirred under hydrogen gas balloon pressure (60 psi) for 8 h. Progress of the reaction was monitored by TLC. After completion of reaction, the reaction mixture was filtered through celite and washed with methanol. The filtrate was concentrated under reduced pressure to afford crude compound. Crude was purified by prep. HPLC to afford ionizable lipid compound 30.

### Step 2-3: Synthesis of Ionizable Lipid Compounds 32, 39, and 41 to 43

To a stirred solution of ionizable lipid compound 30 (1.0 eq.) in methanol, paraformaldehyde (10 eq.) and acetic acid (1 drop) were added and stirred at RT for 4 h. NaCNBH₃ (10 eq.) was added and stirred at RT for 16 h. Progress of the reaction was monitored by TLC. After completion of reaction, the reaction mixture was diluted with water and extracted with dichloromethane (twice). The combined organic layer was dried over anhydrous Na₂SO₄, filtered and the filtrate was concentrated under reduced pressure to give crude compound. The crude compound was purified by prep. HPLC to afford ionizable lipid compounds 32, 39, 41-43.

### [Synthesis Formula 5] Overall Reaction Formula for Ionizable Lipid Compounds 38

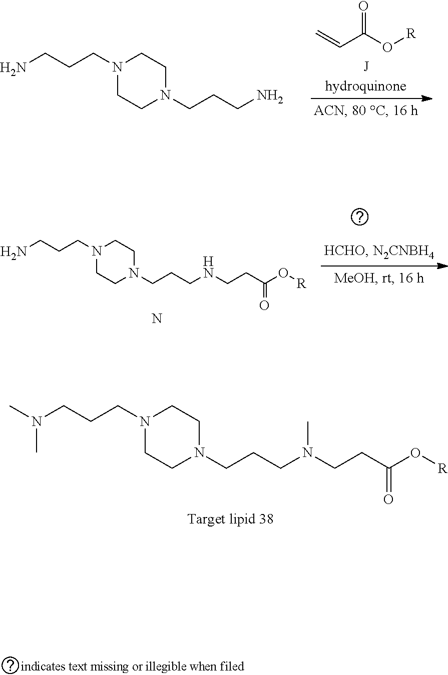

R: Linear alkyl, branched alkyl or oxycarbonyl alkyl

### Step 1: Synthesis of Compound N

To a stirred solution of 3,3′-(piperazine-1,4-diyl)bis(propan-1-amine) (1.0 eq.) in acetonitrile was added compound J (0.8 eq.) followed by the addition of hydroquinone (0.1 eq.) at room temperature and reaction mixture was stirred at 80 °C for 16 h. Progress of the reaction was monitored by TLC. After completion of the reaction, the reaction mixture was concentrated under reduced pressure to give compound N.

### Step 2: Synthesis of Ionizable Lipid Compound 38

To a stirred solution of compound N (1.0 eq.) in methanol was added formaldehyde (10.0 eq.) followed by the addition of acetic acid (1 drop) at room temperature and reaction mixture was stirred at room temperature for 1 h. NaCNBH₃ (10.0 eq.) was added and stirred at room temperature for 16 h. Progress of the reaction was monitored by TLC. After completion of reaction, the reaction mixture was diluted with water and extracted with 10% methanol in dichloromethane (twice). Combined organic layer was washed with water, brine, dried over anhydrous sodium sulfate, filtered and filtrate was concentrated under reduced pressure to give crude compound. The crude compound was purified by prep-HPLC to afford ionizable lipid compound 38.

### [Synthesis Formula 6] Overall Reaction Formula for Ionizable Lipid Compound 46

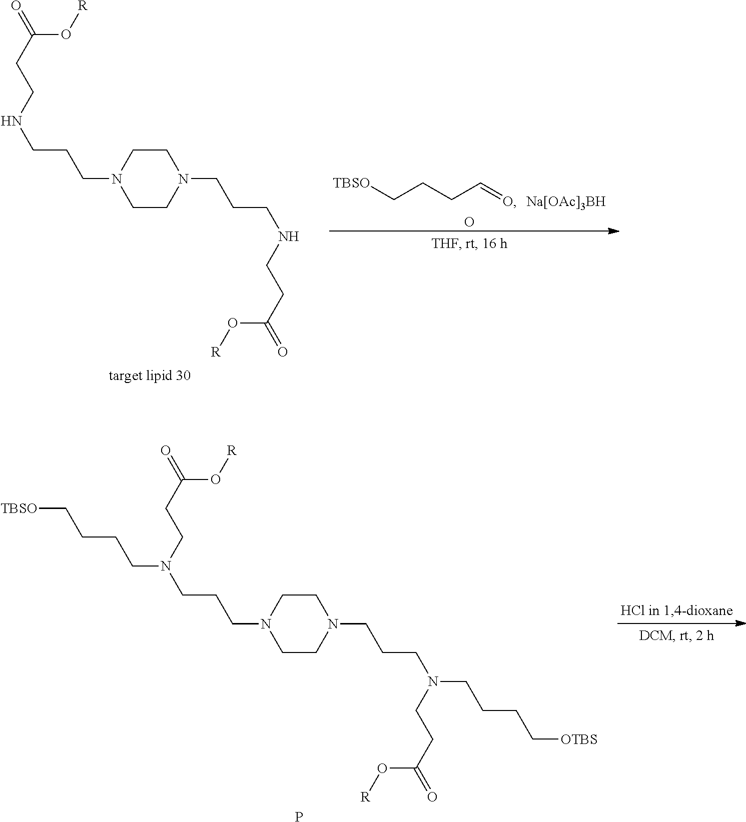

R: Linear alkyl, branched alkyl or oxycarbonyl alkyl

### Step 1: Synthesis of Compound P

To a stirred solution of ionizable lipid compound 30 (1.0 eq.) in Tetrahydrofuran(THF), compound O (2.50 eq.) and acetic acid (1 drop) were added and stirred at room temperature for 1 h. Na(OAc)₃BH (4 eq.) was added and stirred at room temperature for 16 h. Progress of the reaction was monitored by TLC. After completion of reaction, the reaction mixture was diluted with water (50 mL) and extracted with 10% methanol in dichloromethane (3*50 mL). The combined organic layer was dried over anhydrous Na₂SO₄, filtered and the filtrate was concentrated under reduced pressure to give crude compound. The crude compound was purified by NH-silica gel (100-200 mesh) column chromatography, eluted with 15% ethyl acetate/petroleum ether to afford compound P.

### Step 2-1: Synthesis of Ionizable Lipid Compound 46

To a stirred solution of compound P (1.0 eq.) in DCM, 1,4-dioxane-HCl (4M) (10 eq.) was added at 0 °C. Reaction mixture was then stirred at room temperature for 2 h. Progress of the reaction was monitored by TLC. After completion of reaction, the reaction mixture was quenched with NaHCO₃ solution (50 mL) and extracted with DCM (3*50 mL). The combined organic layer was dried over anhydrous Na₂SO₄, filtered and the filtrate was concentrated under reduced pressure to give crude compound. The crude compound was purified by prep-HPLC to afford ionizable lipid compound 46.

### [Synthesis Formula 7] Overall Reaction Formula for Ionizable Lipid Compounds 47 to 49

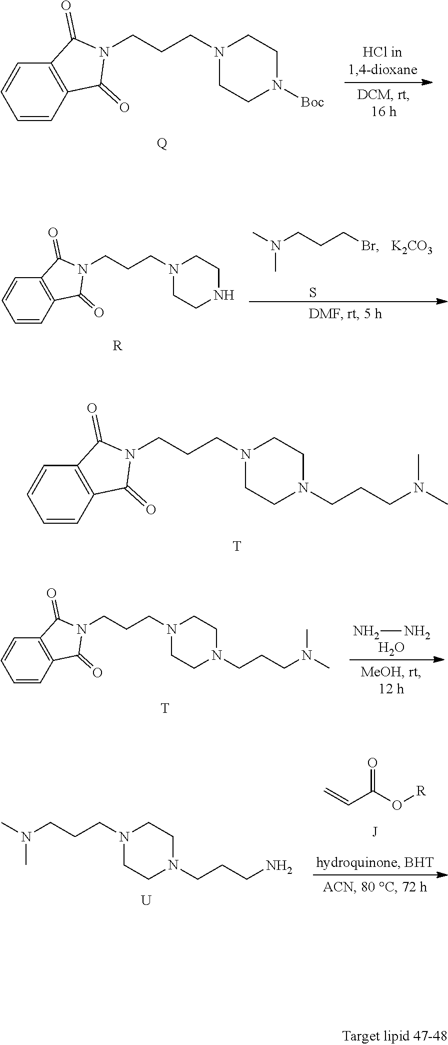

R: Linear alkyl, branched alkyl or oxycarbonyl alkyl

### Step 1: Synthesis of Compound R

To a stirred solution of compound Q (1.0 eq.) in dichloromethane was added 4 M HCl in 1,4-dioxane (5 eq.) at room temperature and the reaction mixture was stirred at the same temperature for 16 h. Progress of the reaction was monitored by TLC. After completion of reaction, the reaction mixture was concentrated under reduced pressure to give crude. The crude was purified by prep. HPLC to afford compound R.

### Step 2: Synthesis of Compound T

To a stirred solution of compound R (1.0 eq.) in DMF, K₂CO₃ (3.0 eq.) and compound S (1.2 eq.) were added and stirred at room temperature for 5 h. Progress of the reaction was monitored by TLC. After completion of reaction, the reaction mixture was diluted with water and extracted with ethyl acetate (twice). The combined organic layer was dried over anhydrous Na₂SO₄, filtered and the filtrate was concentrated under reduced pressure to give crude compound. The crude compound was purified by silica gel (100-200 mesh) column chromatography, eluted with 3% ethyl acetate/petroleum ether to afford compound T.

### Step 3: Synthesis of Compound U

To a stirred solution of compound T (1.0 eq.) in methanol, hydrazine hydrate (10 eq.) was added and stirred at room temperature for 12 h. Progress of the reaction was monitored by TLC. After completion of reaction, the reaction mixture was concentrated under reduced pressure to give crude compound. The crude compound was purified by silica gel (100-200 mesh) column chromatography, eluted with 4% ethyl acetate/petroleum ether to afford compound U.

### Step 4: Synthesis of Ionizable Lipid Compounds 47 to 49

To a stirred solution of compound J (2.5 eq.) in acetonitrile, compound U (1.0 eq.) and catalytic amount of hydroquinone (0.1 eq.) were added at room temperature. The reaction mixture was heated to 80 °C and stirred at the same temperature for 72 h. The progress of the reaction was monitored by TLC. After completion of reaction, the reaction mixture was diluted with water and extracted with ethyl acetate. The combined organic layer was dried over anhydrous Na₂SO₄, filtered and the filtrate was concentrated under reduced pressure to give crude compound. The crude compound was purified by silica gel (100-200 mesh) column chromatography, eluted with 500 methanol/dichloromethane to afford ionizable lipid compounds 47-49.

The structures of ionizable lipid compounds 1 to 49 synthesized through Synthetic Formulas 1 to 7 are listed in Table 1 below.

TABLE 1

| Com- | |
|---|---|
| pound | |
| Num- | |
| ber | Structure |
| Com- | (Tetrakis(2-ethylhexyl) 3,3′,3″,3′′′-((piperazine-1,4-diylbis(propane- |
| pound | 3,1-diyl))bis(azanetriyl))tetrapropionate) |
| 1 | |
| | 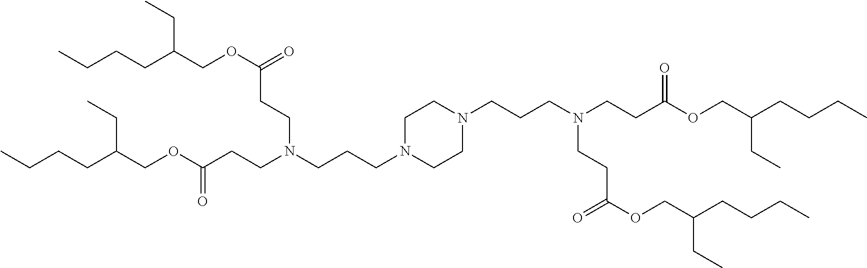 [SMILES: CCCCC(CC)COC(=O)CCN(CCCN1CCN(CCCN(CCC(=O)OCC(CC)CCCC)CCC(=O)OCC(CC)CCCC)CC1)CCC(=O)OCC(CC)CCCC] |
| Com- | (((3,3′,3″,3′″-((Piperazine-1,4-diylbis(propane-3,1- |
| pound | diyl))bis(azanetriyl))tetrakis(propanoyl))tetrakis(oxy))tetrakis(butane- |
| 2 | 4,1-diyl)tetrapentanoate) |
| | 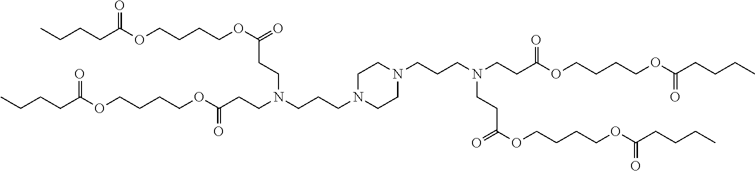 [SMILES: CCCCC(=O)OCCCCOC(=O)CCN(CCCN1CCN(CCCN(CCC(=O)OCCCCOC(=O)CCCC)CCC(=O)OCCCCOC(=O)CCCC)CC1)CCC(=O)OCCCCOC(=O)CCCC] |
| Com- | (((3,3′,3″,3″′-((Piperazine-1,4-diylbis(propane-3,1- |
| pound | diyl))bis(azanetriyl))tetrakis(propanoyl))tetrakis(oxy))tetrakis(butane- |
| 3 | 4,1-diyl)tetraheptanoate) |
| | 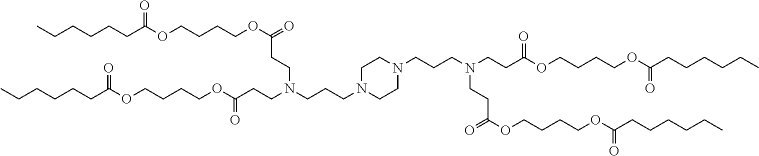 [SMILES: CCCCCCC(=O)OCCCCOC(=O)CCN(CCCN1CCN(CCCN(CCC(=O)OCCCCOC(=O)CCCCCC)CCC(=O)OCCCCOC(=O)CCCCCC)CC1)CCC(=O)OCCCCOC(=O)CCCCCC] |
| Com- | (((3,3′,3″,3″-((Piperazine-1,4-diylbis(propane-3,1- |
| pound | diyl))bis(azanetriyl))tetrakis(propanoyl))tetrakis(oxy))tetrakis(butane- |
| 4 | 4,1-diyl)tetranonanoate) |
| | 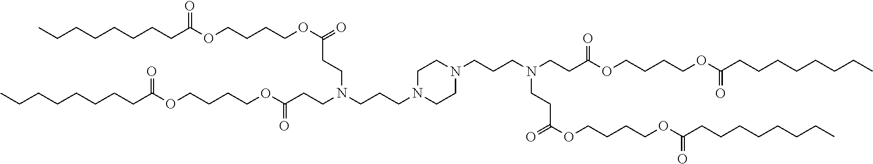 [SMILES: CCCCCCCCC(=O)OCCCCOC(=O)CCN(CCCN1CCN(CCCN(CCC(=O)OCCCCOC(=O)CCCCCCCC)CCC(=O)OCCCCOC(=O)CCCCCCCC)CC1)CCC(=O)OCCCCOC(=O)CCCCCCCC] |
| Com- | (Tetra(octan-3-yl) 3,3′,3″,3″′-((piperazine-1,4-diylbis(propane-3,1- |
| pound | diyl))bis(azanetriyl))tetrapropionate) |
| 5 | |
| | 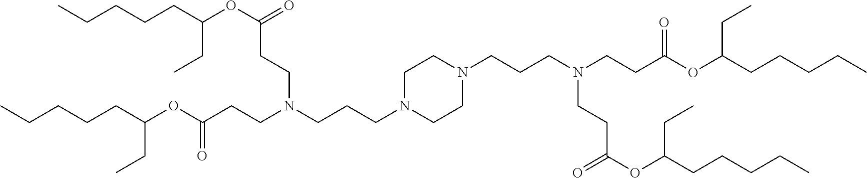 [SMILES: CCCCCC(CC)OC(=O)CCN(CCCN1CCN(CCCN(CCC(=O)OC(CC)CCCCC)CCC(=O)OC(CC)CCCCC)CC1)CCC(=O)OC(CC)CCCCC] |
| Com- | (Tetrakis(4-(2-cyclohexylacetoxy)butyl) 3,3′,3″,3″′-((piperazine- |
| pound | 1,4-diylbis(propane-3,1-diyl))bis(azanetriyl))tetrapropionate) |
| 6 | |
| | 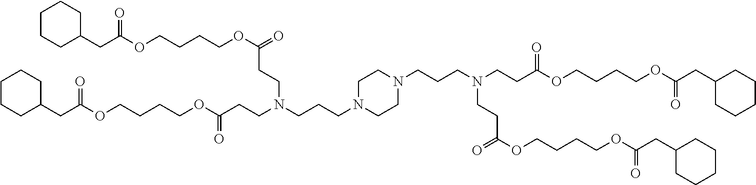 [SMILES: O=C(CCN(CCCN1CCN(CCCN(CCC(=O)OCCCCOC(=O)CC2CCCCC2)CCC(=O)OCCCCOC(=O)CC2CCCCC2)CC1)CCC(=O)OCCCCOC(=O)CC1CCCCC1)OCCCCOC(=O)CC1CCCCC1] |
| Com- | (((3,3′,3″,3′′′-((Piperazine-1,4-diylbis(propane-3,1- |
| pound | diyl))bis(azanetriyl))tetrakis(propanoyl))tetrakis(oxy))tetrakis(butane- |
| 7 | 4,1-diyl)tetraundecanoate) |
| | 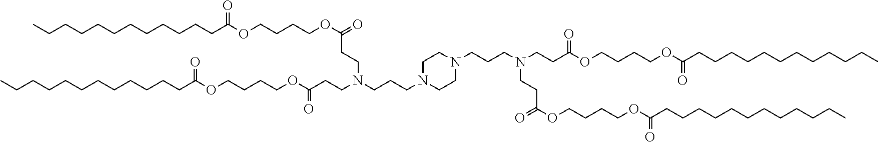 [SMILES: CCCCCCCCCCCCC(=O)OCCCCOC(=O)CCN(CCCN1CCN(CCCN(CCC(=O)OCCCCOC(=O)CCCCCCCCCCCC)CCC(=O)OCCCCOC(=O)CCCCCCCCCCCC)CC1)CCC(=O)OCCCCOC(=O)CCCCCCCCCCCC] |
| Com- | (Tetrakis(4-(propionyloxy)butyl) 3,3′,3″,3′″-((piperazine-1,4- |
| pound | diylbis(propane-3,1-diyl))bis(azanetriyl))tetrapropionate) |
| 8 | |
| | 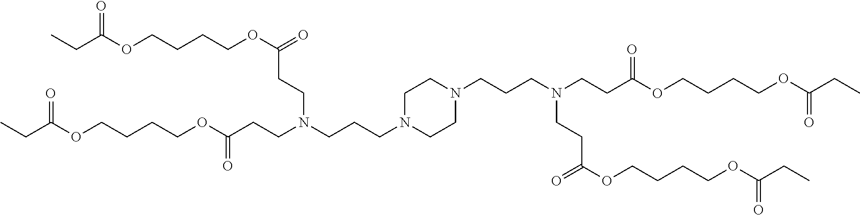 [SMILES: CCC(=O)OCCCCOC(=O)CCN(CCCN1CCN(CCCN(CCC(=O)OCCCCOC(=O)CC)CCC(=O)OCCCCOC(=O)CC)CC1)CCC(=O)OCCCCOC(=O)CC] |
| Com- | (((3,3′,3″,3′″-((Piperazine-1,4-diylbis(propane-3,1- |
| pound | diyl))bis(azanetriyl))tetrakis(propanoyl))tetrakis(oxy))tetrakis(ethane- |
| 9 | 2,1-diyl)tetraheptanoate) |
| | 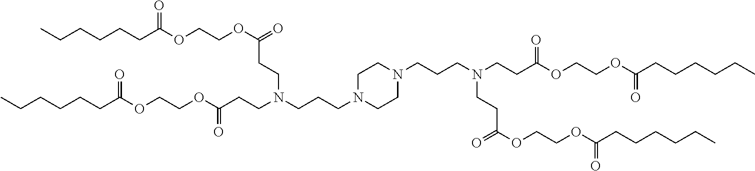 [SMILES: CCCCCCC(=O)OCCOC(=O)CCN(CCCN1CCN(CCCN(CCC(=O)OCCOC(=O)CCCCCC)CCC(=O)OCCOC(=O)CCCCCC)CC1)CCC(=O)OCCOC(=O)CCCCCC] |
| Com- | (((3,3′,3″,3′′′-((Piperazine-1,4-diylbis(propane-3,1- |
| pound | diyl))bis(azanetriyl))tetrakis(propanoyl))tetrakis(oxy))tetrakis(propane- |
| 10 | 3,1-diyl)tetrahexanoate) |
| | 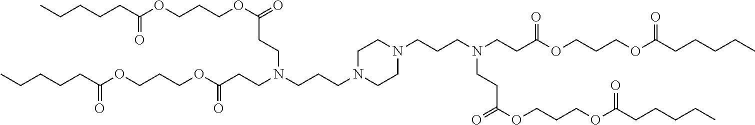 [SMILES: CCCCCC(=O)OCCCOC(=O)CCN(CCCN1CCN(CCCN(CCC(=O)OCCCOC(=O)CCCCC)CCC(=O)OCCCOC(=O)CCCCC)CC1)CCC(=O)OCCCOC(=O)CCCCC] |
| Com- | (((3,3′,3″,3′′′-((Piperazine-1,4-diylbis(propane-3,1- |
| pound | diyl))bis(azanetriyl))tetrakis(propanoyl))tetrakis(oxy))tetrakis(pentane- |
| 11 | 5,1-diyl)tetrabutyrate) |
| | 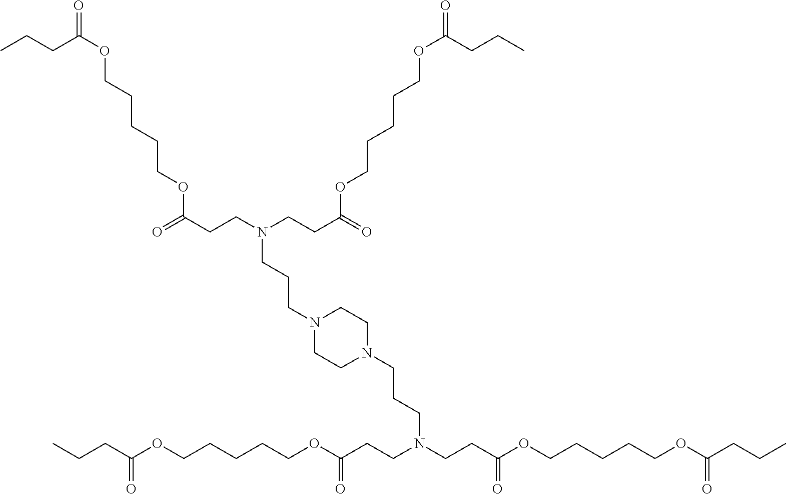 [SMILES: CCCC(=O)OCCCCCOC(=O)CCN(CCCN1CCN(CCCN(CCC(=O)OCCCCCOC(=O)CCC)CCC(=O)OCCCCCOC(=O)CCC)CC1)CCC(=O)OCCCCCOC(=O)CCC] |
| Com- | (Tetrakis(2-ethylhexyl) 4,4′,4″,4′′′-((3,3′,3″,3″′-((piperazine-1,4- |
| pound | diylbis(propane-3,1- |
| 12 | diyl))bis(azanetriyl))tetrakis(propanoyl))tetrakis(oxy))tetrabutyrate) |
| | 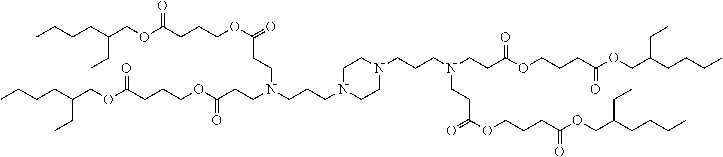 [SMILES: CCCCC(CC)COC(=O)CCCOC(=O)CCN(CCCN1CCN(CCCN(CCC(=O)OCCCC(=O)OCC(CC)CCCC)CCC(=O)OCCCC(=O)OCC(CC)CCCC)CC1)CCC(=O)OCCCC(=O)OCC(CC)CCCC] |
| Com- | (Tetrakis(2-ethylhexyl) 6,6′,6″,6′′′-((3,3′,3″,3′″-((piperazine-1,4- |
| pound | diylbis(propane-3,1- |
| 13 | diyl))bis(azanetriyl))tetrakis(propanoyl))tetrakis(oxy))tetrahexanoate) |
| | 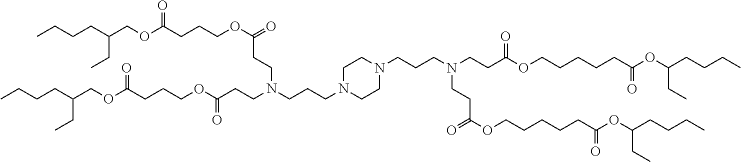 [SMILES: CCCCC(CC)COC(=O)CCCOC(=O)CCN(CCCN1CCN(CCCN(CCC(=O)OCCCCCC(=O)OC(CC)CCCC)CCC(=O)OCCCCCC(=O)OC(CC)CCCC)CC1)CCC(=O)OCCCC(=O)OCC(CC)CCCC] |
| Com- | (((3,3′,3″,3′″-((Piperazine-1,4-diylbis(propane-3,1- |
| pound | diyl))bis(azanetriyl))tetrakis(propanoyl))tetrakis(oxy))tetrakis(hexane- |
| 14 | 6,1-diyl)tetrakis(2-hexyldecanoate)) |
| | 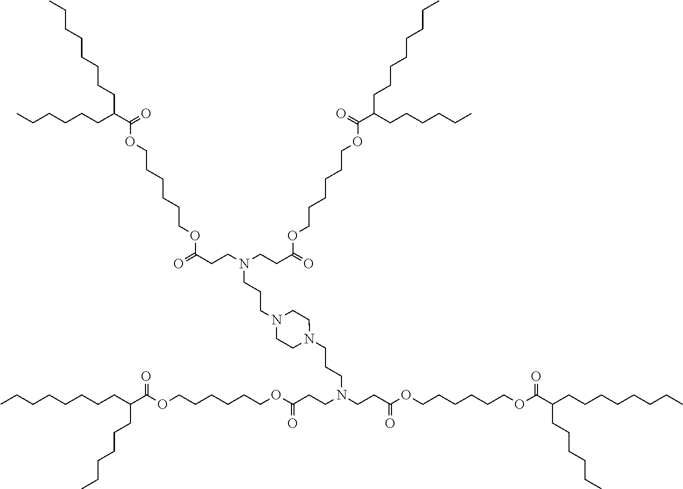 [SMILES: CCCCCCCCC(CCCCCC)C(=O)OCCCCCCOC(=O)CCN(CCCN1CCN(CCCN(CCC(=O)OCCCCCCOC(=O)C(CCCCCC)CCCCCCCC)CCC(=O)OCCCCCCOC(=O)C(CCCCCC)CCCCCCCC)CC1)CCC(=O)OCCCCCCOC(=O)C(CCCCCC)CCCCCCCC] |
| Com- | (((3,3′,3″,3′′′-((Piperazine-1,4-diylbis(propane-3,1- |
| pound | diyl))bis(azanetriyl))tetrakis(propanoyl))tetrakis(oxy))tetrakis(octane- |
| 15 | 8,1-diyl)tetrakis(2-hexyldecanoate)) |
| | 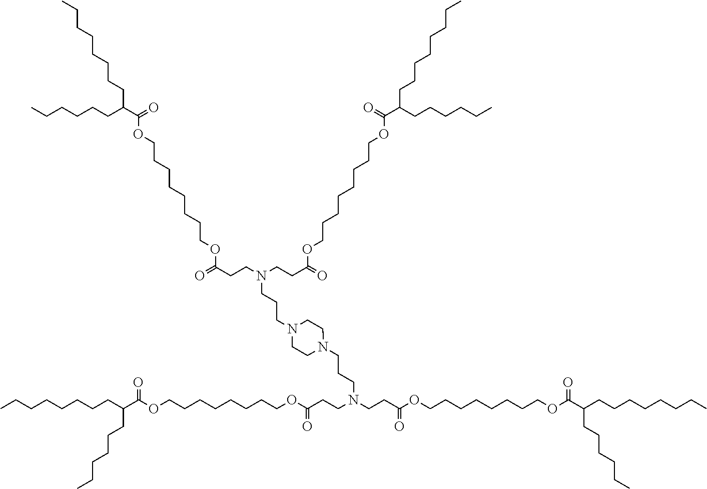 [SMILES: CCCCCCCCC(CCCCCC)C(=O)OCCCCCCCCOC(=O)CCN(CCCN1CCN(CCCN(CCC(=O)OCCCCCCCCOC(=O)C(CCCCCC)CCCCCCCC)CCC(=O)OCCCCCCCCOC(=O)C(CCCCCC)CCCCCCCC)CC1)CCC(=O)OCCCCCCCCOC(=O)C(CCCCCC)CCCCCCCC] |
| Com- | ((((3,3′,3″,3″′-((Piperazine-1,4-diylbis(propane-3,1- |
| pound | diyl))bis(azanetriyl))tetrakis(propanoyl))tetrakis(oxy))tetrakis(methylene)) |
| 16 | tetrakis(propane-2,1,3-triyl)octakis(decanoate)) |
| | 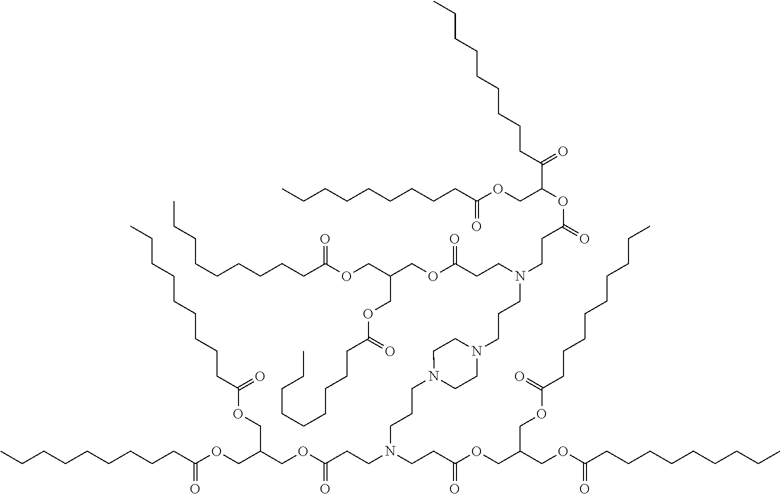 [SMILES: CCCCCCCCCC(=O)OCC(COC(=O)CCCCCCCCC)COC(=O)CCN(CCCN1CCN(CCCN(CCC(=O)OCC(COC(=O)CCCCCCCCC)COC(=O)CCCCCCCCC)CCC(=O)OC(COC(=O)CCCCCCCCC)C(=O)CCCCCCCCC)CC1)CCC(=O)OCC(COC(=O)CCCCCCCCC)COC(=O)CCCCCCCCC] |
| Com- | Tetrakis(2-ethylhexyl) 8,8′,8″,8′′′-((3,3′,3″,3′′′-((piperazine-1,4- |
| pound | diylbis(propane-3,1- |
| 17 | diyl))bis(azanetriyl))tetrakis(propanoyl))tetrakis(oxy))tetraoctanoate |
| | 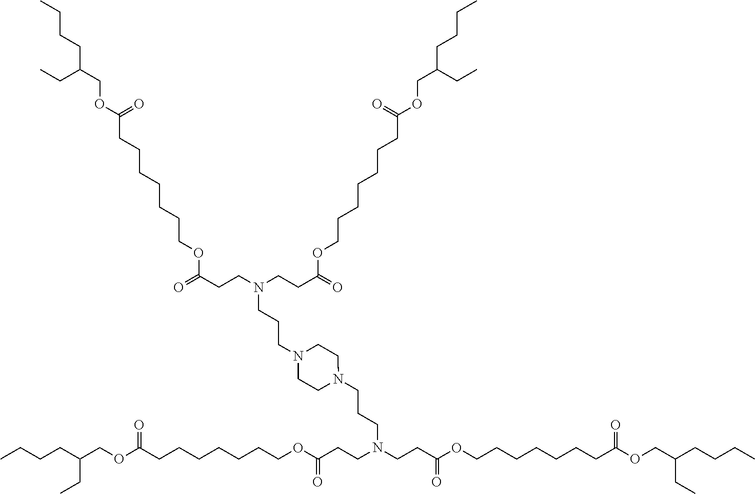 [SMILES: CCCCC(CC)COC(=O)CCCCCCCOC(=O)CCN(CCCN1CCN(CCCN(CCC(=O)OCCCCCCCC(=O)OCC(CC)CCCC)CCC(=O)OCCCCCCCC(=O)OCC(CC)CCCC)CC1)CCC(=O)OCCCCCCCC(=O)OCC(CC)CCCC] |
| Com- | ((3,3′,3″,3′″-((Piperazine-1,4-diylbis(propane-3,1- |
| pound | diyl))bis(azanetriyl))tetrakis(propanoyl))tetrakis(oxy))tetrakis(butane- |
| 18 | 4,1-diyl) tetrakis(2-hexyldecanoate) |
| | 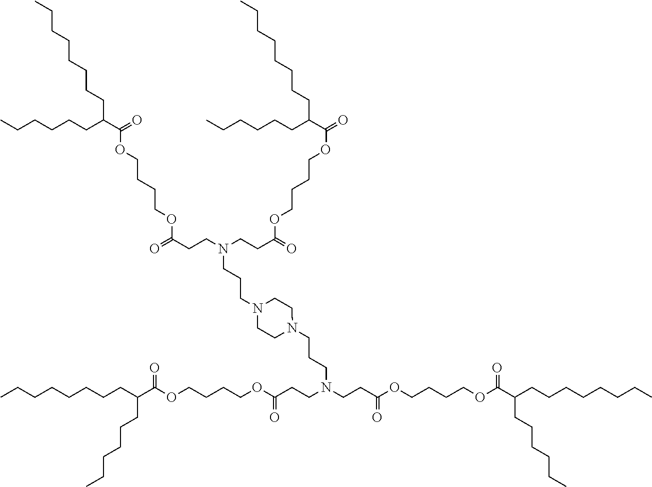 [SMILES: CCCCCCCCC(CCCCCC)C(=O)OCCCCOC(=O)CCN(CCCN1CCN(CCCN(CCC(=O)OCCCCOC(=O)C(CCCCCC)CCCCCCCC)CCC(=O)OCCCCOC(=O)C(CCCCCC)CCCCCCCC)CC1)CCC(=O)OCCCCOC(=O)C(CCCCCC)CCCCCCCC] |
| Com- | (((3,3′,3″,3′′′-((Piperazine-1,4-diylbis(propane-3,1- |
| pound | diyl))bis(azanetriyl))tetrakis(propanoyl))tetrakis(oxy))tetrakis(methylene)) |
| 19 | tetrakis(propane-2,1,3-triyl) octahexanoate |
| | 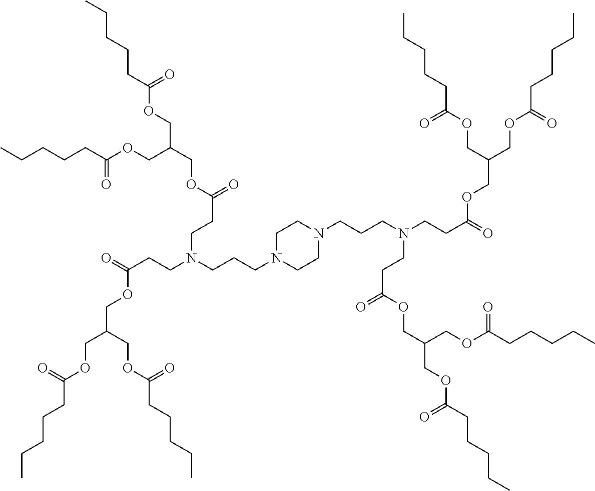 [SMILES: CCCCCC(=O)OCC(COC(=O)CCCCC)COC(=O)CCN(CCCN1CCN(CCCN(CCC(=O)OCC(COC(=O)CCCCC)COC(=O)CCCCC)CCC(=O)OCC(COC(=O)CCCCC)COC(=O)CCCCC)CC1)CCC(=O)OCC(COC(=O)CCCCC)COC(=O)CCCCC] |
| Com- | (((3,3′,3″,3′″-((Piperazine-1,4-diylbis(propane-3,1- |
| pound | diyl))bis(azanetriyl))tetrakis(propanoyl))tetrakis(oxy))tetrakis(methylene)) |
| 20 | tetrakis(propane-2,1,3-triyl) octaoctanoate |
| | 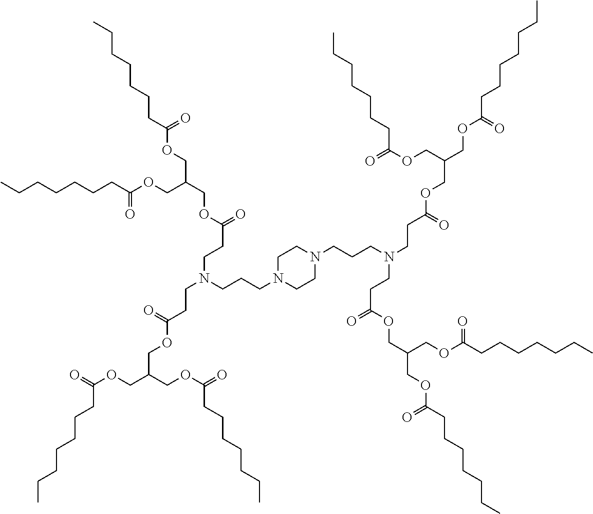 [SMILES: CCCCCCCC(=O)OCC(COC(=O)CCCCCCC)COC(=O)CCN(CCCN1CCN(CCCN(CCC(=O)OCC(COC(=O)CCCCCCC)COC(=O)CCCCCCC)CCC(=O)OCC(COC(=O)CCCCCCC)COC(=O)CCCCCCC)CC1)CCC(=O)OCC(COC(=O)CCCCCCC)COC(=O)CCCCCCC] |
| Com- | Tetrakis(2-propylhexyl) 4,4′,4″,4′′′-((3,3′,3″,3′′′-((piperazine-1,4- |
| pound | diylbis(propane-3,1- |
| 21 | diyl))bis(azanetriyl))tetrakis(propanoyl))tetrakis(oxy))tetrabutyrate |
| | 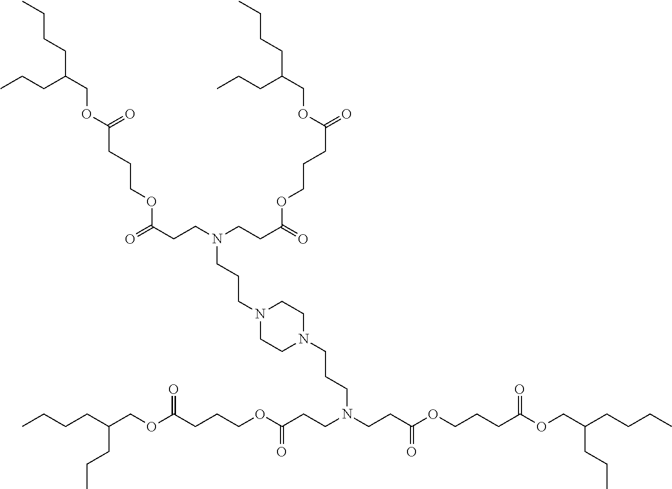 [SMILES: CCCCC(CCC)COC(=O)CCCOC(=O)CCN(CCCN1CCN(CCCN(CCC(=O)OCCCC(=O)OCC(CCC)CCCC)CCC(=O)OCCCC(=O)OCC(CCC)CCCC)CC1)CCC(=O)OCCCC(=O)OCC(CCC)CCCC] |
| Com- | Tetrakis(2-ethylpentyl) 4,4′,4″,4′′′-((3,3′,3″,3′″-((piperazine-1,4- |
| pound | diylbis(propane-3,1- |
| 22 | diyl))bis(azanetriyl))tetrakis(propanoyl))tetrakis(oxy))tetrabutyrate |
| | 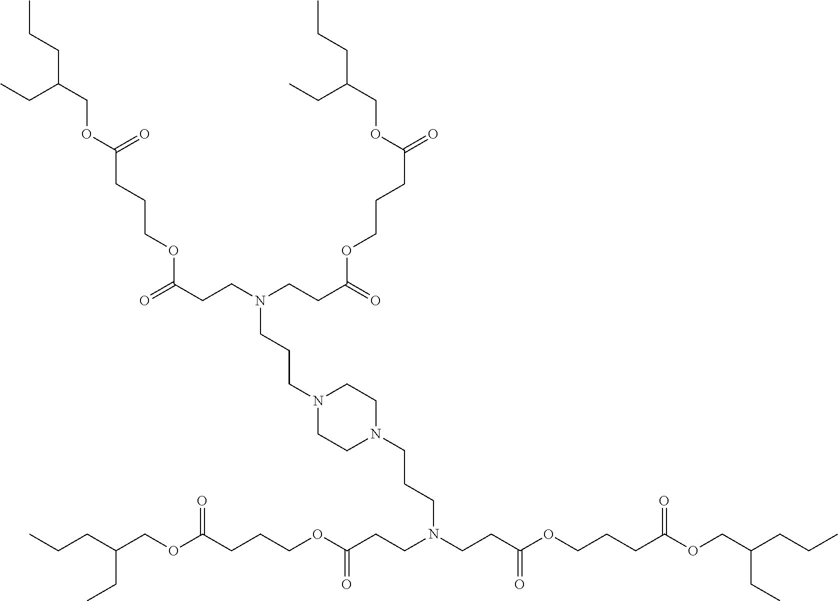 [SMILES: CCCC(CC)COC(=O)CCCOC(=O)CCN(CCCN1CCN(CCCN(CCC(=O)OCCCC(=O)OCC(CC)CCC)CCC(=O)OCCCC(=O)OCC(CC)CCC)CC1)CCC(=O)OCCCC(=O)OCC(CC)CCC] |
| Com- | Tetrakis(2-propylhexyl) 6,6′,6″,6′″-((3,3′,3″,3′′′-((piperazine-1,4- |
| pound | diylbis(propane-3,1- |
| 23 | diyl))bis(azanetriyl))tetrakis(propanoyl))tetrakis(oxy))tetrahexanoate |
| | 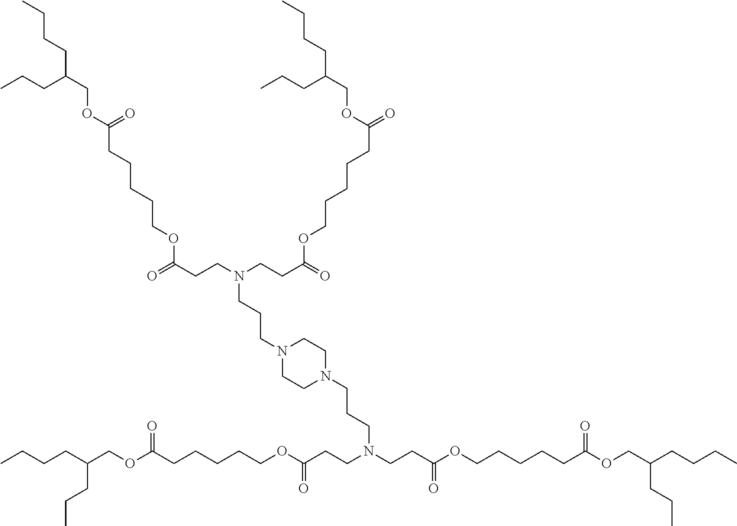 [SMILES: CCCCC(CCC)COC(=O)CCCCCOC(=O)CCN(CCCN1CCN(CCCN(CCC(=O)OCCCCCC(=O)OCC(CCC)CCCC)CCC(=O)OCCCCCC(=O)OCC(CCC)CCCC)CC1)CCC(=O)OCCCCCC(=O)OCC(CCC)CCCC] |
| Com- | Tetrakis(2-propylhexyl) 8,8′,8″,8′′′-((3,3′,3″,3′″-((piperazine-1,4- |
| pound | diylbis(propane-3,1- |
| 24 | diyl))bis(azanetriyl))tetrakis(propanoyl))tetrakis(oxy))tetraoctanoate |
| | 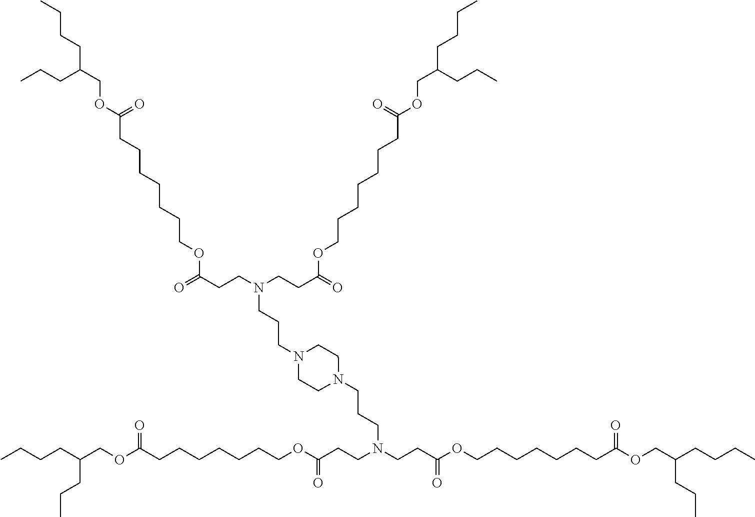 [SMILES: CCCCC(CCC)COC(=O)CCCCCCCOC(=O)CCN(CCCN1CCN(CCCN(CCC(=O)OCCCCCCCC(=O)OCC(CCC)CCCC)CCC(=O)OCCCCCCCC(=O)OCC(CCC)CCCC)CC1)CCC(=O)OCCCCCCCC(=O)OCC(CCC)CCCC] |
| Com- | Tetrakis(2-ethylpentyl) 6,6′,6″,6′′′-((3,3′,3″,3′′′-((piperazine-1,4- |
| pound | diylbis(propane-3,1- |
| 25 | diyl))bis(azanetriyl))tetrakis(propanoyl))tetrakis(oxy))tetrahexanoate |
| | 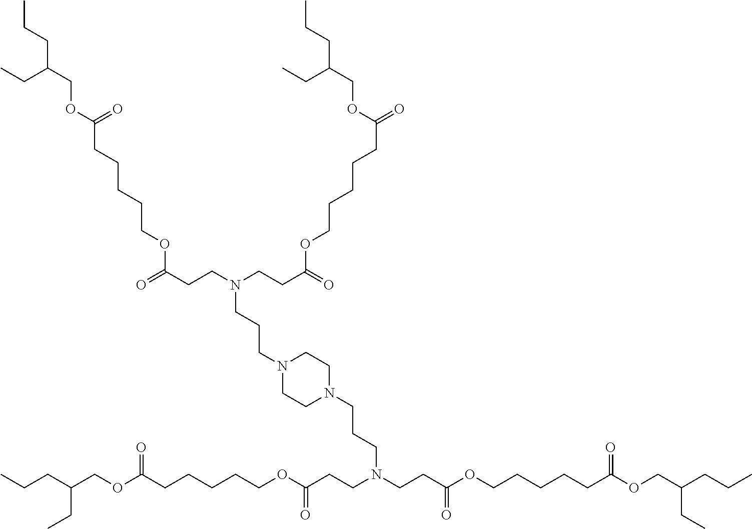 [SMILES: CCCC(CC)COC(=O)CCCCCOC(=O)CCN(CCCN1CCN(CCCN(CCC(=O)OCCCCCC(=O)OCC(CC)CCC)CCC(=O)OCCCCCC(=O)OCC(CC)CCC)CC1)CCC(=O)OCCCCCC(=O)OCC(CC)CCC] |
| Com- | Tetrakis(2-ethylpentyl) 8,8′,8″,8′′′-((3,3′,3″,3″′-((piperazine-1,4- |
| pound | diylbis(propane-3,1- |
| 26 | diyl))bis(azanetriyl))tetrakis(propanoyl))tetrakis(oxy))tetraoctanoate |
| | 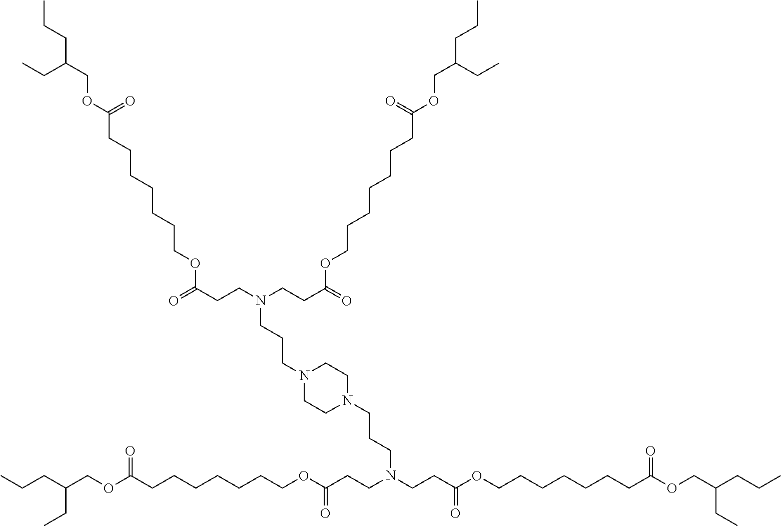 [SMILES: CCCC(CC)COC(=O)CCCCCCCOC(=O)CCN(CCCN1CCN(CCCN(CCC(=O)OCCCCCCCC(=O)OCC(CC)CCC)CCC(=O)OCCCCCCCC(=O)OCC(CC)CCC)CC1)CCC(=O)OCCCCCCCC(=O)OCC(CC)CCC] |
| Com- | Di(henicosan-10-yl) 3,3′-((3-(4-(3-hydroxypropyl)piperazin-1- |
| pound | yl)propyl)azanediyl)dipropionate |
| 27 | |
| | 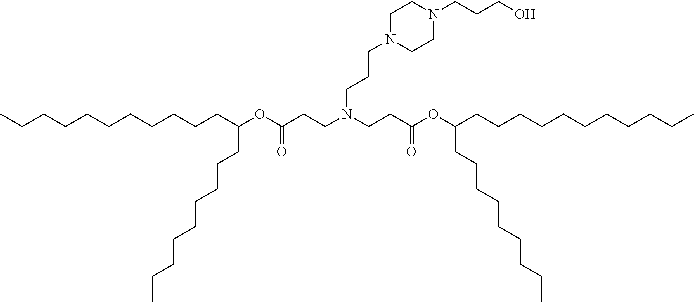 [SMILES: CCCCCCCCCCCC(CCCCCCCCC)OC(=O)CCN(CCCN1CCN(CCCO)CC1)CCC(=O)OC(CCCCCCCCC)CCCCCCCCCCC] |
| Com- | ((3,3′-((3-(4-(2-Hydroxyethyl)piperazin-1- |
| pound | yl)propyl)azanediyl)bis(propanoyl))bis(oxy))bis(hexane-6,1-diyl) |
| 28 | bis(2-hexyldecanoate) |
| | 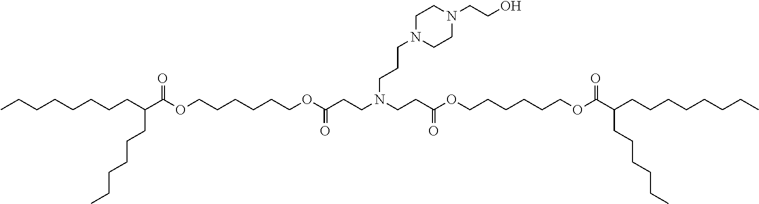 [SMILES: CCCCCCCCC(CCCCCC)C(=O)OCCCCCCOC(=O)CCN(CCCN1CCN(CCO)CC1)CCC(=O)OCCCCCCOC(=O)C(CCCCCC)CCCCCCCC] |
| Com- | ((3,3′-((3-(4-(3-Hydroxypropyl)piperazin-1- |
| pound | yl)propyl)azanediyl)bis(propanoyl))bis(oxy))bis(hexane-6,1-diyl) |
| 29 | bis(2-hexyldecanoate) |
| | 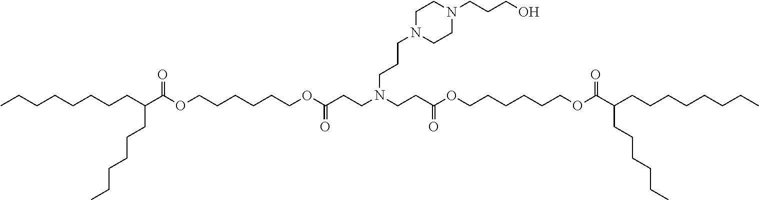 [SMILES: CCCCCCCCC(CCCCCC)C(=O)OCCCCCCOC(=O)CCN(CCCN1CCN(CCCO)CC1)CCC(=O)OCCCCCCOC(=O)C(CCCCCC)CCCCCCCC] |
| Com- | Di(henicosan-10-yl) 3,3′-((piperazine-1,4-diylbis(propane-3,1- |
| pound | diyl))bis(azanediyl))dipropionate |
| 30 | |
| | 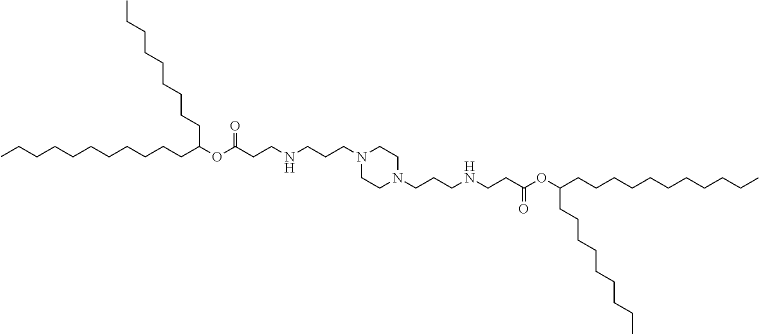 [SMILES: CCCCCCCCCCCC(CCCCCCCCC)OC(=O)CCNCCCN1CCN(CCCNCCC(=O)OC(CCCCCCCCC)CCCCCCCCCCC)CC1] |
| Com- | Di(henicosan-10-yl) 3,3′-((piperazine-1,4-diylbis(propane-3,1- |
| pound | diyl))bis(benzylazanediyl))dipropionate |
| 31 | |
| | 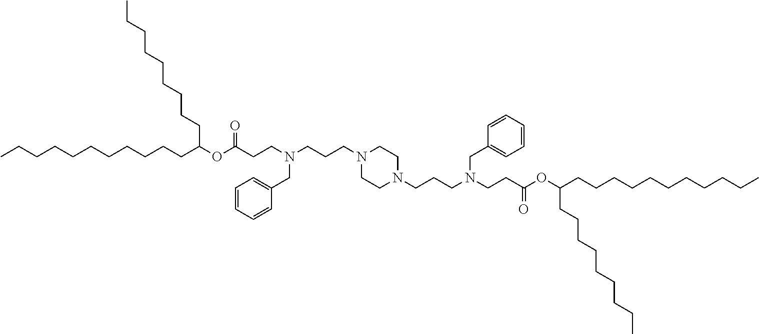 [SMILES: CCCCCCCCCCCC(CCCCCCCCC)OC(=O)CCN(CCCN1CCN(CCCN(CCC(=O)OC(CCCCCCCCC)CCCCCCCCCCC)Cc2ccccc2)CC1)Cc1ccccc1] |
| Com- | di(henicosan-10-yl) 3,3′-((piperazine-1,4-diylbis(propane-3,1- |
| pound | diyl))bis(methylazanediyl))dipropionate |
| 32 | |
| | 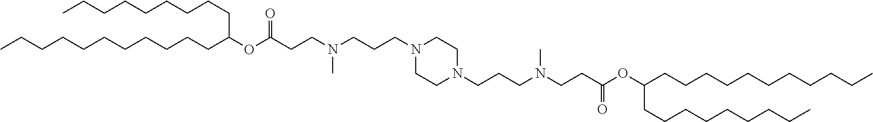 [SMILES: CCCCCCCCCCCC(CCCCCCCCC)OC(=O)CCN(C)CCCN1CCN(CCCN(C)CCC(=O)OC(CCCCCCCCC)CCCCCCCCCCC)CC1] |
| Com- | tetra((Z)-non-2-en-1-yl) 9,9′,9″,9′′′-((piperazine-1,4- |
| pound | diylbis(propane-3,1-diyl))bis(azanetriyl))tetranonanoate |
| 33 | |
| | 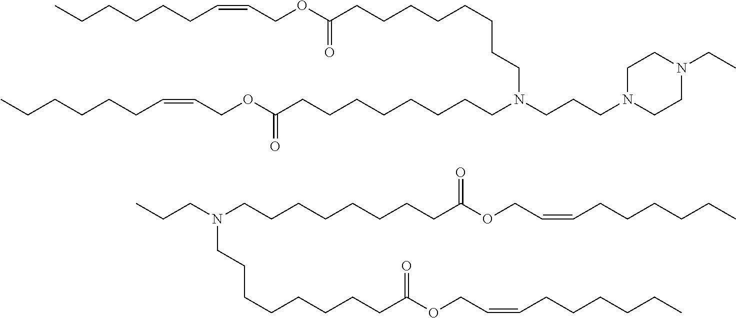 |
| Com- | tetra((Z)-non-2-en-1-yl) 6,6′,6″,6″′-((3,3′,3″,3′′′-((piperazine-1,4- |
| pound | diylbis(propane-3,1- |
| 34 | diyl))bis(azanetriyl))tetrakis(propanoyl))tetrakis(oxy))tetrahexanoate |
| | 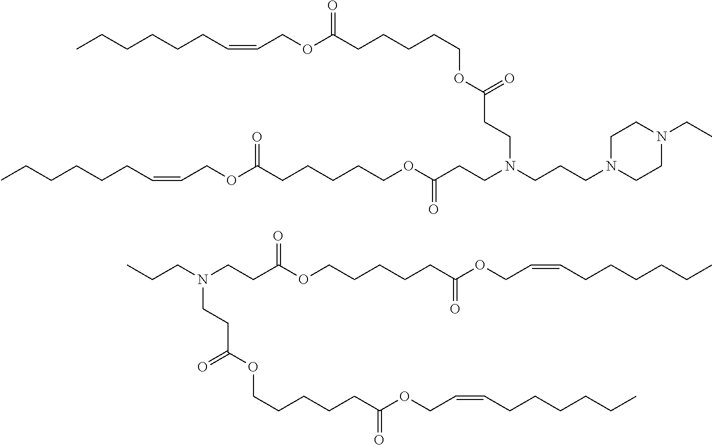 |
| Com- | tetra((Z)-non-2-en-1-yl) 4,4′,4″,4′′′-((3,3′,3″,3′″-((piperazine-1,4- |
| pound | diylbis(propane-3,1- |
| 35 | diyl))bis(azanetriyl))tetrakis(propanoyl))tetrakis(oxy))tetrabutyrate |
| | 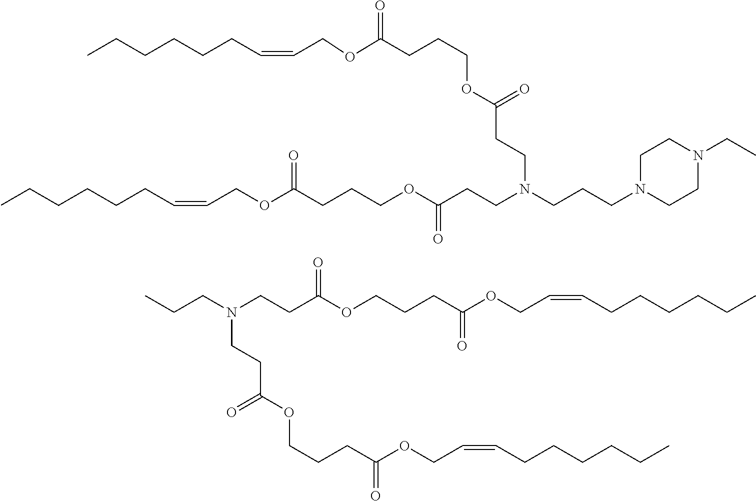 |
| Com- | di(henicosan-10-yl) 3,3′-((3-(4-(3-((3-(henicosan-10-yloxy)-3- |
| pound | oxopropyl)amino)propyl)piperazin-1-yl)propyl)azanediyl)dipropionate |
| 36 | |
| | 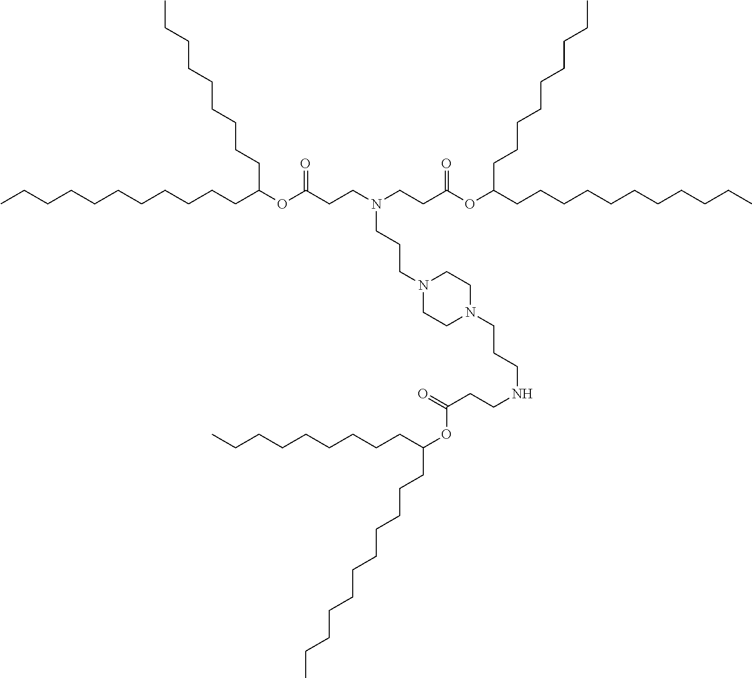 [SMILES: CCCCCCCCCCCC(CCCCCCCCC)OC(=O)CCNCCCN1CCN(CCCN(CCC(=O)OC(CCCCCCCCC)CCCCCCCCCCC)CCC(=O)OC(CCCCCCCCC)CCCCCCCCCCC)CC1] |
| Com- | (6Z,16Z)-12-((Z)-dec-4-en-1-yl)docosa-6,16-dien-11-yl 3-((3-(4-(2- |
| pound | hydroxyethyl)piperazin-1-yl)propyl)(methyl)amino)propanoate |
| 37 | |
| | 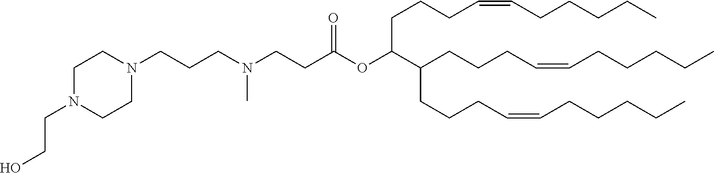 [SMILES: CCCCC/C=C\CCCC(CCC/C=C\CCCCC)C(CCC/C=C\CCCCC)OC(=O)CCN(C)CCCN1CCN(CCO)CC1] |
| Com- | (6Z,16Z)-12-((Z)-dec-4-en-1-yl)docosa-6,16-dien-11-yl 3-((3-(4-(3- |
| pound | (dimethylamino)propyl)piperazin-1- |
| 38 | yl)propyl)(methyl)amino)propanoate |
| | 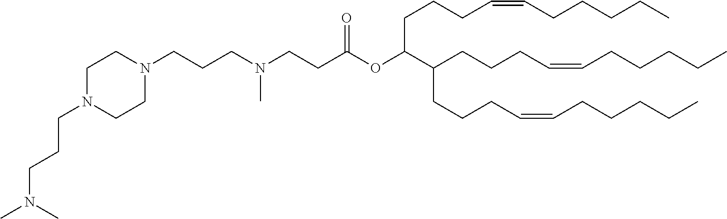 [SMILES: CCCCC/C=C\CCCC(CCC/C=C\CCCCC)C(CCC/C=C\CCCCC)OC(=O)CCN(C)CCCN1CCN(CCCN(C)C)CC1] |
| Com- | bis((6Z,16Z)-12-((Z)-dec-4-en-1-yl)docosa-6,16-dien-11-yl) 3,3′- |
| pound | ((piperazine-1,4-diylbis(propane-3,1- |
| 39 | diyl))bis(methylazanediyl))dipropionate |
| | 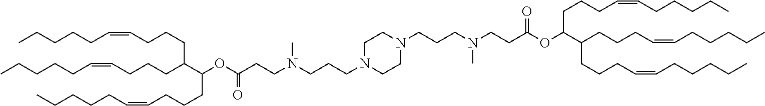 [SMILES: CCCCC/C=C\CCCC(CCC/C=C\CCCCC)C(CCC/C=C\CCCCC)OC(=O)CCN(C)CCCN1CCN(CCCN(C)CCC(=O)OC(CCC/C=C\CCCCC)C(CCC/C=C\CCCCC)CCC/C=C\CCCCC)CC1] |
| Com- | tetrakis(2-(2-cyclohexylacetoxy)ethyl) 3,3′,3″,3′′′-((piperazine-1,4- |
| pound | diylbis(propane-3,1-diyl))bis(azanetriyl))tetrapropionate |
| 40 | |
| | 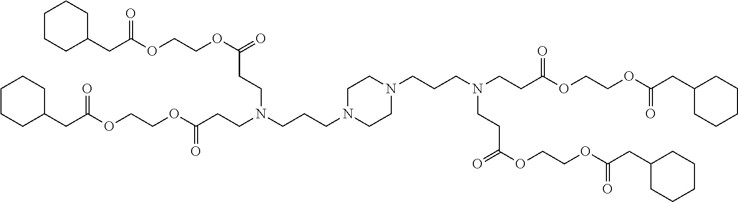 [SMILES: O=C(CCN(CCCN1CCN(CCCN(CCC(=O)OCCOC(=O)CC2CCCCC2)CCC(=O)OCCOC(=O)CC2CCCCC2)CC1)CCC(=O)OCCOC(=O)CC1CCCCC1)OCCOC(=O)CC1CCCCC1] |
| Com- | bis(4-(2-cyclohexylacetoxy)butyl) 3,3′-((piperazine-1,4- |
| pound | diylbis(propane-3,1-diyl))bis(methylazanediyl))dipropionate |
| 41 | |
| | 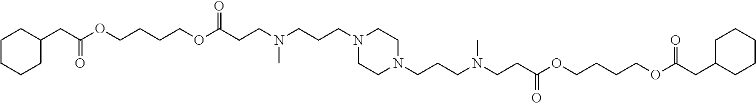 [SMILES: CN(CCCN1CCN(CCCN(C)CCC(=O)OCCCCOC(=O)CC2CCCCC2)CC1)CCC(=O)OCCCCOC(=O)CC1CCCCC1] |
| Com- | (((3,3′-((piperazine-1,4-diylbis(propane-3,1- |
| pound | diyl))bis(methylazanediyl))bis(propanoyl))bis(oxy))bis(methylene))bis |
| 42 | (propane-2,1,3-triyl)tetrahexanoate |
| | 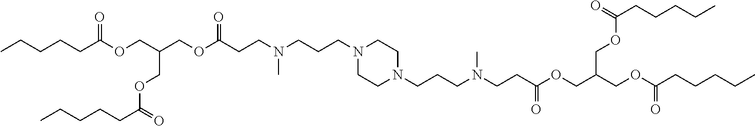 [SMILES: CCCCCC(=O)OCC(COC(=O)CCCCC)COC(=O)CCN(C)CCCN1CCN(CCCN(C)CCC(=O)OCC(COC(=O)CCCCC)COC(=O)CCCCC)CC1] |
| Com- | di(octan-3-yl) 3,3′-((piperazine-1,4-diylbis(propane-3,1- |
| pound | diyl))bis(methylazanediyl))dipropionate |
| 43 | |
| | 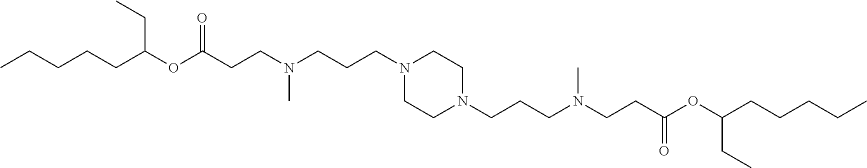 [SMILES: CCCCCC(CC)OC(=O)CCN(C)CCCN1CCN(CCCN(C)CCC(=O)OC(CC)CCCCC)CC1] |
| Com- | (((3,3′-((piperazine-1,4-diylbis(propane-3,1-diyl))bis((3-(octan-3- |
| pound | yloxy)-3- |
| 44 | oxopropyl)azanediyl))bis(propanoyl))bis(oxy))bis(methylene))bis |
| | (propane-2,1,3-triyl) tetrahexanoate |
| | 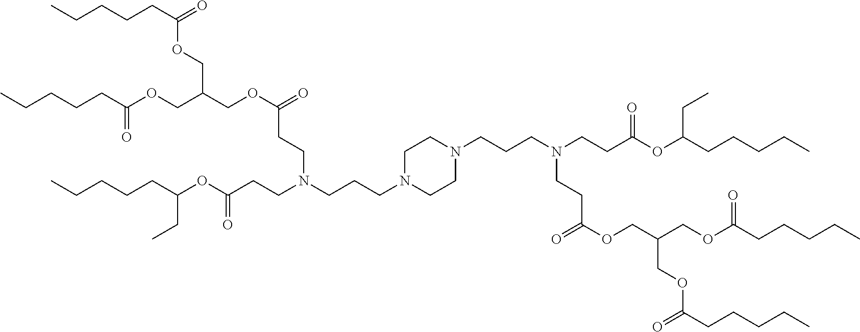 [SMILES: CCCCCC(=O)OCC(COC(=O)CCCCC)COC(=O)CCN(CCCN1CCN(CCCN(CCC(=O)OCC(COC(=O)CCCCC)COC(=O)CCCCC)CCC(=O)OC(CC)CCCCC)CC1)CCC(=O)OC(CC)CCCCC] |
| Com- | ((3,3′,3″,3′″-((piperazine-1,4-diylbis(propane-3,1- |
| pound | diyl))bis(azanetriyl))tetrakis(propanoyl))tetrakis(oxy))tetrakis(butane- |
| 45 | 4,1-diyl) tetrakis(3-ethylpentanoate) |
| | 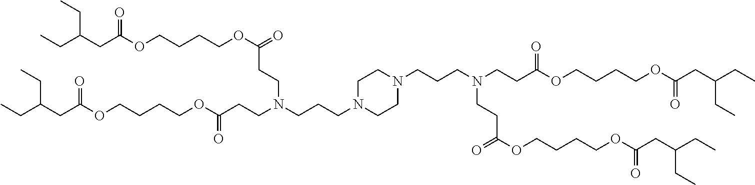 [SMILES: CCC(CC)CC(=O)OCCCCOC(=O)CCN(CCCN1CCN(CCCN(CCC(=O)OCCCCOC(=O)CC(CC)CC)CCC(=O)OCCCCOC(=O)CC(CC)CC)CC1)CCC(=O)OCCCCOC(=O)CC(CC)CC] |
| Com- | di(henicosan-10-yl) 3,3′-((piperazine-1,4-diylbis(propane-3,1- |
| pound | diyl))bis((4-hydroxybutyl)azanediyl))dipropionate |
| 46 | |
| | 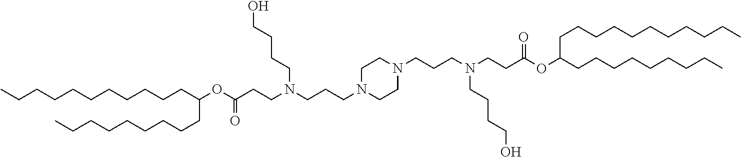 [SMILES: CCCCCCCCCCCC(CCCCCCCCC)OC(=O)CCN(CCCCO)CCCN1CCN(CCCN(CCCCO)CCC(=O)OC(CCCCCCCCC)CCCCCCCCCCC)CC1] |
| Com- | bis(2-ethylhexyl) 3,3′-((3-(4-(3-(dimethylamino)propyl)piperazin-1- |
| pound | yl)propyl)azanediyl)dipropionate |
| 47 | |
| | 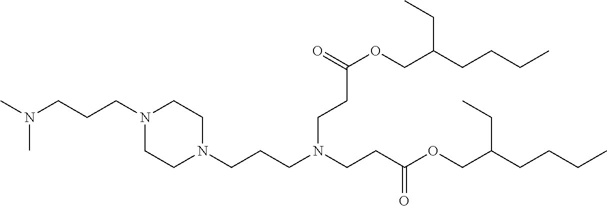 [SMILES: CCCCC(CC)COC(=O)CCN(CCCN1CCN(CCCN(C)C)CC1)CCC(=O)OCC(CC)CCCC] |
| Com- | ((3-(4-(3-(dimethylamino)propyl)piperazin-1- |
| pound | yl)propyl)azanediyl)bis(hexane-6,1-diyl)bis(2-hexyldecanoate) |
| 48 | |
| | 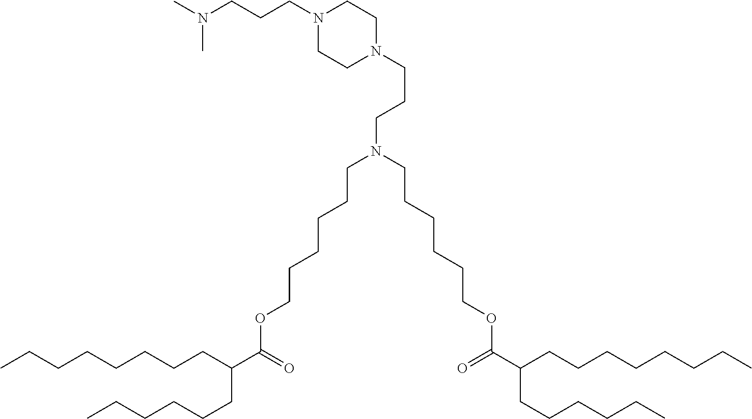 [SMILES: CCCCCCCCC(CCCCCC)C(=O)OCCCCCCN(CCCCCCOC(=O)C(CCCCCC)CCCCCCCC)CCCN1CCN(CCCN(C)C)CC1] |
| Com- | di((Z)-non-2-en-1-yl) 9,9′-((3-(4-(3- |
| pound | (dimethylamino)propyl)piperazin-1-yl)propyl)azanediyl)dinonanoate |
| 49 | |
| | 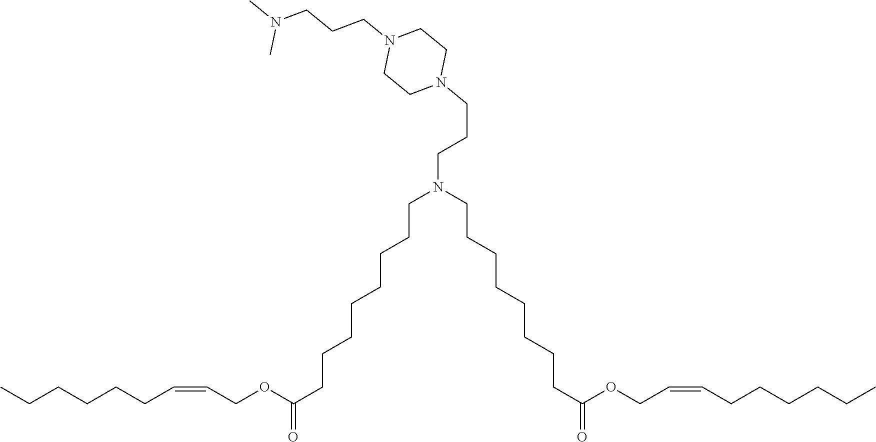 [SMILES: CCCCCC/C=C\COC(=O)CCCCCCCCN(CCCCCCCCC(=O)OC/C=C\CCCCCC)CCCN1CCN(CCCN(C)C)CC1] |

Meanwhile, among the synthesized ionizable lipids, ¹H NMR spectra were measured for compounds 1 to 49 using either a BrukerUltrashield 400 or a spectrometer. Chemical transitions were expressed in parts per million (ppm, δ units). The coupling constant is in Hertz (Hz), and the splitting pattern accounts for the variety, and is displayed by using s (singlet), d (doublet), t (triplet), q (quartet), quint (quintet), m (multiplet) or br (broad). ¹H NMR (400 MHz, CDCl₃) data of compounds 1 to 49 are shown in Table 2.

TABLE 2

| Compound | |
|---|---|
| Number | ¹H NMR (400 MHz, CDCl₃) |
| Compound 1 | δ 0.87-0.91 (m, 24H), 1.25-1.40 (m, 32H), 1.53-1.66 (m, |
| | 8H), 2.30-2.47 (m, 24H), 2.77 (t, J = 7.2 Hz, 8H), 3.94-4.02 |
| | (m, 8H); LCMS (ESI) m/z 938 [M + H]⁺. |
| Compound 2 | δ 0.92 (t, J = 7.4 Hz, 12H), 1.30-1.39 (m, 8H), 1.57-1.72 |
| | (m, 28H), 2.29-2.45 (m, 32H), 2.76 (t, J = 7.3 Hz, 8H), 4.08- |
| | 4.11 (m, 16H); LCMS (ESI) m/z 1113 [M + H]⁺ |
| Compound 3 | δ 0.87-0.90 (m, 12H), 1.25-1.33 (m, 24H), 1.58-1.73 (m, |
| | 28H), 2.28-2.46 (m, 32H), 2.76 (t, J = 7.3 Hz, 8H), 4.07-4.11 |
| | (m, 16H); LCMS (ESI) m/z 1225 [M + H]⁺ |
| Compound 4 | δ 0.86-0.9 (m, 12H), 1.26-1.32 (m, 40H), 1.60-1.71 (m, |
| | 28H), 2.28-2.45 (m, 32H), 2.76 (t, J = 7.3 Hz, 8H), 4.05-4.13 |
| | (m, 16H); LCMS (ESI) m/z 1138 [M + H]⁺ |
| Compound 5 | δ 0.85-0.89 (m, 24H), 1.23-1.34 (m, 24H), 1.48-1.63 (m, |
| | 20H), 2.29-2.66 (m, 24H), 2.77 (t, J = 7.3 Hz, 8H), 4.78-4.84 |
| | (m, 4H); LCMS (ESI) m/z 938 [M + H]⁺ |
| Compound 6 | δ 0.91-1.01 (m, 8H), 1.09-1.33 (m, 16H), 1.57-1.82 (m, |
| | 40H), 2.10-2.63 (m, 32H), 2.76 (t, J = 7.2 Hz, 8H), 4.03-4.15 |
| | (m, 16H); LCMS (ESI) m/z 1273 [M + H]⁺ |
| Compound 7 | δ 0.86-0.90 (m, 12H), 1.26-1.30 (m, 60H), 1.60-1.71 (m, |
| | 28H), 2.27-2.75 (m, 36H), 4.08-4.10 (m, 16H); LCMS (ESI) m/z |
| | 1450 [M + H]⁺ |
| Compound 8 | δ 1.14 (t, J = 7.63 Hz, 12H), 1.67-1.73 (m, 20H), 2.30- |
| | 2.76 (m, 40H), 4.08-4.11 (m, 16H); LCMS (ESI) m/z 1001 [M + H]⁺ |
| Compound 9 | δ 0.87-0.90 (m, 12H), 1.30-1.33 (m, 24H), 1.60-1.64 (m, |
| | 12H), 2.31-2.48 (m, 32H), 2.77 (t, J = 7.25 Hz, 8H), 4.27 (s, |
| | 16H); LCMS (ESI) m/z 1113 [M + H]⁺ |
| Compound 10 | δ 0.91 (t, J = 4.40 Hz, 12H), 1.26-1.36 (m, 16H), 1.59- |
| | 1.66 (m, 12H), 1.93-2.00 (m, 8H), 2.28-2.48 (m, 32H), 2.76 |
| | (t, J = 7.25 Hz, 8H), 4.15 (t, J = 6.38 Hz, 16H); LCMS (ESI) |
| | m/z 1113 [M + H]⁺ |
| Compound 11 | δ 0.95 (t, J = 7.50 Hz, 12H), 1.39-1.47 (m, 8H), 1.61-1.68 |
| | (m, 28H), 2.26-2.45 (m, 32H), 2.76 (t, J = 7.25 Hz, 8H), 4.05- |
| | 4.09 (m, 16H); LCMS (ESI) m/z 1113 [M + H]⁺ |
| Compound 12 | ¹H NMR (400 MHz, CDCl₃) δ 0.87-0.91 (m, 24H), 1.3-1.39 (m, |
| | 36H), 1.54-1.57 (m, 4H), 1.93-2.00 (m, 8H), 2.38-2.77 (m, |
| | 40H), 3.96-4.02 (m, 8H), 4.13 (t, J = 6.38 Hz, 8H); Direct |
| | MS (ESI) m/z 1281 [M + H]⁺ |
| Compound 13 | ¹H NMR (400 MHz, CDCl₃) δ 0.87-0.91 (m, 24H), 1.28-1.39 (m, |
| | 42H), 1.53-1.68 (m, 22H), 2.30-2.75 (m, 40H), 3.99 (dd, J = |
| | 5.88, 2.63 Hz, 8H), 4.05 (t, J = 6.75 Hz, 8H). Direct MS (ESI) |
| | m/z 1394 [M + H]⁺ |
| Compound 14 | δ 0.88-0.92 (m, 24H), 1.29-1.47 (m, 112H), 1.56-1.68 (m, |
| | 20H), 2.32-2.77 (m, 36H), 4.06-4.11 (m, 16H); LCMS (ESI) m/z |
| | 1842 [M + H]⁺ |
| Compound 15 | δ 0.86-0.89 (m, 24H), 1.25-1.45 (m, 120H), 1.56-1.63 (m, |
| | 28H), 2.28-3.10 (m, 36H), 4.04-4.08 (m, 16H); LCMS (ESI) m/z |
| | 1954 [M + H]⁺ |
| Compound 16 | δ 0.88 (t, J = 7.20 Hz, 24H), 1.26-1.33 (m, 88H), 1.57- |
| | 1.63 (m, 28H), 2.31 (t, J = 8.00 Hz, 20H), 2.36-2.46 (m, 22H), |
| | 2.76 (t, J = 7.20 Hz, 8H), 4.12 (d, J = 6.40 Hz, 24H); LCMS |
| | (ESI) m/z 2074 [M + H]⁺ |
| Compound 17 | ¹H NMR (400 MHz, CDCl₃) δ 0.87-0.91 (m, 24H), 1.26-1.39 (m, |
| | 56H), 1.53-1.90 (m, 24H), 2.30 (t, J = 7.6 Hz, 8H), 2.51-3.30 |
| | (m, 32H), 3.95-4.05 (m, 8H), 4.05 (t, J = 6.8 Hz, 8H); LCMS |
| | (ESI) m/z 1506 [M + H]⁺ |
| Compound 18 | ¹H NMR (400 MHz, CDCl₃) δ 0.86-0.89 (m, 24H), 1.25-1.29 (m, |
| | 84H), 1.40-1.45 (m, 8H), 1.56-1.59 (m, 12H), 1.69-1.71 (m, |
| | 16H), 2.29-2.45 (m, 24H), 2.76 (t, J = 7.2 Hz, 8H), 4.09-4.10 |
| | (m, 16H); LCMS (ESI) m/z 1730 [M + H]⁺ |
| Compound 19 | ¹H NMR (400 MHz, CDCl₃) δ 0.90 (t, J = 7.2 Hz, 24H), 1.27- |
| | 1.35 (m, 32H), 1.60-1.66 (m, 20H), 2.28-2.70 (m, 42H), 2.76 |
| | (t, J = 6.0 Hz, 8H), 4.13 (d, J = 6.0 Hz, 24H); LCMS (ESI) |
| | m/z 1626 [M + H]⁺ |
| Compound 20 | ¹H NMR (400 MHz, CDCl₃) δ 0.88 (t, J = 6.8 Hz, 24H), 1.21- |
| | 1.35 (m, 64H), 1.59-1.69 (m, 20H), 2.20-2.60 (m, 44H), 2.76 |
| | (t, J = 7.2 Hz, 8H), 2.88-3.02 (m, 4H), 4.12 (d, J = 6.4 Hz, |
| | 24H); LCMS (ESI) m/z 1850 [M + H]⁺ |
| Compound 21 | ¹H NMR (400 MHz, CDCl₃) δ 0.88-0.91 (m, 24H), 1.24-1.36 (m, |
| | 40H), 1.63-1.65 (m, 8H), 1.96 (quintet, J = 7.2 Hz, 8H), 2.38- |
| | 3.20 (m, 40H), 3.99 (d, J = 6.0 Hz, 8H), 4.10 (t, J = 6.4 Hz, |
| | 8H); LCMS (ESI) m/z 1337 [M + H]⁺ |
| Compound 22 | ¹H NMR (400 MHz, CDCl₃) δ 0.89-0.92 (m, 24H), 1.26-1.37 (m, |
| | 24H), 1.57-1.62 (m, 8H), 1.94-1.98 (m, 8H), 2.30-2.46 (m, |
| | 32H), 2.76 (t, J = 7.6 Hz, 8H), 3.95-4.05 (m, 8H), 4.10 (t, |
| | J = 6.5 Hz, 8H); LCMS (ESI) m/z 1225 [M + H]⁺ |
| Compound 23 | ¹H NMR (400 MHz, CDCl₃) δ 0.85-0.93 (m, 24H), 1.24-1.42 (m, |
| | 52H), 1.60-1.70 (m, 20H), 2.29-2.48 (m, 32H), 2.74-2.78 (m, |
| | 8H), 3.97 (d, J = 6.0 Hz, 8H), 4.05 (t, J = 6.8 Hz, 8H); LCMS |
| | (ESI) m/z 1449 [M + H]⁺ |
| Compound 24 | ¹H NMR (400 MHz, CDCl₃) δ 0.88-0.91 (m, 24H), 1.26-1.43 (m, |
| | 64H), 1.60-1.62 (m, 24H), 2.28-2.76 (m, 40H), 3.97 (d, J = |
| | 5.6 Hz, 8H), 4.04 (t, J = 6.8 Hz, 8H); LCMS (ESI) m/z 1562 |
| | [M + H]⁺ |
| Compound 25 | ¹H NMR (400 MHz, CDCl₃) δ 0.87-0.92 (m, 24H), 1.26-1.40 (m, |
| | 32H), 1.58-1.68 (m, 24H), 2.31-2.70 (m, 32H), 2.75 (t, J = |
| | 7.2 Hz, 8H), 3.95-4.04 (m, 8H), 4.05 (t, J = 6.8 Hz, 8H); |
| | LCMS (ESI) m/z 1338 [M + H]⁺ |
| Compound 26 | ¹H NMR (400 MHz, CDCl₃) δ 0.85-0.95 (m, 24H), 1.25-1.43 (m, |
| | 48H), 1.55-1.62 (m, 24H), 2.28-2.77 (m, 40H), 3.95-4.03 (m, |
| | 8H), 4.04 (t, J = 6.8 Hz, 8H); LCMS (ESI) m/z 1450 [M + H]⁺ |
| Compound 27 | ¹H NMR (400 MHz, CDCl₃) δ 0.80-0.91 (m, 12H), 1.18-1.35 (m, |
| | 64H), 1.49-1.73 (m, 12H), 2.10-2.3.05 (m, 22H), 3.79-3.81 (m, |
| | 2H), 4.84-4.87 (m, 2H); LCMS (ESI) m/z 934 [M + H]⁺ |
| Compound 28 | ¹H NMR (400 MHz, CDCl₃) δ 0.86-0.89 (m, 12H), 1.20-1.32 (m, |
| | 40H), 1.36-1.45 (m, 14H), 1.56-1.63 (m, 12H), 2.30-2.56 (m, |
| | 20H), 2.76 (t, J = 7.3 Hz, 4H), 3.61 (t, J = 5.3 Hz, 2H), |
| | 4.04-4.08 (m, 8H); LCMS (ESI) m/z 1008 [M + H]⁺ |
| Compound 29 | ¹H NMR (400 MHz, CDCl₃) δ 0.80-0.90 (m, 12H), 1.20-1.35 (m, |
| | 40H), 1.45-1.55 (m, 14H), 1.56-1.75 (m, 14H), 2.25-3.10 (m, |
| | 24H), 3.80-3.90 (m, 2H), 4.03-4.08 (m, 8H); LCMS (ESI) m/z |
| | 1022 [M + H]⁺ |
| Compound 30 | ¹H NMR (400 MHz, CDCl₃) δ 0.87 (t, J = 6.8 Hz, 12H), 1.20- |
| | 1.32 (m, 64H), 1.48-1.57 (m, 8H), 1.66-1.70 (m, 4H), 2.37- |
| | 2.69 (m, 20H), 2.86-2.89 (m, 4H), 4.84-4.90 (m, 2H); LCMS |
| | (ESI) m/z 933 [M + H]⁺ |
| Compound 31 | ¹H NMR (400 MHz, CDCl₃) δ 0.87 (t, J = 6.4 Hz, 12H), 1.21- |
| | 1.35 (m, 64H), 1.48-1.66 (m, 12H), 2.26-2.47 (m, 20H), 2.79- |
| | 2.82 (m, 4H), 3.57 (s, 4H), 4.82-4.88 (m, 2H), 7.21-7.29 (m, |
| | 10H); LCMS (ESI) m/z 1114 [M + H]⁺ |
| Compound 32 | ¹H NMR (400 MHz, CDCl₃) δ 0.87 (t, J = 6.4 Hz. 12H), 1.20- |
| | 1.35 (m, 64H), 1.47-1.55 (m, 8H), 1.65-1.75 (m, 4H), 2.23 (s, |
| | 6H), 2.39-2.73 (m, 24H), 4.85-4.87 (m, 2H); LCMS (ESI) m/z |
| | 962 [M + H]⁺ |
| Compound 33 | ¹H NMR (400 MHz, CDCl₃) δ 0.80-0.90 (m, 12H), 1.20-1.50 (m, |
| | 72H), 1.51-1.70 (m, 12H) 2.07-2.25 (m, 8H), 2.28-2.61 (m, |
| | 32H), 5.62 (d, J = 6.8 Hz, 8H), 5.49-5.55 (m, 4H), 5.61-5.65 |
| | (m, 4H); LCMS (ESI) m/z 1322 [M + H]⁺ |
| Compound 34 | ¹H NMR (400 MHz, CDCl₃) δ 0.88 (t, J = 6.8 Hz, 12H), 1.20- |
| | 1.45 (m, 40H), 1.56-1.70 (m, 20H), 2.05-2.15 (m, 8H), 2.25- |
| | 2.55 (m, 32H), 2.75 (t, J = 7.6 Hz, 8H), 4.05 (t, J = 6.80 |
| | Hz, 8H), 4.62 (t, J = 8.0 Hz, 8H), 5.48-5.54 (m, 4H), 5.60- |
| | 5.64 (m, 4H); LCMS (ESI) m/z 1442 [M + H]⁺ |
| Compound 35 | ¹H NMR (400 MHz, MeOD) δ 0.80-0.90 (m, 12H), 1.29-1.41 (m, |
| | 32H), 1.61-1.68 (m, 4H), 1.93-1.96 (m, 8H), 2.10-2.15 (m, |
| | 8H), 2.39-2.56 (m, 32H), 2.73-2.76 (m, 8H), 4.10 (t, J = 6.0 |
| | Hz, 8H), 4.64 (d, J = 6.8 Hz, 8H), 5.49-5.55 (m, 4H), 5.61- |
| | 5.66 (m, 4H); LCMS (ESI) m/z 1329 [M + H]⁺ |
| Compound 36 | ¹H NMR (400 MHz, CDCl₃) δ 0.86-0.89 (m, 18H), 1.22-1.39 (m, |
| | 96H), 1.45-1.80 (m, 16H), 2.40-3.11 (m, 28H), 4.83-5.01 (m, |
| | 3H), 6.73 (brs, 1H); LCMS (ESI) m/z 1300 [M + H]⁺ |
| Compound 37 | ¹H NMR (400 MHz, MeOD) δ 0.91 (t, J = 8.0 Hz, 9H), 1.24- |
| | 1.60 (m, 31H), 1.65-1.75 (m, 2H), 1.95-2.09 (m, 12H), 2.25 |
| | (s, 3H), 2.38-2.70 (m, 18H), 3.68 (t, J = 6.0 Hz, 2H), 4.94- |
| | 5.00 (m, 1H), 5.32-5.40 (m, 6H); LCMS (ESI) m/z 716 [M + H]⁺ |
| Compound 38 | ¹H NMR (400 MHz, MeOD) δ 0.91 (t, J = 8.0 Hz, 9H), 1.20- |
| | 1.48 (m, 31H), 1.50-1.60 (m, 2H), 1.64-1.79 (m, 4H), 1.97- |
| | 2.10 (m, 12H), 2.25 (s, 3H), 2.32-2.75 (m, 24H), 4.94-5.01 |
| | (m, 1H), 5.28-5.42 (m, 6H); LCMS (ESI) m/z 757 [M + H]⁺ |
| Compound 39 | ¹H NMR (400 MHz, MeOD) δ 0.91 (t, J = 8.0 Hz, 18H), 1.26- |
| | 1.41 (m, 56H), 1.52-1.60 (m, 6H), 1.73-1.82 (m, 4H), 1.95- |
| | 2.10 (m, 24H), 2.37 (s, 6H), 2.54-2.90 (m, 24H), 4.96-5.02 |
| | (m, 2H), 5.31-5.41 (m, 12H); LCMS (ESI) m/z 1258 [M + H]⁺ |
| Compound 40 | ¹H NMR (400 MHz, CDCl₃) δ 0.95-1.05 (m, 8H), 1.10-1.35 (m, |
| | 12H), 1.60-1.85 (m, 28H), 2.20 (d, J = 7.2 Hz, 8H), 2.25-2.55 |
| | (m, 24H), 2.70-2.85 (m, 8H), 4.27 (s, 16H); LCMS (ESI) m/z |
| | 1162 [M + H]⁺ |
| Compound 41 | ¹H NMR (400 MHz, MeOD) δ 0.95-1.05 (m, 4H) 1.10-1.40 (m, |
| | 6H), 1.60-1.80 (m, 24H), 2.19 (d, J = 6.4 Hz, 4 H), 2.24 (s, |
| | 6H), 2.32-2.70 (m, 24H), 4.00-4.15 (m, 8H); LCMS (ESI) m/z |
| | 765 [M + H]⁺ |
| Compound 42 | ¹H NMR (400 MHz, CDCl₃) δ 0.85-0.95 (m, 12H), 1.25-1.40 (m, |
| | 16H), 1.60-1.65 (m, 10H), 2.20 (s, 6H), 2.30-2.60 (m, 32H), |
| | 2.65-2.70 (m, 4H), 4.12-4.20 (m, 12H); LCMS (ESI) m/z 941 |
| | [M + H]⁺ |
| Compound 43 | ¹H NMR (400 MHz, CDCl₃) δ 0.80-0.90 (m, 12H), 1.20-1.35 (m, |
| | 12H), 1.48-1.58 (m, 8H), 1.60-1.70 (m, 4H), 2.22 (s, 6H), |
| | 2.30-2.65 (m, 20H), 2.68-2.71 (m, 4H), 4.79-4.85 (m, 2H); |
| | LCMS (ESI) m/z 597 [M + H]⁺ |
| Compound 44 | ¹H NMR (400 MHz, DMSO-d₆) δ 0.88-0.90 (m, 24H), 1.10-1.40 |
| | (m, 30H), 1.45-1.60 (m, 20H), 2.10-2.40 (m, 32H), 2.60-2.70 |
| | (m, 8H), 4.06-4.07 (m, 12H), 4.69-4.72 (m, 2H); LCMS (ESI) |
| | m/z 1282 [M + H]⁺ |
| Compound 45 | ¹H NMR (400 MHz, CDCl₃) δ 0.80-0.90 (m, 24H), 1.25-1.40 (m, |
| | 16H), 1.58-1.63 (m, 4H), 1.69-1.76 (m, 20H), 2.23-2.78 (m, |
| | 40H), 4.08-4.11 (m, 16H); LCMS (ESI) m/z 1225 [M + H]⁺ |
| Compound 46 | ¹H NMR (400 MHz, CDCl₃) δ 0.88 (t, J = 6.4 Hz, 12H), 1.20- |
| | 1.38 (m, 64H), 1.40-1.60 (m, 16H), 1.70-1.80 (m, 4H), 2.20- |
| | 2.60 (m, 24H), 2.80-2.84 (m, 4H), 3.55-3.56 (m, 4H), 4.84- |
| | 4.87 (m, 2H); LCMS (ESI) m/z 1078 [M + H]⁺ |
| Compound 47 | ¹H NMR (400 MHz, CDCl₃) δ 0.88 (t, J = 6.6 Hz, 12H), 1.20- |
| | 1.50 (m, 56H), 1.50-1.70 (m, 12H), 2.22 (s, 6H), 2.27-2.49 |
| | (m, 22H), 4.06 (t, J = 6.6 Hz, 4H); LCMS (ESI) m/z 905 [M + H]⁺ |
| Compound 48 | ¹H NMR (400 MHz, CDCl₃) δ 0.88 (t, J = 6.8 Hz, 6H), 1.19- |
| | 1.47 (m, 40H), 1.64-1.70 (m, 4H), 2.10 (q, J = 6.8 Hz, 4H), |
| | 2.19-2.26 (m, 30H), 4.62 (d, J = 6.8 Hz, 4H), 5.48-5.58 (m, |
| | 2H), 5.60-5.69 (m, 2H); LCMS (ESI) m/z 789 [M + H]⁺ |
| Compound 49 | ¹H NMR (400 MHz, CDCl₃) δ 0.83-0.95 (m, 12H), 1.22-1.41 (m, |
| | 14H), 1.50-1.80 (m, 6H), 1.90-1.98 (m, 4H), 2.09-2.17 (m, |
| | 4H), 2.19 (s, 6H), 2.36-2.47 (m, 8H), 2.49-2.55 (m, 2H), 2.56- |
| | 2.60 (m, 2H), 2.65-2.72 (m, 2H), 3.00-3.08 (m, 2H), 3.59-3.65 |
| | (m, 2H), 4.00 (m, 4H), 4.12 (t, J = 6.4 Hz, 4H); LCMS (ESI) |
| | m/z 728 [M + H]⁺ |

### Experimental Example 1. Properties of Ionizable Lipids

To prepare LNP, cholesterol purchased from Sigma Aldrich (USA) was used, and DMG-PEG2000 (1,2-Dimyristoyl-rac-glycero-3-methoxypolyethylene glycol-2000) purchased from Avanti Polar Lipids (USA) was used as a PEG lipid. In addition, DOPE (1,2-dioleoyl-sn-glycero-3-phosphoethanolamine) and DSPC (1,2-Distearoyl-sn-glycero-3-PC) purchased from Avanti Polar Lipids (USA) were used as helper lipids.

Selected ionizable lipids 2-7, 9-13, 27-30, 32, 33, 36-38, 40-43, and 46-49 were mixed with DOPE (1,2-dioleoyl-sn-glycero-3-phosphoethanolamine), cholesterol, and PEG-lipid to produce F1-type lipid nanoparticles (LNPs). F2-type LNPs were prepared by mixing selected ionizable lipids from 1-7, 9, 10, 12-26, and 28-49 were mixed with DOPE, cholesterol, and PEG-lipid. Table 3 lists the composition information of the F1-type and F2-type LNPs.

TABLE 3

| | Mixing ratio (mole %) |
|---|---|
| | (Ionizable lipid:Helper |
| LNP | lipid:Cholesterol:PEG-lipid) |
| F1-type LNP | 50:10:38.5:1.5 |
| F2-type LNP | 26.5:20:51.5:2 |

For LNP preparation, NanoAssemblr Ignite™ (Precision Nanosystems, Inc., Canada) was used, and the total flow rate (TFR) was 12 mL/min. Ethanol was removed using an Amicon Ultra Centrifugal Filter, MWCO 10 kDa (Millipore, USA), a buffer was exchanged, and then concentration was performed. During dilution, concentration, and exchange, the prepared LNPs were diluted with 1×DPBS to remove ethanol, and at the same time, the LNPs were concentrated.

### [Method for Analyzing Physicochemical Properties of LNP]

A particle size, a Z-average diameter, and a polydispersity index (PI) were analyzed using Zetasizer Pro (Malvern Instruments, United Kingdom). The “PI” is a ratio that describes the homogeneity of the particle size distribution of a system. PI closer to 0 means monodisperse, and closer to 1 means polydisperse.

[mRNA Quantification and Encapsulation Efficiency (EE %) Measurement Method]

The mRNA content and EE % were measured using the Ribogreen RNA assay kit (Invitrogen, USA). Briefly, the mRNA concentration was measured for the prepared mRNA/LNP in the Triton X-100 solution treated group and the untreated group, and the ratio of mRNA encapsulated in LNP among total mRNA was calculated as EE %.

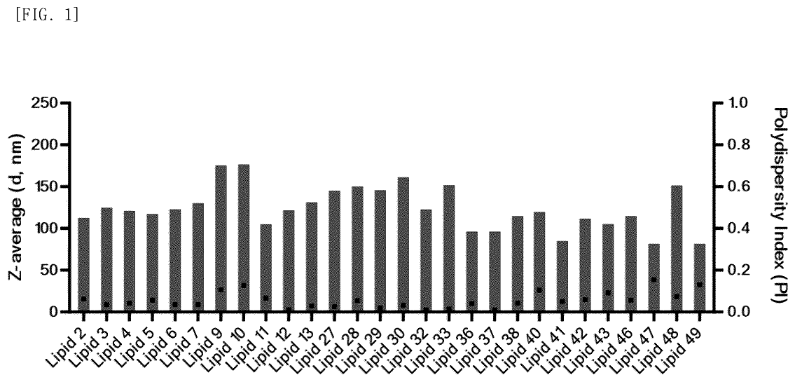

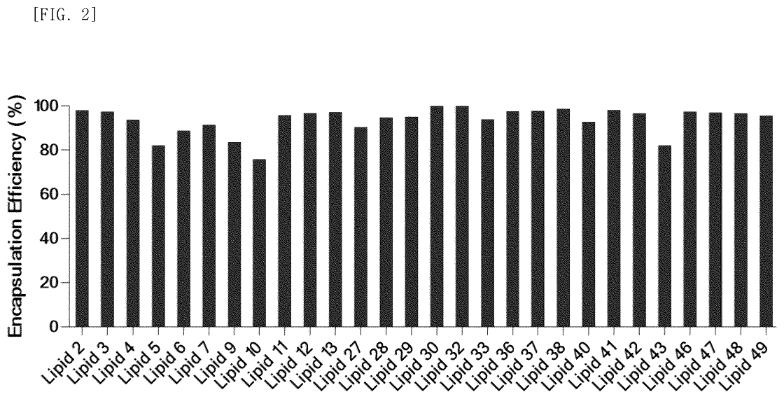

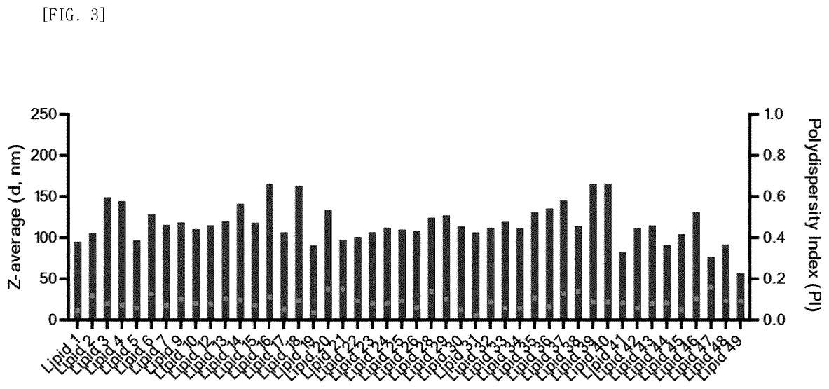

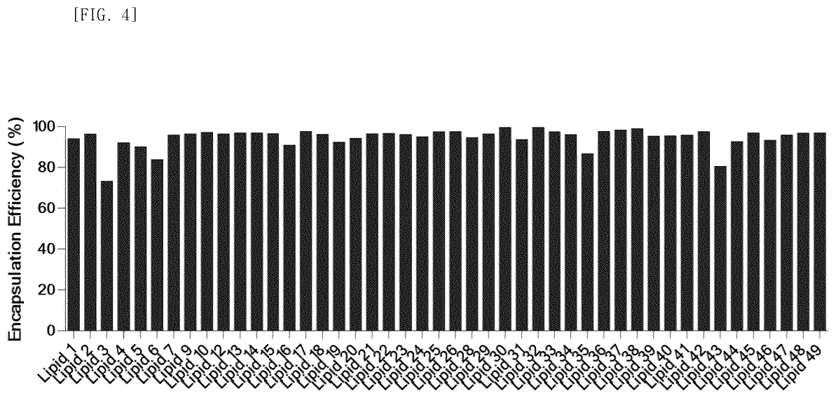

Table 4 below lists the Z-average diameter, PI, and EE (%) measured values for each LNP prepared, and these are shown in FIGS. 1 to 4, respectively.

TABLE 4

| | Z-average | | |
|---|---|---|---|
| | (nm) | PI | EE(%) |
| F1-type LNP | | | |
| LNP including ionizable lipid compound 2 (Lipid 2) | 112.7 | 0.0617 | 98.1 |
| LNP including ionizable lipid compound 3 (Lipid 3) | 124.8 | 0.0354 | 97.5 |
| LNP including ionizable lipid compound 4 (Lipid 4) | 121.1 | 0.0417 | 93.8 |
| LNP including ionizable lipid compound 5 (Lipid 5) | 117.4 | 0.0562 | 82.2 |
| LNP including ionizable lipid compound 6 (Lipid 6) | 123.0 | 0.0357 | 88.9 |
| LNP including ionizable lipid compound 7 (Lipid 7) | 130.3 | 0.0353 | 91.5 |
| LNP including ionizable lipid compound 9 (Lipid 9) | 175.6 | 0.1062 | 83.6 |
| LNP including ionizable lipid compound 10 (Lipid 10) | 176.8 | 0.1270 | 76.0 |
| LNP including ionizable lipid compound 11 (Lipid 11) | 105.0 | 0.0663 | 95.9 |
| LNP including ionizable lipid compound 12 (Lipid 12) | 121.8 | 0.0115 | 96.8 |
| LNP including ionizable lipid compound 13 (Lipid 13) | 131.3 | 0.0284 | 97.3 |
| LNP including ionizable lipid compound 27 (Lipid 27) | 145.4 | 0.0257 | 90.5 |
| LNP including ionizable lipid compound 28 (Lipid 28) | 150.4 | 0.0547 | 94.8 |
| LNP including ionizable lipid compound 29 (Lipid 29) | 146.0 | 0.0194 | 95.1 |
| LNP including ionizable lipid compound 30 (Lipid 30) | 161.4 | 0.0320 | 100.0 |
| LNP including ionizable lipid compound 32 (Lipid 32) | 122.9 | 0.0108 | 100.0 |
| LNP including ionizable lipid compound 33 (Lipid 33) | 152.0 | 0.0143 | 93.9 |
| LNP including ionizable lipid compound 36 (Lipid 36) | 96.3 | 0.0405 | 97.7 |
| LNP including ionizable lipid compound 37 (Lipid 37) | 96.3 | 0.0096 | 97.8 |
| LNP including ionizable lipid compound 38 (Lipid 38) | 114.8 | 0.0418 | 98.7 |
| LNP including ionizable lipid compound 40 (Lipid 40) | 119.8 | 0.1050 | 92.9 |
| LNP including ionizable lipid compound 41 (Lipid 41) | 85.2 | 0.0495 | 98.2 |
| LNP including ionizable lipid compound 42 (Lipid 42) | 111.7 | 0.0586 | 96.7 |
| LNP including ionizable lipid compound 43 (Lipid 43) | 105.4 | 0.0911 | 82.2 |
| LNP including ionizable lipid compound 46 (Lipid 46) | 114.9 | 0.0561 | 97.4 |
| LNP including ionizable lipid compound 47 (Lipid 47) | 81.8 | 0.1549 | 97.0 |
| LNP including ionizable lipid compound 48 (Lipid 48) | 151.6 | 0.0734 | 96.7 |
| LNP including ionizable lipid compound 49 (Lipid 49) | 81.7 | 0.1302 | 95.6 |
| F2-type LNP | | | |
| LNP including ionizable lipid compound 1 (Lipid 1) | 95.4 | 0.0451 | 94.3 |
| LNP including ionizable lipid compound 2 (Lipid 2) | 105.5 | 0.1188 | 96.5 |
| LNP including ionizable lipid compound 3 (Lipid 3) | 149.2 | 0.0782 | 73.5 |
| LNP including ionizable lipid compound 4 (Lipid 4) | 144.7 | 0.0713 | 62.1 |
| LNP including ionizable lipid compound 5 (Lipid 5) | 96.7 | 0.0563 | 90.3 |
| LNP including ionizable lipid compound 6 (Lipid 6) | 128.9 | 0.1276 | 84.0 |
| LNP including ionizable lipid compound 7 (Lipid 7) | 115.8 | 0.0696 | 96.2 |
| LNP including ionizable lipid compound 9 (Lipid 9) | 118.7 | 0.1004 | 96.7 |
| LNP including ionizable lipid compound 10 (Lipid 10) | 110.6 | 0.0805 | 97.4 |
| LNP including ionizable lipid compound 12 (Lipid 12) | 115.4 | 0.0765 | 96.6 |
| LNP including ionizable lipid compound 13 (Lipid 13) | 120.2 | 0.1021 | 97.1 |
| LNP including ionizable lipid compound 14 (Lipid 14) | 141.3 | 0.0969 | 97.1 |
| LNP including ionizable lipid compound 15 (Lipid 15) | 118.4 | 0.0711 | 96.8 |
| LNP including ionizable lipid compound 16 (Lipid 16) | 166.0 | 0.1116 | 91.1 |
| LNP including ionizable lipid compound 17 (Lipid 17) | 106.8 | 0.0514 | 97.9 |
| LNP including ionizable lipid compound 18 (Lipid 18) | 163.5 | 0.0944 | 96.5 |
| LNP including ionizable lipid compound 19 (Lipid 19) | 90.7 | 0.0348 | 92.6 |
| LNP including ionizable lipid compound 20 (Lipid 20) | 134.2 | 0.1509 | 94.4 |
| LNP including ionizable lipid compound 21 (Lipid 21) | 97.7 | 0.1515 | 96.7 |
| LNP including ionizable lipid compound 22 (Lipid 22) | 101.1 | 0.0920 | 96.9 |
| LNP including ionizable lipid compound 23 (Lipid 23) | 106.8 | 0.0779 | 96.4 |
| LNP including ionizable lipid compound 24 (Lipid 24) | 112.3 | 0.0805 | 95.2 |
| LNP including ionizable lipid compound 25 (Lipid 25) | 110.1 | 0.0929 | 97.7 |
| LNP including ionizable lipid compound 26 (Lipid 26) | 108.3 | 0.0611 | 97.8 |
| LNP including ionizable lipid compound 28 (Lipid 28) | 124.4 | 0.1367 | 94.8 |
| LNP including ionizable lipid compound 29 (Lipid 29) | 127.4 | 0.1008 | 96.6 |
| LNP including ionizable lipid compound 30 (Lipid 30) | 113.8 | 0.0515 | 100.0 |
| LNP including ionizable lipid compound 31 (Lipid 31) | 106.6 | 0.0228 | 93.9 |
| LNP including ionizable lipid compound 32 (Lipid 32) | 112.3 | 0.0861 | 100.0 |
| LNP including ionizable lipid compound 33 (Lipid 33) | 119.4 | 0.0576 | 97.7 |
| LNP including ionizable lipid compound 34 (Lipid 34) | 111.3 | 0.0543 | 96.3 |
| LNP including ionizable lipid compound 35 (Lipid 35) | 131.1 | 0.1068 | 87.0 |
| LNP including ionizable lipid compound 36 (Lipid 36) | 135.8 | 0.0646 | 97.9 |
| LNP including ionizable lipid compound 37 (Lipid 37) | 145.3 | 0.1280 | 98.5 |
| LNP including ionizable lipid compound 38 (Lipid 38) | 114.0 | 0.1385 | 99.2 |
| LNP including ionizable lipid compound 39 (Lipid 39) | 165.7 | 0.0872 | 95.5 |
| LNP including ionizable lipid compound 40 (Lipid 40) | 165.7 | 0.0872 | 95.7 |
| LNP including ionizable lipid compound 41 (Lipid 41) | 82.4 | 0.0835 | 96.1 |
| LNP including ionizable lipid compound 42 (Lipid 42) | 112.2 | 0.0575 | 97.8 |
| LNP including ionizable lipid compound 43 (Lipid 43) | 115.1 | 0.0791 | 80.7 |
| LNP including ionizable lipid compound 44 (Lipid 44) | 91.1 | 0.0830 | 92.9 |
| LNP including ionizable lipid compound 45 (Lipid 45) | 104.4 | 0.0501 | 97.2 |
| LNP including ionizable lipid compound 46 (Lipid 46) | 131.7 | 0.1014 | 93.6 |
| LNP including ionizable lipid compound 47 (Lipid 47) | 77.1 | 0.1585 | 96.1 |
| LNP including ionizable lipid compound 48 (Lipid 48) | 92.1 | 0.0925 | 97.0 |
| LNP including ionizable lipid compound 49 (Lipid 49) | 56.8 | 0.0898 | 97.1 |

As seen in Table 4, the LNPs including the ionizable lipid compounds herein were confirmed to have a Z-average diameter of less than 200 nm and a narrow particle size distribution with a PI of less than 0.2.

### Experimental Example 2. Nucleic Acid Delivery Efficacy of Novel Ionizable Lipid Compounds (In Vivo)

To evaluate in vivo transfection efficiency and distribution, the prepared material was administered by intravenous and intramuscular injections. PBS was used as a control material, and the F1-type LNPs or F2-type LNPs prepared in Experimental example 1 were used as an experimental group material.

### Experimental Example 2.1. Nucleic Acid Delivery Efficacy According to Intravenous Administration of Novel Ionizable Lipid Compounds

For intravenous administration group, lipid nanoparticles equivalent to 0.5 mg/kg mRNA were injected into Balb/c mice (male, 5 weeks old) by tail vein administration. At 6 hours after injection, 150 mg/kg of D-Luciferin (Perkin Elmer, USA) was administered intraperitoneally, and 15 minutes later, the luminescence intensity (Total flux) in each mouse liver tissue was quantitatively analyzed using the IVIS imaging system Luminar XR (Perkin Elmer, USA) equipment to measure the in vivo nucleic acid delivery efficacy of the novel ionizable lipid compound.

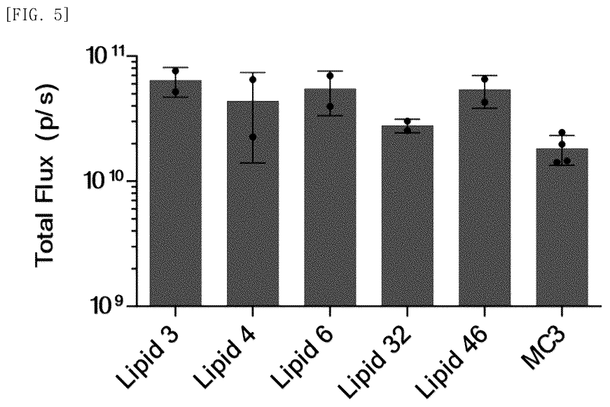

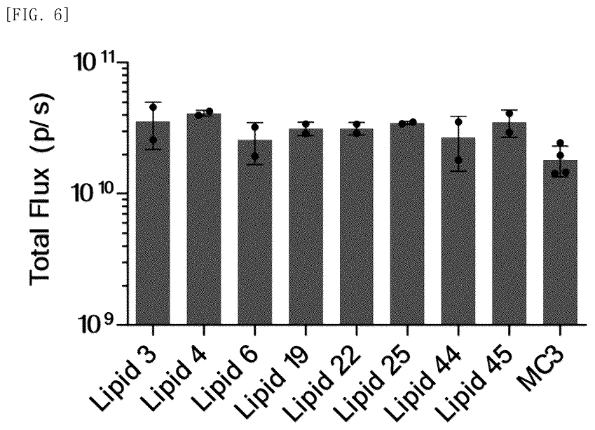

The quantified luminescence intensity for the experimental and control groups is summarized in Table 5. In addition, the relative luminescence intensity in the experimental and control groups is shown in FIGS. 5 and 6.

TABLE 5

| | Average |
|---|---|
| | Total Flux |
| F1-type LNP | |
| LNP including ionizable lipid compound 3 (Lipid 3) | 6.4 × 10¹⁰ |
| LNP including ionizable lipid compound 4 (Lipid 4) | 4.4 × 10¹⁰ |
| LNP including ionizable lipid compound 6 (Lipid 6) | 5.5 × 10¹⁰ |
| LNP including ionizable lipid compound 32 (Lipid 32) | 2.8 × 10¹⁰ |
| LNP including ionizable lipid compound 46 (Lipid 46) | 5.4 × 10¹⁰ |
| Control group (MC3 LNP) | 1.8 × 10¹⁰ |
| F2-type LNP | |
| LNP including ionizable lipid compound 3 (Lipid 3) | 3.6 × 10¹⁰ |
| LNP including ionizable lipid compound 4 (Lipid 4) | 4.1 × 10¹⁰ |
| LNP including ionizable lipid compound 6 (Lipid 6) | 2.6 × 10¹⁰ |
| LNP including ionizable lipid compound 19 (Lipid 19) | 3.2 × 10¹⁰ |
| LNP including ionizable lipid compound 22 (Lipid 22) | 3.2 × 10¹⁰ |
| LNP including ionizable lipid compound 25 (Lipid 25) | 3.5 × 10¹⁰ |
| LNP including ionizable lipid compound 44 (Lipid 44) | 2.7 × 10¹⁰ |
| LNP including ionizable lipid compound 45 (Lipid 45) | 3.5 × 10¹⁰ |
| Control group (MC3 LNP) | 1.8 × 10¹⁰ |

As confirmed in Table 5 above, compared to the control group, LNPs including the ionizable lipid compound herein were shown to have excellent in vivo mRNA delivery efficacy regardless of their composition.

### Experimental Example 2.2. Nucleic Acid Delivery Efficacy Following Intramuscular Administration of Novel Ionizable Lipid Compounds

For intramuscular administration group, lipid nanoparticles equivalent to 0.25 mg/kg mRNA were administered to the thigh muscles of Balb/c mice (male, 5 weeks old).

Control mice were treated with PBS in the same manner. At 6 hours after injection, 150 mg/kg of D-Luciferin (Perkin Elmer, USA) was administered intraperitoneally, and 15 minutes later, the luminescence intensity (Total flux) in each mouse thigh muscle tissue was quantitatively analyzed using the IVIS imaging system Luminar XR (Perkin Elmer, USA) equipment to measure the in vivo nucleic acid delivery efficacy of the novel ionizable lipid compound.

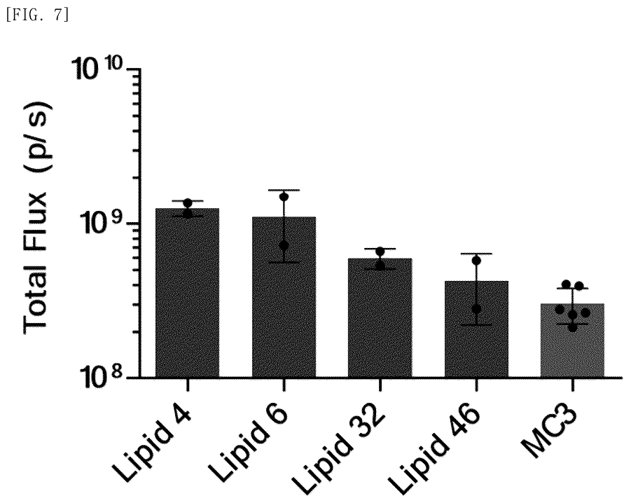

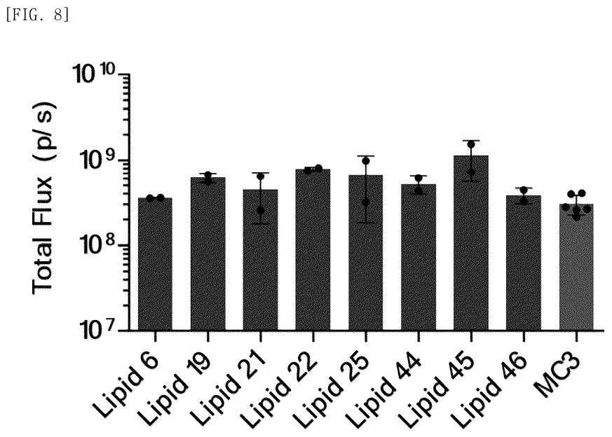

The quantified luminescence intensity in the experimental and control groups is summarized in Table 6. In addition, the relative luminescence intensity in the experimental and control groups is shown in FIGS. 7 and 8.

TABLE 6

| | Average |
|---|---|
| | Total Flux |
| F1-type LNP | |
| LNP including ionizable lipid compound 4 (Lipid 4) | 1.3 × 10⁹ |
| LNP including ionizable lipid compound 6 (Lipid 6) | 1.1 × 10⁹ |
| LNP including ionizable lipid compound 32 (Lipid 32) | 6.0 × 10⁸ |
| LNP including ionizable lipid compound 46 (Lipid 46) | 4.3 × 10⁸ |
| Control group (MC3 LNP) | 3.0 × 10⁸ |
| F2-type LNP | |
| LNP including ionizable lipid compound 6 (Lipid 6) | 3.6 × 10⁸ |
| LNP including ionizable lipid compound 19 (Lipid 19) | 6.2 × 10⁸ |
| LNP including ionizable lipid compound 21 (Lipid 21) | 4.5 × 10⁸ |
| LNP including ionizable lipid compound 22 (Lipid 22) | 7.9 × 10⁸ |
| LNP including ionizable lipid compound 25 (Lipid 25) | 6.6 × 10⁸ |
| LNP including ionizable lipid compound 44 (Lipid 44) | 5.2 × 10⁸ |
| LNP including ionizable lipid compound 45 (Lipid 45) | 1.1 × 10⁹ |
| LNP including ionizable lipid compound 46 (Lipid 46) | 3.8 × 10⁸ |
| Control group (MC3 LNP) | 3.0 × 10⁸ |

As confirmed in Table 6 above, compared to the control group, LNPs including the ionizable lipid compound herein were found to have significantly increased in vivo mRNA delivery efficacy regardless of their composition.

1. A compound represented by formula (I) below or a salt thereof:
(terminal area 1)ⱼ-connection1-headₖ-connection 2-(terminal area 2)ₘ (I),
wherein the terminal areas 1 and 2 are each independently represented by formula (II) or formula (III) below, 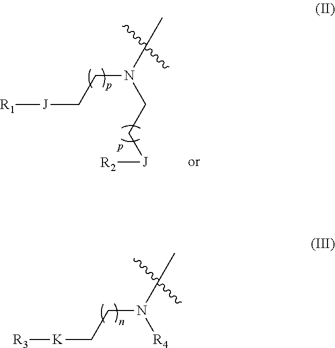
J and K are each independently —C(═O)—O— or —O—C(═O)—,
R₁, R₂ and R₃ are each independently linear C₁₋₂₅ alkyl or branched C₃₋₃₅ alkyl, R₁ and R₂ are the same as or different from each other, the linear C₁₋₂₅ alkyl or branched C₃₋₃₅ alkyl is substituted or unsubstituted with at least one selected from the group consisting of C₁₋₆ alkyl, Rx′-S—S—, Rx′-CH═CH—, Rx′-C(═O)—O—, Rx′-O—C(═O)—, —S—S—, —CH═CH—, —C(═O)—O—, —O—C(═O)—, 3- to 6-membered saturated or unsaturated carbocyclyl substituted or unsubstituted with Rx″, 3- to 6-membered saturated or unsaturated carbocycloalkyl substituted or unsubstituted with Rx″, 3- to 6-membered saturated or unsaturated heterocyclyl substituted or unsubstituted with Rx″, and 3- to 6-membered saturated or unsaturated heterocycloalkyl substituted or unsubstituted with Rx″,
R₄ is hydrogen, linear C₁₋₂₅ alkyl, branched C₃₋₃₀ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and R₄ is substituted or unsubstituted with one or more groups selected from the group consisting of Rx″, Rx′-CH═CH—, —CH═CH—, Rx′-C(═O)—O—, —C(═O)—O—, Rx′-O—C(═O)— and —O—C(═O)—,
Rx′ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3 to 6-membered saturated or unsaturated carbocycloalkyl, 3- to 6-membered saturated or unsaturated heterocyclyl, or 3- to 6-membered saturated or unsaturated heterocycloalkyl, and is substituted or unsubstituted with one or more Rx″,
Rx″ is a hydroxy group, linear C₁₋₁₅ alkyl, branched C₃₋₁₅ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl,
the wavy line indicates a binding position,
the terminal areas 1 and 2 are the same as or different from each other,
the connections 1 and 2 are each independently a single bond, linear C₁₋₆ alkyl, branched C₃₋₆ alkyl or C₁₋₆ alkylene, substituted or unsubstituted with a hydroxy group, —NR_{A}R_{B} or amine group, R_{A} and R_{B} are each independently linear C₁₋₆ alkyl,
the connections 1 and 2 are the same as or different from each other,
the head is a 6-membered heterocyclic amine or an aliphatic amine,
j and m are each independently an integer of 0 to 2, and cannot be an integer of 0 at the same time,
p and n are each independently integers from 1 to 20,
k is an integer of 0 or 1,
the heterocyclic amine is a cyclic amine group including one or more heteroatoms selected from N, S or O, and including at least one nitrogen atom,
the carbocyclyl is a cyclic group consisting of carbon and hydrogen atoms,
the heterocyclyl is a cyclic group including one or more heteroatoms selected from N, S or O, the carbocycloalkyl is a carbocyclyl-linear C₁₋₆ alkyl group or a carbocyclyl-branched C₃₋₆ alkyl group, and
the heterocycloalkyl is a heterocyclyl-linear C₁₋₆ alkyl group or a heterocyclyl-branched C₃₋₆ alkyl group.

2. The compound or salt thereof of claim 1, wherein
k is an integer of 1, and
the 6-membered heterocyclic amine of the head is piperidine, pyridine, pyrimidine, or piperazine.

3. The compound or salt thereof of claim 1, wherein
k is an integer of 1, and
the head is an aliphatic amine.

4. The compound or salt thereof of claim 1, wherein
k is 1,
the head is piperazine or piperidine,
the connections 1 and 2 are each independently linear C₁₋₆ alkyl substituted or unsubstituted with a hydroxy group, —NR_{A}R_{B} or an amine group, a branched C₃₋₆ alkyl substituted or unsubstituted with a hydroxy group, —NR_{A}R_{B} or an amine group, or C₁₋₆ alkylene substituted or unsubstituted with a hydroxy group, —NR_{A}R_{B} or an amine group, and R_{A} and R_{B} are each independently linear C₁₋₃ alkyl.

5. The compound or salt thereof of claim 1, wherein
both terminal areas 1 and 2 are represented by formula (II), or both terminal areas 1 and 2 are represented by formula (III).

6. The compound or salt thereof of claim 1, wherein
either of the terminal areas 1 and 2 is represented by formula (II), and the other terminal area is represented by formula (III).

7. The compound or salt thereof of claim 1, wherein R₁, R₂ and R₃ are each independently linear C₁₋₂₅ alkyl or branched C₃₋₃₂ alkyl substituted or unsubstituted with one or more selected from the group consisting of linear C₁₋₁₈ alkyl, 6-membered saturated or unsaturated carbocyclyl, 6-membered saturated or unsaturated carbocyclylalkyl, Rx′-CH═CH—, —CH═CH—, Rx′-C(═O)—O—, —C(═O)—O—, Rx′-O—C(═O)— and —O—C(═O),
Rx′ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and Rx′ is substituted or unsubstituted with one or more Rx″,
and Rx″ is a hydroxy group, linear C₁₋₁₅ alkyl, branched C₃₋₁₅ alkyl, 3- to 6-membered saturated or unsaturated carbocyclyl, 3- to 6-membered saturated or unsaturated carbocycloalkyl, 3- to 6-membered one-membered saturated or unsaturated heterocyclyl, or 3- to 6-membered saturated or unsaturated heterocycloalkyl.

8. The compound or salt thereof of claim 1, wherein
R₁, R₂ and R₃ are each independently linear C₁₋₂₅ alkyl or branched C₃₋₃₂ alkyl substituted or unsubstituted with one or more selected from the group consisting of linear C₂₋₁₈ alkyl, 6-membered saturated or unsaturated carbocyclyl, 6-membered saturated or unsaturated carbocyclylalkyl, —Rx′-CH═CH—, —CH═CH—, Rx′-C(═O)—O—, —C(═O)—O—, Rx′-O—C(═O)— and —O—C(═O)—,
Rx′ is hydrogen, linear C₁₋₂₅ alkyl, branched C₃₋₂₅ alkyl, linear C₅₋₃₀ alkenyl, branched C₅₋₃₀ alkenyl, 6-membered saturated or unsaturated carbocyclyl, 6-membered saturated or unsaturated carbocyclyl, 6-membered saturated or unsaturated carbocycloalkyl, 6-membered saturated or unsaturated heterocyclyl, or 6-membered saturated or unsaturated heterocycloalkyl, and Rx′ is substituted or unsubstituted with one or more Rx″, and
Rx″ is a hydroxyl group, linear C₁₋₁₅ alkyl, 6-membered saturated or unsaturated carbocyclyl, 6-membered saturated or unsaturated carbocyclylalkyl, 6-membered saturated or unsaturated heterocyclyl, or 6-membered saturated or unsaturated heterocyclylalkyl.

9. The compound or salt thereof of claim 1, wherein p and/or n are integers from 1 to 10.

10. The compound or salt thereof of claim 1, wherein R₄ is hydrogen, linear C₁₋₂₅ alkyl, branched C₃₋₃₀ alkyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and R₄ is substituted or unsubstituted with one or more groups selected from the group consisting of Rx″, Rx′-CH═CH—, —CH═CH—, Rx′-C(═O)—O—, —C(═O)—O—, Rx′-O—C(═O)— and —O—C(═O)—,
Rx′ is linear C₁₋₂₅ alkyl, branched C₃₋₃₀ alkyl, linear C₅₋₃₀ alkenyl, or branched C₅₋₃₀ alkenyl, and is substituted or unsubstituted with one or more Rx″, and
Rx″ is a hydroxy group, linear C₁₋₁₅ alkyl, branched C₃₋₁₅ alkyl, 3- to 6-membered saturated or unsaturated carbocyclyl, or 3- to 6-membered saturated or unsaturated carbocycloalkyl.

11. The compound or salt thereof of claim 1, wherein
one of the connections 1 and 2 is linear C₁₋₃ alkyl substituted or unsubstituted with a hydroxy group, —NR_{A}R_{B}— or an amine group, branched C₃₋₆ alkyl, substituted or unsubstituted with a hydroxy group, NR_{A}R_{B}— or amine group, or linear C₁₋₃ alkylene substituted or unsubstituted with a hydroxyl group, NR_{A}R_{B}— or an amine group.

12. The compound or salt thereof of claim 1, wherein
the compound or salt thereof is represented by formula (V): 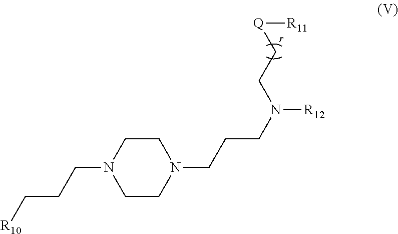
wherein r is an integer from 1 to 10,
Q is —C(═O)—O— or —O—C(═O)—,
R₁₀ is linear C₁₋₂₅ alkyl, or branched C₃₋₃₅ alkyl, or is a group represented by formula (VI), the linear C₁₋₂₅ alkyl, or branched C₃₋₃₅ alkyl is substituted or unsubstituted with one or more groups selected from the group consisting of a hydroxy group, linear C₅₋₃₀ alkenyl, branched C₅₋₃₀ alkenyl, and —NR_{C}R_{D} and amine groups, R_{C} and R_{D} are each independently linear C₁₋₆ alkyl,
R₁₂ is hydrogen, linear C₁₋₂₅ alkyl, linear C₁₋₂₅ alkyl substituted with a hydroxy group, linear C₁₋₂₅ alkyl substituted with 6-membered saturated or unsaturated carbocyclyl, 6-membered saturated or unsaturated carbocycloalkyl, or a group represented by formula (VI), 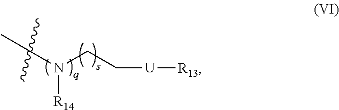
wherein the wavy line indicates a binding position,
U is —C(═O)—O— or —O—C(═O)—,
q is an integer of 0 or 1,
s is an integer from 1 to 10,
R₁₁, R₁₃ and R₁₄ are each independently hydrogen, linear C₁₋₂₅ alkyl, branched C₃₋₃₅ alkyl or 6-membered saturated or unsaturated carbocycloalkyl, the linear C₁₋₂₅ alkyl, branched C₃₋₃₅ alkyl or 6-membered saturated or unsaturated carbocycloalkyl is substituted or unsubstituted with at least one selected from the group consisting of a hydroxy group, C₁₋₆ alkyl, linear C₅₋₃₀ alkenyl, branched C₅₋₃₀ alkenyl, Rₐ—S—S—, —S—S—, Rₐ—CH═CH—, —CH═CH—, RₐC(═O)—O—, —C(═O)—O—, Rₐ—O—C(═O)—, —O—C(═O)—, 3- to 6-membered saturated or unsaturated carbocyclyl substituted or unsubstituted with R_{b}, 3- to 6-membered saturated or unsaturated carbocycloalkyl substituted or unsubstituted with R_{b}, 3- to 6-membered saturated or unsaturated heterocyclyl substituted or unsubstituted with R_{b}, and 3- to 6-membered saturated or unsaturated heterocycloalkyl substituted or unsubstituted with R_{b},
Rₐ is hydrogen, linear C₁₋₂₅ alkyl, branched C₃₋₂₅ alkyl, linear C₅₋₃₀ alkenyl, branched C₅₋₃₀ alkenyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl, and Rₐ is substituted or unsubstituted with R_{b},
R_{b} is linear C₁₋₁₅ alkyl, linear C₁₋₁₅ alkyl substituted with a hydroxy group, branched C₃₋₁₅ alkyl, 3- to 6-membered saturated or unsaturated carbocyclyl, 3- to 6-membered saturated or unsaturated carbocyclyl, 3- to 6-membered saturated or unsaturated carbocycloalkyl, 3- to 6-membered saturated or unsaturated heterocyclyl, or 3- to 6-membered saturated or unsaturated heterocycloalkyl,
the carbocyclyl is a cyclic group consisting of carbon and hydrogen atoms,
the heterocyclyl is a cyclic group including one or more heteroatoms selected from N, S or O,
the carbocycloalkyl is a carbocyclyl-linear C₁₋₆ alkyl group or a carbocyclyl-branched C₃₋₆ alkyl group, and
the heterocycloalkyl is a heterocyclyl-linear C₁₋₆ alkyl group or a heterocyclyl-branched C₃₋₆ alkyl group.

13. The compound or salt thereof of claim 12, wherein
R₁₀ is linear C₁₋₂₅ alkyl or branched C₃₋₃₅ alkyl, and is substituted or unsubstituted with a hydroxy group, linear C₅₋₃₀ alkenyl, branched C₅₋₃₀ alkenyl, —NR_{C}R_{D} or amine group, or a group represented by formula (VI), R_{C} and R_{D} are each independently linear C₁₋₃ alkyl, and
R₁₂ is hydrogen, linear C₁₋₆ alkyl, linear C₁₋₂₅ alkyl substituted with a hydroxy group, linear C₁₋₆ alkyl substituted with 6-membered saturated or unsaturated carbocyclyl, 6-membered saturated or unsaturated carbocycloalkyl, or a group represented by formula (VI).

14. The compound or salt thereof of claim 1, wherein the compound is selected from the group consisting of:
(1) Tetrakis(2-ethylhexyl) 3,3′,3″,3′″-((piperazine-1,4-diylbis(propane-3,1-diyl))bis(azanetriyl)) tetrapropionate;
(2) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrapentanoate;
(3) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetraheptanoate;
(4) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetranonanoate;
(5) Tetra(octan-3-yl) 3,3′,3″,3′″-((piperazine-1,4-diylbis(propane-3,1-diyl))bis(azanetriyl))tetrapropionate;
(6) Tetrakis(4-(2-cyclohexylacetoxy)butyl) 3,3′,3″,3′″-((piperazine-1,4-diylbis(propane-3,1-diyl))bis(azanetriyl))tetrapropionate;
(7) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetraundecanoate;
(8) Tetrakis(4-(propionyloxy)butyl) 3,3′,3″,3′″-((piperazine-1,4-diylbis(propane-3,1-diyl))bis(azanetriyl)) tetrapropionate;
(9) ((3,3′,3″,3′″-((Piperazine-1,4-diylbis(propane-3,1-diyl))bis(azanetriyl))tetrakis(propanoyl))tetrakis(oxy)) tetrakis(ethane-2,1-diyl) tetraheptanoate;
(10) ((3,3′,3″,3′″-((Piperazine-1,4-diylbis(propane-3,1-diyl))bis(azanetriyl))tetrakis(propanoyl))tetrakis(oxy)) tetrakis(propane-3,1-diyl) tetrahexanoate;
(11) ((3,3′,3″,3′″-((Piperazine-1,4-diylbis(propane-3,1-diyl))bis(azanetriyl))tetrakis(propanoyl))tetrakis(oxy)) tetrakis(pentane-5,1-diyl) tetrabutyrate;
(12) Tetrakis(2-ethylhexyl) 4,4′,4″,4′″-((3,3′,3″,3′″-((piperazine-1,4-diylbis(propane-3,1-diyl))bis(azanetriyl)) tetrakis(propanoyl))tetrakis(oxy))tetrabutyrate;
(13) Tetrakis(2-ethylhexyl) 6,6′,6″,6′″-((3,3′,3″,3′″-((piperazine-1,4-diylbis(propane-3,1-diyl))bis(azanetriyl)) tetrakis(propanoyl))tetrakis(oxy))tetrahexanoate;
(14) ((3,3′,3″,3′″-((Piperazine-1,4-diylbis(propane-3,1-diyl))bis(azanetriyl))tetrakis(propanoyl))tetrakis(oxy)) tetrakis(hexane-6,1-diyl) tetrakis(2-hexyldecanoate);
(15) ((3,3′,3″,3′″-((Piperazine-1,4-diylbis(propane-3,1-diyl))bis(azanetriyl))tetrakis(propanoyl))tetrakis(oxy)) tetrakis(octane-8,1-diyl) tetrakis(2-hexyldecanoate);
(16) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octakis(decanoate);
(17) Tetrakis(2-ethylhexyl) 8,8′,8″,8′″-((3,3′,3″,3′″-((piperazine-1,4-diylbis(propane-3,1-diyl))bis(azanetriyl)) tetrakis(propanoyl))tetrakis(oxy))tetraoctanoate;
(18) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrakis(2-hexyldecanoate);
(19) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octahexanoate;
(20) (((3,3′,3″,3′″-((Piperazine-1,4-diylbis(propane-3,1-diyl))bis(azanetriyl))tetrakis(propanoyl))tetrakis(oxy)) tetrakis(methylene))tetrakis(propane-2,1,3-triyl) octaoctanoate;
(21) Tetrakis(2-propylhexyl) 4,4′,4″,4′″-((3,3′,3″,3′″-((piperazine-1,4-diylbis(propane-3,1-diyl))bis (azanetriyl))tetrakis(propanoyl))tetrakis(oxy))tetrabutyrate;
(22) Tetrakis(2-ethylpentyl) 4,4′,4″,4′″-((3,3′,3″,3′″-((piperazine-1,4-diylbis(propane-3,1-diyl))bis(azanetriyl)) tetrakis(propanoyl))tetrakis(oxy))tetrabutyrate;
(23) Tetrakis(2-propylhexyl) 6,6′,6″,6′″-((3,3′,3″,3′″-((piperazine-1,4-diylbis(propane-3,1-diyl))bis (azanetriyl))tetrakis(propanoyl))tetrakis(oxy))tetrahexanoate;
(24) Tetrakis(2-propylhexyl) 8,8′,8″,8′″-((3,3′,3″,3′″-((piperazine-1,4-diylbis(propane-3,1-diyl))bis (azanetriyl))tetrakis(propanoyl))tetrakis(oxy))tetraoctanoate;
(25) Tetrakis(2-ethylpentyl) 6,6′,6″,6′″-((3,3′,3″,3′″-((piperazine-1,4-diylbis(propane-3,1-diyl))bis(azanetriyl)) tetrakis(propanoyl))tetrakis(oxy))tetrahexanoate;
(26) Tetrakis(2-ethylpentyl) 8,8′,8″,8′″-((3,3′,3″,3′″-((piperazine-1,4-diylbis(propane-3,1-diyl))bis(azanetriyl)) tetrakis(propanoyl))tetrakis(oxy))tetraoctanoate;
(27) Di(henicosan-10-yl) 3,3′-((3-(4-(3-hydroxypropyl)piperazin-1-yl)propyl)azanediyl)dipropionate;
(28) ((3,3′-((3-(4-(2-Hydroxyethyl)piperazin-1-yl)propyl)azanediyl)bis(propanoyl))bis(oxy))bis(hexane-6,1-diyl) bis(2-hexyldecanoate);
(29) ((3,3′-((3-(4-(3-Hydroxypropyl)piperazin-1-yl)propyl)azanediyl)bis(propanoyl))bis(oxy))bis(hexane-6,1-diyl) bis(2-hexyldecanoate);
(30) Di(henicosan-10-yl) 3,3′-((piperazine-1,4-diylbis(propane-3,1-diyl))bis(azanediyl))dipropionate;
(31) Di(henicosan-10-yl) 3,3′-((piperazine-1,4-diylbis(propane-3,1-diyl))bis(benzylazanediyl))dipropionate;
(32) Di(henicosan-10-yl) 3,3′-((piperazine-1,4-diylbis(propane-3,1-diyl))bis(methylazanediyl))dipropionate;
(33) Tetra((Z)-non-2-en-1-yl) 9,9′,9″,9′″-((piperazine-1,4-diylbis(propane-3,1-diyl))bis(azanetriyl)) tetranonanoate;
(34) Tetra((Z)-non-2-en-1-yl) 6,6′,6″,6′″-((3,3′,3″,3′″-((piperazine-1,4-diylbis(propane-3,1-diyl))bis (azanetriyl))tetrakis(propanoyl))tetrakis(oxy))tetrahexanoate;
(35) Tetra((Z)-non-2-en-1-yl) 4,4′,4″,4′″-((3,3′,3″,3′″-((piperazine-1,4-diylbis(propane-3,1-diyl))bis (azanetriyl))tetrakis(propanoyl))tetrakis(oxy))tetrabutyrate;
(36) Di(henicosan-10-yl) 3,3′-((3-(4-(3-((3-(henicosan-10-yloxy)-3-oxopropyl)amino)propyl)piperazin-1-yl) propyl)azanediyl)dipropionate;
(37) (6Z,16Z)-12-((Z)-Dec-4-en-1-yl)docosa-6,16-dien-11-yl 3-((3-(4-(2-hydroxyethyl)piperazin-1-yl) propyl)(methyl)amino)propanoate;
(38) (6Z,16Z)-12-((Z)-Dec-4-en-1-yl)docosa-6,16-dien-11-yl 3-((3-(4-(3-(dimethylamino)propyl)piperazin-1-yl)propyl)(methyl)amino)propanoate;
(39) Bis((6Z,16Z)-12-((Z)-dec-4-en-1-yl)docosa-6,16-dien-11-yl) 3,3′-((piperazine-1,4-diylbis(propane-3,1-diyl))bis(methylazanediyl))dipropionate;
(40) Tetrakis(2-(2-cyclohexylacetoxy)ethyl) 3,3′,3″,3′″-((piperazine-1,4-diylbis(propane-3,1-diyl))bis (azanetriyl))tetrapropionate;
(41) Bis(4-(2-cyclohexylacetoxy)butyl) 3,3′-((piperazine-1,4-diylbis(propane-3,1-diyl))bis(methylazanediyl)) dipropionate;
(42) (((3,3′-((Piperazine-1,4-diylbis(propane-3,1-diyl))bis(methylazanediyl))bis(propanoyl))bis(oxy))bis(methylene))bis(propane-2,1,3-triyl) tetrahexanoate;
(43) Di(octan-3-yl) 3,3′-((piperazine-1,4-diylbis(propane-3,1-diyl))bis(methylazanediyl))dipropionate;
(44) (((3,3′-((Piperazine-1,4-diylbis(propane-3,1-diyl))bis((3-(octan-3-yloxy)-3-oxopropyl)azanediyl))bis(propanoyl))bis(oxy))bis(methylene))bis(propane-2,1,3-triyl) tetrahexanoate;
(45) ((3,3′,3″,3′″-((Piperazine-1,4-diylbis(propane-3,1-diyl))bis(azanetriyl))tetrakis(propanoyl))tetrakis(oxy)) tetrakis(butane-4,1-diyl) tetrakis(3-ethylpentanoate);
(46) Di(henicosan-10-yl) 3,3′-((piperazine-1,4-diylbis(propane-3,1-diyl))bis((4-hydroxybutyl)azanediyl)) dipropionate;
(47) Bis(2-ethylhexyl) 3,3′-((3-(4-(3-(dimethylamino)propyl)piperazin-1-yl)propyl)azanediyl)dipropionate;
(48) ((3-(4-(3-(Dimethylamino)propyl)piperazin-1-yl)propyl)azanediyl)bis(hexane-6,1-diyl) bis(2-hexyldecanoate); and
(49) Di((Z)-non-2-en-1-yl) 9,9′-((3-(4-(3-(dimethylamino)propyl)piperazin-1-yl)propyl)azanediyl) dinonanoate.

15. Lipid nanoparticles comprising the compound of claim 1 or a salt thereof, a helper lipid, cholesterol, and a PEG lipid.

16. The Lipid nanoparticles of claim 15, wherein the helper lipid is a phospholipid.

17. A composition comprising:
an ionic drug; and
(i) the compound of claim 1 or a salt thereof, or (ii) lipid nanoparticles including the compound of claim 1 or a salt thereof, a helper lipid, cholesterol, and a PEG lipid.

18. The composition of claim 17, wherein the ionic drug is one or more selected from the group consisting of nucleic acid or nucleic acid-based drugs, peptides, protein drugs, protein-nucleic acid constructs, and ionic biopolymer-drug conjugates.

19. The composition of claim 18, wherein the nucleic acid is one or more selected from the group consisting of antisense oligonucleotide (ASO), short interfering RNA (siRNA), micro RNA (miRNA), self amplifying RNA (SAM), circular RNA, messenger RNA (mRNA), crRNA, tracrRNA, single guide RNA (sgRNA), transfer RNA (tRNA), asymmetric interference RNA (aiRNA), antagomir, ribozyme, dicer substrate RNA, short hairpin RNA (shRNA), plasmid DNA (pDNA), double stranded DNA (dsDNA), partial double stranded DNA, triple stranded DNA, partial triple stranded DNA, single stranded DNA (ssDNA), double stranded RNA (dsRNA), locked nucleic acid (LNA), peptide nucleic acid (PNA), miRNA analog or anti-miRNA.
